SEQUENCE LISTING (46 sequences)

SEQ ID NO: 1 - DNA, 1080 bp, synthetic construct
  misc_feature 1..1080: note = Description of sequence: Blood vessel epicardial substance (BVES) gene
  source 1..1080: mol_type = other DNA
atgaattatacagagtccagcccattgagagaatcaactgccataggttttacacctgagttagaaagtatcatacctgtgccttccaataagaccacttgtgaaaactggagagagatacatcatctggtttttcatgtagcaaatatttgttttgcagttgggttggttattccaactactcttcaccttcatatgatatttcttaggggaatgttaactctaggatgtaccctttatatcgtctgggccactctctaccgatgtgccttggatataatgatctggaactctgtgttcttgggtgtcaacattttgcatctgtcgtatcttttatacaagaagagaccggtaaagattgaaaaggaactcagtggcatgtaccggcgattgtttgaaccactccgtgtgcctccagatttgttcagaagactaactggacagttttgcatgatccaaaccttgaaaaagggccaaacttatgctgcagaggataaaacctcagttgatgaccgtctgagtattctcttgaagggaaaaatgaaggtctcctatcgaggacattttctgcataacatttacccctgtgcctttatagattctcctgaatttagatcaactcagatgcacaaaggtgaaaaattccaggtcaccattattgcagatgataactgcagatttttatgctggtcaagagaaagattaacatactttctggaatcagaacctttcttgtatgaaatctttaggtatcttattggaaaagacatcacaaataagctctactcattgaatgatcccaccttaaatgataaaaaagccaaaaagctggaacatcagctcagcctctgcacacagatctccatgttggaaatgaggaacagtatagccagctccagtgacagtgacgacggcttgcaccagtttcttcggggtacctccagcatgtcctctcttcatgtgtcatccccacaccagcgagcctctgccaagatgaaaccgatagaagaaggagcagaagatgatgatgacgtttttgaaccggcatctccaaatacattgaaagtccatcagctgcct

SEQ ID NO: 2 - protein, 360 aa, synthetic construct
  REGION 1..360: note = Description of sequence: BVES amino acid sequence
MNYTESSPLRESTAIGFTPELESIIPVPSNKTTCENWREIHHLVFHVANICFAVGLVIPTTLHLHMIFLRGMLTLGCTLYIVWATLYRCALDIMIWNSVFLGVNILHLSYLLYKKRPVKIEKELSGMYRRLFEPLRVPPDLFRRLTGQFCMIQTLKKGQTYAAEDKTSVDDRLSILLKGKMKVSYRGHFLHNIYPCAFIDSPEFRSTQMHKGEKFQVTIIADDNCRFLCWSRERLTYFLESEPFLYEIFRYLIGKDITNKLYSLNDPTLNDKKAKKLEHQLSLCTQISMLEMRNSIASSSDSDDGLHQFLRGTSSMSSLHVSSPHQRASAKMKPIEEGAEDDDDVFEPASPNTLKVHQLP

SEQ ID NO: 3 - DNA, 766 bp, synthetic construct
  misc_feature 1..766: note = Description of sequence: MHCK7
  source 1..766: mol_type = other DNA
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgactgtagatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagc

SEQ ID NO: 4 - DNA, 141 bp, synthetic construct
  misc_feature 1..141: note = Description of sequence: Inverted Terminal Repeat (ITR)
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 81 bp, synthetic construct
  misc_feature 1..81: note = Description of sequence: 3X hemagglutinin tag (3X HA) coding sequence
  source 1..81: mol_type = other DNA
tacccatacgatgttccagattacgcttacccatacgatgttccagattacgcttacccatacgatgttccagattacgct

SEQ ID NO: 6 - protein, 27 aa, synthetic construct
  REGION 1..27: note = Description of sequence: 3X hemagglutinin tag (3X HA)
YPYDVPDYAYPYDVPDYAYPYDVPDYA

SEQ ID NO: 7 - DNA, 211 bp, synthetic construct
  misc_feature 1..211: note = Description of sequence: Bovine growth hormone polyadenylation (bGH_polyA) signal
  source 1..211: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 8 - DNA, 6451 bp, synthetic construct
  misc_feature 1..6451: note = Description of sequence: BVES vector construct
  source 1..6451: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaatttccttcaaaattgtttttaaaggcctatttaatatcttaccctatctattccagcctgggcaacatagcaagatcctgtctctacaaaaaataaaaaataaaaaattagttgggcatggtgcctgtagtccagctacctgagaggccaaagcaggaggattgctggagcccaggagtttcatggttgtgctgctgcactccagccttgccaacagagtgacgctctgtcttaaagaaaaagaaaagaaaaacagaagcattactctctagttctcccattttcagtgaactaataaattattataccacacaatccataatttctcctatgccatgaacctttgttacctaaagttcttcctcattgtgtaggctaaacaaagaagctgggtaagaagagtgagtacgttttacagtttatatgtatcacttatataacgtaagtaaaattttgagcttatagctagttactaaaaacttaagaaattgcccagaaccgcataggttattgtggatttagagacgagtatacattgaccatgaaatatatgtttaaaaatagtctttctctgcatgtgtctttgtatgtctctctttctgtctttcacatatatacacacattatatgtatacatatatacatgtttttgtaactatatgttttgtgaattttgttttctcaaagttttgcctgaaaatttcaaaatattgaaaatgctttgatgtgtttgagattttataaaatattatccatcttatttaatcagctcaataacaaactgatcagtaagctttctgctgttttgccttttttttaatgcaggactcggaagatggaagaacttattttgtcaagatccatgccccttgggaggtattagttacctatgctgaagtcttgggaatcaaaatgcctattaaggagagtgatattccccgccctaagcacactcctataagctatgtgcttggacctgtaagactcccactgagtgtgaagtatccccatcctgaatattttactgcacaattcagcagacatcggcaggagctcttcctcatcgaagatcaggcaaccttctttccatcctcatcaagaaacagaattgtgtactatattctctcaagatgtccttttggcatagaagatgggaagaaaaggtttgggattgaaagactgctaaactctaacacttactcatctgcctatccactccatgatggccaatattggaagccatcagaacctcccaatcctaccaatgaaagatacacacttcaccagaattgggctcgattttcctatttctacaaggagcagcctttagacttgattaagaattattatggataactcgagcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgactgtagatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagctagcctcgagccaccatgaattatacagagtccagcccattgagagaatcaactgccataggttttacacctgagttagaaagtatcatacctgtgccttccaataagaccacttgtgaaaactggagagagatacatcatctggtttttcatgtagcaaatatttgttttgcagttgggttggttattccaactactcttcaccttcatatgatatttcttaggggaatgttaactctaggatgtaccctttatatcgtctgggccactctctaccgatgtgccttggatataatgatctggaactctgtgttcttgggtgtcaacattttgcatctgtcgtatcttttatacaagaagagaccggtaaagattgaaaaggaactcagtggcatgtaccggcgattgtttgaaccactccgtgtgcctccagatttgttcagaagactaactggacagttttgcatgatccaaaccttgaaaaagggccaaacttatgctgcagaggataaaacctcagttgatgaccgtctgagtattctcttgaagggaaaaatgaaggtctcctatcgaggacattttctgcataacatttacccctgtgcctttatagattctcctgaatttagatcaactcagatgcacaaaggtgaaaaattccaggtcaccattattgcagatgataactgcagatttttatgctggtcaagagaaagattaacatactttctggaatcagaacctttcttgtatgaaatctttaggtatcttattggaaaagacatcacaaataagctctactcattgaatgatcccaccttaaatgataaaaaagccaaaaagctggaacatcagctcagcctctgcacacagatctccatgttggaaatgaggaacagtatagccagctccagtgacagtgacgacggcttgcaccagtttcttcggggtacctccagcatgtcctctcttcatgtgtcatccccacaccagcgagcctctgccaagatgaaaccgatagaagaaggagcagaagatgatgatgacgtttttgaaccggcatctccaaatacattgaaagtccatcagctgcctggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggacgtacgaagaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 24 bp, synthetic construct
  misc_feature 1..24: note = Description of sequence: BVES KO Forward Primer
  source 1..24: mol_type = other DNA
acttgctttaaaaaacctcccaca

SEQ ID NO: 10 - DNA, 25 bp, synthetic construct
  misc_feature 1..25: note = Description of sequence: BVES KO Reverse Primer
  source 1..25: mol_type = other DNA
agtcactagcaagagatctgcaccc

SEQ ID NO: 11 - DNA, 25 bp, synthetic construct
  misc_feature 1..25: note = Description of sequence: BVES WT Forward Primer
  source 1..25: mol_type = other DNA
aagtgctgggattaaaggtgtgtgc

SEQ ID NO: 12 - DNA, 22 bp, synthetic construct
  misc_feature 1..22: note = Description of sequence: BVES WT Reverse Primer
  source 1..22: mol_type = other DNA
aaggacacatcacagcttcagg

SEQ ID NO: 13 - DNA, 24 bp, synthetic construct
  misc_feature 1..24: note = Description of sequence: Astrogin1 Forward Primer
  source 1..24: mol_type = other DNA
tagtaaggctgttggagctgatag

SEQ ID NO: 14 - DNA, 20 bp, synthetic construct
  misc_feature 1..20: note = Description of sequence: Astrogin1 Reverse Primer
  source 1..20: mol_type = other DNA
ctgcaccagtgtgcataagg

SEQ ID NO: 15 - DNA, 20 bp, synthetic construct
  misc_feature 1..20: note = Description of sequence: MuRF1 Forward Primer
  source 1..20: mol_type = other DNA
catcttccaggctgcgaatc

SEQ ID NO: 16 - DNA, 20 bp, synthetic construct
  misc_feature 1..20: note = Description of sequence: MuRF1 Reverse Primer
  source 1..20: mol_type = other DNA
actggagcactcctgcttgt

SEQ ID NO: 17 - DNA, 26 bp, synthetic construct
  misc_feature 1..26: note = Description of sequence: MUSA1 Forward Primer
  source 1..26: mol_type = other DNA
cttcagtctcgtggaatggtaatctt

SEQ ID NO: 18 - DNA, 21 bp, synthetic construct
  misc_feature 1..21: note = Description of sequence: MUSA1 Reverse Primer
  source 1..21: mol_type = other DNA
tgcagtactgaatcgccatac

SEQ ID NO: 19 - DNA, 21 bp, synthetic construct
  misc_feature 1..21: note = Description of sequence: Bnip3 Forward Primer
  source 1..21: mol_type = other DNA
tcctgggtagaactgcacttc

SEQ ID NO: 20 - DNA, 21 bp, synthetic construct
  misc_feature 1..21: note = Description of sequence: Bnip3 Reverse Primer
  source 1..21: mol_type = other DNA
gctgggcatccaacagtattt

SEQ ID NO: 21 - DNA, 19 bp, synthetic construct
  misc_feature 1..19: note = Description of sequence: p62 Forward Primer
  source 1..19: mol_type = other DNA
aggatggggacttggttgc

SEQ ID NO: 22 - DNA, 21 bp, synthetic construct
  misc_feature 1..21: note = Description of sequence: p62 Reverse Primer
  source 1..21: mol_type = other DNA
tcacagatcacattggggtgc

SEQ ID NO: 23 - DNA, 18 bp, synthetic construct
  misc_feature 1..18: note = Description of sequence: Beclin1 Forward Primer
  source 1..18: mol_type = other DNA
tggaagggtctaagacgt

SEQ ID NO: 24 - DNA, 19 bp, synthetic construct
  misc_feature 1..19: note = Description of sequence: Beclin1 Reverse Primer
  source 1..19: mol_type = other DNA
ggctgtggtaagtaatgga

SEQ ID NO: 25 - DNA, 22 bp, synthetic construct
  misc_feature 1..22: note = Description of sequence: Cts1 Forward Primer
  source 1..22: mol_type = other DNA
gtggactgttctcacgctcaag

SEQ ID NO: 26 - DNA, 20 bp, synthetic construct
  misc_feature 1..20: note = Description of sequence: Cts1 Reverse Primer
  source 1..20: mol_type = other DNA
tccgtccttcgcttcatagg

SEQ ID NO: 27 - DNA, 21 bp, synthetic construct
  misc_feature 1..21: note = Description of sequence: GAPDH Forward Primer
  source 1..21: mol_type = other DNA
aggtcggtgtgaacggatttg

SEQ ID NO: 28 - DNA, 23 bp, synthetic construct
  misc_feature 1..23: note = Description of sequence: GAPDH Reverse Primer
  source 1..23: mol_type = other DNA
tgtagaccatgtagttgaggtca

SEQ ID NO: 29 - DNA, 21 bp, synthetic construct
  misc_feature 1..21: note = Description of sequence: BVES Forward Primer
  source 1..21: mol_type = other DNA
ccgtgccttctaatgagacca

SEQ ID NO: 30 - DNA, 23 bp, synthetic construct
  misc_feature 1..23: note = Description of sequence: BVES Reverse Primer
  source 1..23: mol_type = other DNA
agaccacataaagggtacatcct

SEQ ID NO: 31 - DNA, 21 bp, synthetic construct
  misc_feature 1..21: note = Description of sequence: POPDC2 Forward Primer
  source 1..21: mol_type = other DNA
aggagctggaaaccggatgta

SEQ ID NO: 32 - DNA, 21 bp, synthetic construct
  misc_feature 1..21: note = Description of sequence: POPDC2 Reverse Primer
  source 1..21: mol_type = other DNA
gacgatgtctagtccacaagc

SEQ ID NO: 33 - DNA, 20 bp, synthetic construct
  misc_feature 1..20: note = Description of sequence: POPDC3 Forward Primer
  source 1..20: mol_type = other DNA
tgactgaacacccactctgc

SEQ ID NO: 34 - DNA, 22 bp, synthetic construct
  misc_feature 1..22: note = Description of sequence: POPDC3 Reverse Primer
  source 1..22: mol_type = other DNA
actgccacccataaaacctact

SEQ ID NO: 35 - DNA, 22 bp, synthetic construct
  misc_feature 1..22: note = Description of sequence: ATG7 Forward Primer
  source 1..22: mol_type = other DNA
tctgggaagccataaagtcagg

SEQ ID NO: 36 - DNA, 19 bp, synthetic construct
  misc_feature 1..19: note = Description of sequence: ATG7 Reverse Primer
  source 1..19: mol_type = other DNA
gcgaaggtcaggagcagaa

SEQ ID NO: 37 - DNA, 21 bp, synthetic construct
  misc_feature 1..21: note = Description of sequence: PARK2 Forward Primer
  source 1..21: mol_type = other DNA
tcttccagtgtaaccaccgtc

SEQ ID NO: 38 - DNA, 19 bp, synthetic construct
  misc_feature 1..19: note = Description of sequence: PARK2 Reverse Primer
  source 1..19: mol_type = other DNA
ggcagggagtagccaagtt

SEQ ID NO: 39 - DNA, 24 bp, synthetic construct
  misc_feature 1..24: note = Description of sequence: hADCY3-E4gRNA Forward Primer
  source 1..24: mol_type = other DNA
accgcgtacctgacgttctcgtga

SEQ ID NO: 40 - DNA, 24 bp, synthetic construct
  misc_feature 1..24: note = Description of sequence: hADCY3-E4gRNA Reverse Primer
  source 1..24: mol_type = other DNA
aaactcacgagaacgtcaggtacg

SEQ ID NO: 41 - DNA, 20 bp, synthetic construct
  misc_feature 1..20: note = Description of sequence: hADCY3 Forward Primer
  source 1..20: mol_type = other DNA
acctcatgctttccatcctg

SEQ ID NO: 42 - DNA, 20 bp, synthetic construct
  misc_feature 1..20: note = Description of sequence: hADCY3 Reverse Primer
  source 1..20: mol_type = other DNA
ccgctccaggtgatatctgt

SEQ ID NO: 43 - DNA, 24 bp, synthetic construct
  misc_feature 1..24: note = Description of sequence: hADCY6-Q205gRNA Forward Primer
  source 1..24: mol_type = other DNA
accgccgccaggactccatgtggg

SEQ ID NO: 44 - DNA, 24 bp, synthetic construct
  misc_feature 1..24: note = Description of sequence: hADCY6-Q205gRNA Reverse Primer
  source 1..24: mol_type = other DNA
aaaccccacatggagtcctggcgg

SEQ ID NO: 45 - DNA, 20 bp, synthetic construct
  misc_feature 1..20: note = Description of sequence: hADCY6 Forward Primer
  source 1..20: mol_type = other DNA
aggataccgaggtgacaacg

SEQ ID NO: 46 - DNA, 20 bp, synthetic construct
  misc_feature 1..20: note = Description of sequence: hADCY6 Reverse Primer
  source 1..20: mol_type = other DNA
gccaagatcaaatgcaaggt

(19) United States
(12) Patent Application Publication
(10) Pub. No.: US 2025/0340902 A1
(43) Pub. Date: Nov. 6, 2025

(54) GENE THERAPY FOR BVES-RELATED DISORDERS

(71) Applicant: OHIO STATE INNOVATION FOUNDATION, Columbus, OH (US)

(72) Inventors: Renzhi HAN, Camel, IN (US); Haiwen LI, Hangzhou City (CN)

(21) Appl. No.: 19/126,402

(22) PCT Filed: Nov. 1, 2023
(86) PCT No.: PCT/US2023/078326

§ 371 Date: May 1, 2025

Related U.S. Application Data
(60) Provisional application No. 63/421,383, filed on Nov. 1, 2022.

(51) Int. Cl. C12N 15/86; A61K 31/69; A61K 35/76; A61K 38/17; A61K 48/00; A61P 21/00; C07K 14/47

(52) U.S. Cl. CPC C12N 15/86; A61K 31/69; A61K 35/76; A61K 38/17; A61K 48/0058; A61P 21/00; C07K 14/47

(57) ABSTRACT

The present disclosure relates to compositions and methods for treating blood vessel epicardial substance (BVES) protein-related disorders.

### CROSS REFERENCE TO RELATED APPLICATIONS

This PCT application claims priority to, and the benefit of, U.S. Provisional Patent Application No. 63/421,383, filed Nov. 1, 2022, which is incorporated by reference herein in its entirety.

### STATEMENT REGARDING FEDERALLY SPONSORED RESEARCH

This invention was made with government support under grant number R01HL116546 awarded by the National Institutes of Health. The government has certain rights in the invention.

### REFERENCE TO SEQUENCE LISTING

The sequence listing submitted on Nov. 1, 2023, as an .XML file entitled “103361-338WO1_ST26.xml” created on Oct. 25, 2023, and having a file size of 67,991 bytes is hereby incorporated by reference pursuant to 37 C.F.R. § 1.52 (e) (5).

### FIELD

The present disclosure relates to compositions and methods for treating BVES-related disorders.

### BACKGROUND

Genetic mutations in BVES, which encode a cAMP binding protein known as blood vessel epicardial substance (also known as Popeye domain containing protein 1 or POPDC1), are responsible for limb-girdle muscular dystrophy type R25. Heart rhythm abnormalities including arrhythmia, atrioventricular (AV) block and sinus bradycardia have also been reported in limb-girdle muscular dystrophy type R25 (LGMDR25) patients. Currently, there are no treatments available for LGMDR25 patients. What is needed are new compositions and methods for treating BVES-related disorders.

### SUMMARY

The present disclosure provides methods of treating, decreasing, reducing, and/or preventing progression of BVES-related diseases or disorders.

In some aspects, disclosed herein is a method of treating muscular dystrophy in a subject in need thereof, comprising administering to the subject a therapeutically effective amount of an expression vector comprising a nucleic acid encoding a blood vessel epicardial substance (BVES) protein. In some embodiments, the nucleic acid is an RNA or a DNA.

In some embodiments, the nucleic acid encoding the BVES protein comprises a sequence at least 70% identical to SEQ ID NO: 1, or a fragment thereof. In some embodiments, the nucleic acid encoding the BVES protein comprises a sequence at least 80% identical to SEQ ID NO: 1, or a fragment thereof. In some embodiments, the nucleic acid encoding the BVES protein comprises a sequence at least 90% identical to SEQ ID NO: 1, or a fragment thereof. In some embodiments, the nucleic acid encoding the BVES protein comprises SEQ ID NO: 1, or a fragment thereof.

In some embodiments, the nucleic acid encoding the BVES protein is operatively linked to a muscle-specific promoter. In some embodiments, the muscle-specific promoter is MHCK7.

In some embodiments, the MHCK7 promoter comprises a sequence at least 70% identical to SEQ ID NO: 3, or a fragment thereof. In some embodiments, the MHCK7 promoter comprises a sequence at least 80% identical to SEQ ID NO: 3, or a fragment thereof. In some embodiments, the MHCK7 promoter comprises a sequence at least 90% identical to SEQ ID NO: 3, or a fragment thereof. In some embodiments, the MHCK7 promoter comprises SEQ ID NO: 3, or a fragment thereof.

In some embodiments, the nucleic acid encoding the BVES protein is operatively linked to an inverted terminal repeat (ITR) sequence. In some embodiments, the ITR sequence is at least 70% identical to SEQ ID NO: 4, or a fragment thereof. In some embodiments, the ITR sequence is at least 80% identical to SEQ ID NO: 4, or a fragment thereof. In some embodiments, the ITR sequence is at least 90% identical to SEQ ID NO: 4, or a fragment thereof. In some embodiments, the ITR sequence comprises SEQ ID NO: 4, or a fragment thereof.

In some embodiments, the expression vector comprises a sequence at least 70% identical SEQ ID NO: 8, or a fragment thereof. In some embodiments, the expression vector comprises a sequence at least 80% identical SEQ ID NO: 8, or a fragment thereof. In some embodiments, the expression vector comprises a sequence at least 90% identical SEQ ID NO: 8, or a fragment thereof. In some embodiments, the expression vector comprises SEQ ID NO: 8, or a fragment thereof.

In some embodiments, the expression vector is a viral vector. In some embodiments, the viral vector is an adeno-associated virus (AAV) vector. In some embodiments, the AAV vector is an AAV1 vector, an AAV2 vector, an AAV3 vector, an AAV4 vector, an AAV5 vector, an AAV6 vector, an AAV7 vector, an AAV8 vector, or an AAV9 vector. In some embodiments, the AAV vector is an AAV vector of serotype rh9, rh10, rh74, AAVmyo, or MyoAAV.

In some embodiments, the subject has limb-girdle muscular dystrophy type R25 (LGMDR25).

In some embodiments, the subject has a mutated BVES gene.

In some embodiments, the method of any preceding aspect further comprises administering to the subject a therapeutically effective amount of a proteasome inhibitor. In some embodiments, the proteasome inhibitor is bortezomib.

### BRIEF DESCRIPTION OF FIGURES

The accompanying figures, which are incorporated in and constitute a part of this specification, illustrate several aspects described below.

FIGS. 1A, 1B, 1C, 1D, 1E, 1F, 1G, 1H, and 1I show the BVES disruption compromises the body weight gain and muscle function in mice. FIG. 1A shows the immunofluorescence images of GA muscles in WT and BVES-KO mice stained with the antibody against BVES and DAPI. Scale bar: 100 μm. FIG. 1B shows the representative image of WT and BVES-KO littermates at 4 months of age. FIG. 1C shows the Body weight gain of male BVES-KO and age/sex-matched WT mice from two to five months of age. * P<0.05, ** P<0.01, **** P<0.0001. FIG. 1D shows the kaplan-Meier survival curve of WT and BVES-KO male mice. FIG. 1E shows the voluntary wheel running of BVES-KO and age-matched WT male mice. FIGS. 1F and 1G show the endurance capacity test performed by treadmill running showing running distance (FIG. 1F) and time to exhaustion (FIG. 1G) in BVES-KO and WT male mice. ** P<0.01 [unpaired Student t test]. FIG. 1H shows the number of drop-outs to test the capacity of recovery from muscle injury on the treadmill in BVES-KO and WT male mice. FIG. 1I shows the tetanic torque measurements of the posterior compartment muscles of BVES-KO and WT male mice in age-dependent manner. ns indicates no significant difference. ** P<0.01, *** P<0.001, **** P<0.0001

FIGS. 2A, 2B, 2C, 2D, 2E, 2F, 2G, 2H, 2I, 2J, 2K, 2L, and 2M show that BVES deficiency causes muscle atrophy and dystrophy in mice. FIG. 2A shows the photographs of mice after the removal of skin and muscle tissues dissected from BVES-KO and WT mice. FIGS. 2B and 2C show the net mass of QU (FIG. 2B) and GA (FIG. 2C) muscles in BVES-KO and WT mice at 4 and 8 months of age. FIGS. 2D and 2E show the relative mass of QU (FIG. 2D) and GA (FIG. 2E) muscles normalized to the body weight at 4 and 8 months age. FIG. 2F shows the H&E-stained images of GA muscle cross-sections from 8-month-old BVES-KO and WT mice. Scale bar: 100 μm. FIG. 2G shows the CSA distribution of GA muscle fibers in 8-month-old BVES-KO and WT mice. FIG. 2H shows the mean CSA of GA muscle fibers. * P<0.05, ** P<0.01. FIG. 2I shows the quantification of CNFs in the GA muscles. ** P<0.01, **** P<0.0001. FIG. 2J shows the CK measurements in 8-month-old BVES-KO and WT mice. * P<0.05. FIG. 2K shows the representative immunofluorescence images of BVES-KO and WT GA muscle sections stained with dystrophin (Dys, green) and one of the myosin heavy chain antibodies (MyHC-I, MyHC-IIa and MyHC-IIb, red). Scale Bar: 100 μm. FIG. 2L shows the mean CSA of MyHC-I, MyHC-IIa and MyHC-IIb fibers in GA muscles from BVES-KO and WT mice. *** P<0.001, FIG. 2M shows the percentage of MyHC-I, MyHC-IIa and MyHC-IIb fibers in GA muscles from BVES-KO and WT mice.

FIGS. 3A, 3B, 3C, 3D, 3E, 3F, 3G, 3H, 3I, and 3J show the muscle-specific expression of BVES ameliorates skeletal muscle pathology in BVES-KO mice. FIG. 3A shows the schematic map of AAV9-BVES constructs. FIG. 3B shows the immunofluorescence images of AAV9-BVES treated and contralateral control GA muscles in BVES-KO mice stained with the antibody against HA-tag and DAPI. Scale bar: 100 μm. FIG. 3C shows the western blot of BVES expression detected with anti-BVES and anti-HA antibodies in AAV9-BVES treated and contralateral control GA muscles. FIG. 3D shows the tetanic torque measurements of the posterior compartment muscles of AAV9-BVES treated and contralateral control GA muscles at 1 and 3 months after injection. * P<0.05, ** P<0.01, **** P<0.0001. FIGS. 3E and 3F show the relative mass of AAV9-BVES treated and contralateral control GA muscles (FIG. 3E) or untreated QU muscle (FIG. 3F) normalized by the body weight at 1 and 3 months after injection. FIG. 3G shows the H&E staining of AAV9-BVES treated or contralateral control GA muscle sections. Scale bar: 100 μm. FIG. 3H shows the CSA distribution of AAV9-BVES treated and contralateral control GA muscles from BVES-KO mice at 3 month after injection. FIG. 3I shows the mean CSA of AAV9-BVES treated and contralateral control GA muscles from BVES-KO mice at 3 months after injection. * P<0.05. FIG. 3J shows the quantification of CNFs in the AAV9-BVES treated or contralateral control GA muscles from BVES-KO mice. ** P<0.01.

FIGS. 4A, 4B, 4C, 4D, 4E, 4F, 4G, 4H, 4I, 4J, and 4K show the BVES interacts with ADCY9 and regulates cAMP/PKA signaling. FIG. 4A shows the diagram showing the IP-mass spectrometry approach to identify BVES-interacting proteins in mouse skeletal muscle. FIGS. 4B and 4C shows the gene ontology analysis of the cellular compartment (FIG. 4B) and biological process (FIG. 4C) categorization of co-immunoprecipitated proteins with BVES-HA. FIG. 4D shows the ADCY9 was co-immunoprecipitated with BVES-HA using the Flag antibody from the lysates of Cos-1 cells co-transfected with Flag-ADCY9 and BVES-HA. FIG. 4E shows the IBMX (500 μM)-induced CAMPr fluorescence changes in ADCY3/6 double mutant HEK293 cells with stable expression of CAMPr, transfected with the indicated plasmids. Cell number: 31, 15, 50, 30 for mCherry, BVES, ADCY9 and ADCY9+BVES, respectively, from three independent trials per condition. FIG. 4F shows the CAMP measurements in skeletal muscle lysates from BVES-KO and WT mice. ** P<0.01. FIGS. 4G and 4H show the western blot (FIG. 4G) and quantification (FIG. 4H) of p-PKA substrates in skeletal muscle from BVES-KO and WT mice. * P<0.05. FIGS. 4I, 4J, and 4K show the western blot (FIG. 4I) and quantification of phosphorylated LKB1(FIG. 4J) and phosphorylated AMPK (FIG. 4K) in skeletal muscle from BVES-KO and WT mice. * P<0.05. ** P<0.01.

FIGS. 5A, 5B, 5C, 5D, 5E, 5F, 5G, 5H, 5I, 5J, 5K, 5L, 5M, 5N, 5O, 5P, 5Q, and 5R show the BVES disruption enhances the UPS via FoxO. FIGS. 5A, 5B, 5C, 5D, and 5E show the western blot of FoxO signaling in total GA muscle extracts from 8-month-old WT and BVES-KO mice (FIG. 5A) and quantification of FoxO1 (FIG. 5B), FoxO3a (FIG. 5C), MuRF1 (FIG. 5D) and Atrogin-1 (FIG. 5E) normalized to GAPDH. * P<0.05, *** P<0.001. FIGS. 5F and 5G show the western blot (FIG. 5F) and quantification (FIG. 5G) of ubiquitination in total GA muscle extracts from 8-month-old WT and BVES-KO mice. * P<0.05. FIG. 5H shows the immunostaining of FoxO3a and Cav3 (labeling muscle fibers) of GA muscles from WT and BVES-KO. Scale bar: 50 μm. FIG. 5I shows the immunostaining of FoxO3a and Cav3 of GA muscles from AAV9-BVES treated and contralateral control GA muscles. Scale bar: 50 μm. FIG. 5J shows the body weight of BVES-KO mice at 2 months after vehicle or Bortezomib treatment. FIG. 5K shows the tetanic torque measurements of BVES-KO mice at 2 months after vehicle or Bortezomib treatment. FIGS. 5L and 5M show the treadmill running test showing time to exhaustion (FIG. 5L) and total running distance (FIG. 5M) of BVES-KO mice at 2 months after vehicle or Bortezomib treatment. FIG. 5N shows the normalized QU mass to body weight in BVES-KO mice with vehicle or bortezomib treatment. FIG. 5O shows the H&E staining of GA muscle sections from BVES-KO mice with vehicle or bortezomib treatment. FIG. 5P shows the CSA distribution of bortezomib treated and vehicle control GA muscles from BVES-KO mice at 2 months after treatment. FIG. 5Q shows the mean CSA of bortezomib treated and vehicle control GA muscles from BVES-KO mice at 2 months after treatment. ** P<0.01. FIG. 5R shows the quantification of CNFs in the bortezomib treated and vehicle control GA muscles. ** P<0.01.

FIGS. 6A, 6B, 6C, 6D, 6E, 6F, 6G, 6H, 6I, 6J, 6K, 6L, 6M, 6N and 6O show the BVES Disruption suppresses the autophagic execution. FIGS. 6A, 6B, 6C, and 6D show the western blot (FIG. 6A) of autophagic markers and quantification of LC3A (FIG. 6B), LC3B (FIG. 6C) and p62 (FIG. 6D) normalized to GAPDH in total GA muscle extracts from 8-months-old WT and BVES-KO mice. FIG. 6E shows the p62 and Cav3 immunostaining of GA muscles from 4-months-old WT and BVES-KO mice. Scale bar: 100 μm. FIGS. 6F and 6G show the western blot (FIG. 6F) and quantification (FIG. 6G) of VPS34 in total GA muscle extracts from 8-months-old WT and BVES-KO mice. FIGS. 6H and 6I show the live cell imaging (FIG. 6H) of RFP-LC3B and quantification (FIG. 6I) of LC3B puncta in FDB muscle fibers from 8-months-old WT and BVES-KO mice at 5 days after electroporation with pCMV-RFP-LC3B. * P<0.05. FIGS. 6J and 6K show the western blot (FIG. 6J) and quantification (FIG. 6K) of LC3B/A in GA muscles from BVES-KO and WT mice without or with colchicine treatment as indicated. Blots are representative of two independent experiments. FIGS. 6L and 6M show the live cell imaging (FIG. 6L) of mCherry-GFP-LC3B dual reporter in FDB muscle fibers from 8-month-old WT and BVES-KO mice at 5 days after electroporation and quantification (FIG. 6M) of red-only puncta normalized by total puncta. *** P<0.001. FIGS. 6N and 6O show the western blot (FIG. 6N) and quantification (FIG. 6O) of STX17 (S17) and VAMP7 (V7) normalized to GAPDH (G) in total GA muscle extracts from WT and BVES-KO mice. * P<0.05; *** P<0.001.

FIGS. 7A, 7B, 7C, 7D, 7E, 7F, 7G, and 7H show the development of BVES deficient mouse model and phenotype analysis. FIG. 7A shows the expression of Bves in different mouse tissues examined by RT-PCR. FIG. 7B shows the diagram showing the strategy to generate BVES-KO mice. FIG. 7C shows the expression of Bves in the skeletal muscle of WT, BVES-heterozygous (Het) and BVES-KO mice examined by quantitative RT-PCR. * P<0.05, **** P<0.001. FIGS. 7D and 7E show the western blot (FIG. 7D) and quantification (FIG. 7E) of BVES proteins in membrane extraction of WT, BVES-Het and BVES-KO skeletal muscles. Na/K-ATPase was used as a loading control for membrane fractions. The arrows labeled the specific BVES bands. FIG. 7F shows the expression of Bves, Popdc2 and Popdc3 in BVES-KO and WT skeletal muscles by quantitative RT-PCR. FIG. 7G shows the Body weight gain of BVES-KO and age-matched WT female mice from one to eight months of age. * P<0.05, ** P<0.01, *** P<0.001. FIG. 7H shows the tetanic torque measurements of the posterior compartment muscles of WT and BVES-Het mice at 5 and 12 months of age. *** P<0.001.

FIGS. 8A, 8B, 8C, 8D, 8E, and 8F show the muscle mass measurements in BVES-KO and WT male mice. FIGS. 8A, 8B, 8C show the net mass of tibial anterior (TA) (FIG. 8A), soleus (FIG. 8B) and heart (FIG. 8C) in BVES-KO and WT mice at 4 and 8 months of age. FIGS. 8D, 8E, and 8F show the relative mass of TA (FIG. 8D), soleus (FIG. 8E) and heart (FIG. 8F) normalized to the body weight in BVES-KO and WT mice at 4 and 8 months of age. ** P<0.01; *** P<0.001; **** P<0.0001; ns, not significant.

FIGS. 9A, 9B, 9C, 9D, 9E, 9F, 9G, 9H, 9I, and 9J show the muscle mass measurements in BVES-KO and WT female mice. FIGS. 9A, 9B, 9C, 9D, and 9E show the net mass of GA (FIG. 9A), QU (FIG. 9B), TA (FIG. 9C), soleus (FIG. 9D) and heart (FIG. 9E) in BVES-KO and WT female mice at 4 months of age. FIGS. 9F, 9G, 9H, 9I, and 9J show the relative mass of GA (FIG. 9F), QU (FIG. 9G), TA (FIG. 9H), soleus (FIG. 9I) and heart (FIG. 9J) normalized to the body weight in BVES-KO and WT female mice at 4 months of age. * P<0.05; ** P<0.01; *** P<0.001; **** P<0.0001; ns, not significant.

FIGS. 10A and 10B show the histopathology of 4-month-old BVES-KO and WT male mice. FIG. 10A show the representative H&E staining images of GA muscle cross-sections from BVES-KO and WT mice at 4 months of age. Scale bar: 100 μm. FIG. 10B show the CSA distribution of GA muscles from 4-month-old BVES-KO and WT mice.

FIG. 11 shows the generation of ADCY3/6 double KO HEK293 cell line with stable expression of cAMPr. The cAMPr stably transduced HEK293 cells were transfected with AncBE4max and the guide RNAs (gRNAs) targeting ADCY3 and ADCY6 to generate ADCY3_IVS4+1G>A and ADCY6_Q205X mutations. Sanger sequencing of single cell clone confirmed that this cell line carried the homozygous ADCY3 and ADCY6 mutations.

FIG. 12 shows the expression of ubiquitination E3 ligases in skeletal muscle. The transcript expression levels of Atrogin-1, Murf1 and Musa1 determined by quantitative RT-PCR. * P<0.05; ** P<0.01; **** P<0.0001.

FIG. 13 shows the improvement of muscle mass after bortezomib treatment. The normalized GA muscle mass to body weight in BVES-KO mice with vehicle or bortezomib treatment. ** P<0.01.

FIGS. 14A, 14B, 14C, 14D, 14E, 14F, and 14G show the unaffected protein biosynthesis in BVES-KO skeletal muscles. FIGS. 14A and 14B show the western blot (FIG. 14A) and quantification (FIG. 14B) of the puromycin-labeled peptides. ns, not significant. FIGS. 14C, 14D, 14E, 14F, and 14G show the western blot (FIG. 14C) and quantification of AKT signaling related with protein translation including p-AKT (T308) (FIG. 14D), p-AKT (S473) (FIG. 14E), p-4E-BP1 (T37/46) (FIG. 14F) and p-p70 S6K (T389) (FIG. 14G). * P<0.05; ** P<0.01; ns, not significant.

FIGS. 15A and 15B show the alternation of autophagy in BVES-KO skeletal muscles. FIG. 15A shows the immunostaining of p62 and Cav3 in GA muscles from 8-month-old WT and BVES-KO mice. Scale bar: 50 μm. FIG. 15B shows the mRNA levels of the autophagy associated genes p62, Bnip3. Atg7. Cts1, Becn1 and Park2 determined by quantitative RT-PCR. * P<0.05; ** P<0.01; *** P<0.001; **** P<0.0001.

FIGS. 16A, 16B, 16C, 16D, 16E, and 16F show the improvement of autophagy defect in BVES-KO skeletal muscle following AAV9-BVES gene transfer. FIGS. 16A, 16B, 16C, and 16D show the western blot (FIG. 16A) of autophagy markers in control and AAV9-BVES treated GA muscles from BVES-KO mice and quantification of LC3A (FIG. 16B), LC3B (FIG. 16C) and p62 (FIG. 16D) normalized by GAPDH. ** P<0.01; *** P<0.001. FIG. 16E shows the immunostaining of p62 and BVES-HA in control and AAV9-BVES treated GA muscles from BVES-KO mice. FIG. 16F shows the quantitative RT-PCR analysis of the genes related with autophagy in control and AAV9-BVES treated GA muscles from BVES-KO mice. * P<0.05; ** P<0.01.

FIG. 17 shows the expression of autophagy related proteins in BVES-KO skeletal muscles. Western blot analyses of autophagy related proteins in WT (n=5) and BVES-KO (n=5) GA muscles.

FIGS. 18A, 18B, 18C, and 18D show the human BVES was specifically and efficiently expressed in the striated muscles following i.p. injection of AAV9.BVES in neonatal BVES-KO mice. FIG. 18A shows the AAV9.BVES was widely expressed in various skeletal muscles and heart but not in the other tissues. Scale bar: 100 μm. FIGS. 18B and 18C show the western blot showed AAV9.BVES was highly expressed in GA muscle (FIG. 18B) and heart (FIG. 18C) in the male BVES-KO mice with neonatal injection of AAV9.BVES. Arrows indicate the specific bands of BVES. FIG. 18D shows the densitometry quantification of the Western blot data. ** P<0.01, *** P<0.001 (unpaired, two-tailed Student's t test).

FIGS. 19A, 19B, 19C, 19D, 19E, 19F, 19G, 19H, 19I, and 19J show the systemic neonatal administration of AAV9.BVES normalized muscle function and mass in BVES-KO mice. FIG. 19A shows the monthly body weight measurements of WT and BVES-KO mice with or without AAV9.BVES. FIG. 19B shows the representative photographs of WT and BVES-KO mice with or without AAV9.BVES at 9 months of age. FIG. 19C shows the tetanic torque measurements of the posterior compartment muscles of WT and BVES-KO mice with or without AAV9.BVES at 5 months of age. FIGS. 19D and 19E show the running time to exhaustion (FIG. 19D) and total running distance (FIG. 19E) of WT and BVES-KO mice with or without AAV9.BVES at 6 months of age on a 15° uphill treadmill. ** P<0.01; **** P<0.0001; ns, not significant. FIG. 19F show the representative images of dissected skeletal muscles from male WT and BVES-KO mice with or without AAV9.BVES. FIGS. 19G and 19H show the net mass of GA (FIG. 19G) and QU (FIG. 19H) in 9-month-old male mice with the designated genotype. FIGS. 19I and 19J show the relative mass of GA (FIG. 19I) or QU (FIG. 19J) muscle normalized to the body weight in 9-month-old male mice with the designated genotype.

FIGS. 20A, 20B, 20C, 20D, 20E, 20F, 20G, 20H, 20I, and 20J show the systemic adult administration of AAV9.BVES improved muscle function and mass in BVES-KO mice. FIG. 20A shows the biweekly body weight measurements of WT and BVES-KO mice with or without AAV9.BVES. FIG. 20B shows the representative photographs of WT and BVES-KO mice with or without AAV9.BVES at 9 months of age. FIG. 20C shows the tetanic torque measurements of the posterior compartment muscles of WT and BVES-KO mice with or without AAV9.BVES at 6 months of age. FIGS. 20D and 20E show the running time to exhaustion (FIG. 20D) and total running distance (FIG. 20E) of WT and BVES-KO mice with or without AAV9.BVES at 6 months of age on a 15° uphill treadmill. ** P<0.01. FIG. 20F shows the representative images of dissected skeletal muscles from male WT and BVES-KO mice with or without AAV9.BVES. FIGS. 20G and 20H show the net mass of GA (FIG. 20G) and QU (FIG. 20H) in 9-month-old male mice with the designated genotype. FIGS. 20I and 20J show the relative mass of GA (FIG. 20I) or QU (FIG. 20J) muscle normalized to the body weight in 9-month-old male mice with the designated genotype.

FIGS. 21A, 21B, 21C, 12D, and 21E show the systemic AAV9.BVES gene delivery improved the pathohistological defects in BVES-KO mice. FIG. 21A shows the H&E staining of GA muscle sections in WT and BVES-KO mice with or without AAV9.BVES at 9 months of age. Scale bar: 100 μm. FIG. 21B shows the immunostaining of MyHC-IIb and dystrophin (Dys) in GA muscles from WT and BVES-KO mice with or without AAV9.BVES. Scale bar: 100 μm. FIG. 21C shows the quantification of CNFs in the GA muscles of WT and BVES-KO mice with or without AAV9.BVES at 9 months of age. ** P<0.01; *** P<0.001; **** P<0.0001; ns, not significant. FIG. 21D shows the fiber size distribution of GA muscles from WT and BVES-KO mice with or without AAV9.BVES at 9 months of age. FIG. 21E shows the mean CSA of GA muscles from WT and BVES-KO mice with or without AAV9.BVES at 9 months of age. ** P<0.01; **** P<0.0001; ns, not significant.

FIGS. 22A, 22B, and 22C show the systemic AAV9.BVES gene delivery normalized ECG abnormalities in BVES-KO mice. FIG. 22A show the representative ECG data recorded from the male WT and BVES-KO mice with or without neonatal or adult administration of AAV9.BVES under the resting condition. FIGS. 22B and 22C show the heart rate (before or after exercise) of the male WT and BVES-KO mice with or without neonatal administration of AAV9.BVES. * P<0.05; ns, not significant (one-way ANOVA).

FIGS. 23A, 23B, 23C, and 23D show the safety profile of AAV9.BVES therapy in BVES-KO mice. FIGS. 23A, 23B, and 23C show the measurements of serum AST (FIG. 23A), ALT (FIG. 23B) and BUN (FIG. 23C) of WT and BVES-KO mice with or without neonatal or adult administration of AAV9.BVES at 9 months of age. FIG. 23D shows the H&E staining of liver sections from WT and BVES-KO mice with or without neonatal or adult administration of AAV9.BVES at 9 months of age. Scale bar: 100 μm.

FIGS. 24A, 24B, and 24C show the experimental design of neonatal and adult injection of AAV9.BVES into BVES-KO mice. FIG. 24A shows the schematic diagram of AAV9.BVES construct. FIGS. 24B and 24C show the timeline of AAV9.BVES treatment and analysis following neonatal (FIG. 24B) or adult (FIG. 24C) administration in male BVES-KO mice.

FIG. 25 shows the immunofluorescence staining images showing the expression of BVES transgene in BVES-KO mouse tissues. Representative, stitched immunofluorescence images of the entire heart, GA and liver sections stained with the anti-HA antibody from a 3-month-old BVES-KO mouse treated with AAV9.BVES at P3. Scale bars: 200 μm.

FIGS. 26A, 26B, 26C, 26D, and 26E show the impact of AAV9.BVES treatment on muscle function and mass in female BVES-KO mice after neonatal administration. FIG. 26A shows the representative images of female WT and BVES-KO mice treated with or without AAV9.BVES at 9 months of age. FIG. 26B shows the monthly body weight measurements of female WT and BVES-KO mice treated with or without AAV9.BVES. FIG. 26C shows the tetanic torque measurements of the posterior compartment muscles in female BVES-KO mice (5 months of age) treated with or without AAV9.BVES. FIGS. 26D and 26E show the running time to exhaustion (FIG. 26D) and total running distance (FIG. 26E) in 6-month-old female BVES-KO mice treated with or without AAV9.BVES determined by the treadmill running test. * P<0.05.

FIGS. 27A, 27B, and 27C show the western blot analysis of BVES transgene expression following tail vein injection of AAV9.BVES in adult male mice. Western blot showed AAV9.BVES was highly expressed in GA muscles (FIG. 27A) and heart (FIG. 27B) in BVES-KO mice with adult administration of AAV9.BVES. Arrows indicate the specific bands of BVES. The quantification was performed using ImageJ (FIG. 27C). * P<0.05, *** P<0.001.

FIG. 28 shows the serum CK measurements. Serum CK levels in WT and BVES-KO mice treated with or without AAV9.BVES at 9 months age. * P<0.05.

FIGS. 29A, 29B, and 29C show the effects of AAV9.BVES treatment on the heart rate of female BVES-KO mice. FIG. 29A shows the representative ECG recordings of female WT and BVES-KO mice treated with or without AAV9.BVES under the rest condition. FIGS. 29B and 29C show the average heart rate of female WT and BVES-KO mice treated with or without AAV9.BVES before (FIG. 29B) and after exercise (FIG. 29C). * P<0.05, ** P<0.01.

FIGS. 30A and 30B show the echocardiography measurement of cardiac function in BVES-KO mice with or without AAV9.BVES treatment. FIG. 30A shows the representative M-mode echocardiographic recording from 9-month-old WT and BVES-KO mice treated with or without AAV9.BVES. FIG. 30B shows the ejection fraction showed no significant changes among the three groups of mice.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand hybridizes under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs, or particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. In some embodiments, the promoter used herein is a tissue- or cell-specific promoter. In some embodiments, the promoter used herein is a muscle-specific promoter (e.g., MHCK7).

The term “recombinant” refers to a human manipulated nucleic acid (e.g., polynucleotide) or a copy or complement of a human manipulated nucleic acid (e.g., polynucleotide), or if in reference to a protein (i.e., a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell (e.g., a hepatocyte). The term “viral vector” is also meant to refer to those forms described more fully in U.S. Patent Application Publication U.S. 2018/0057839, which is incorporated herein by reference in its entirety. Suitable viral vectors include, e.g., adenoviruses, adeno-associated virus (AAV), vaccinia viruses, herpesviruses, baculoviruses and retroviruses, parvoviruses, and lentiviruses. In some embodiments, the viral vector is a lentiviral vector.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) means an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject. In some embodiments, the subject has a mutated BVES gene.

The term “about” as used herein when referring to a measurable value such as an amount, a percentage, and the like, is meant to encompass variations of ±20%, ±10%, ±5%, or ±1% from the measurable value.

“Therapeutically effective amount” or “therapeutically effective dose” of a composition refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The phrases “concurrent administration”, “administration in combination”, “simultaneous administration” or “administered simultaneously” as used herein, means that the compounds are administered at the same point in time or immediately following one another.

The term “biocompatible” generally refers to a material and any metabolites or degradation products thereof that are generally non-toxic to the recipient and do not cause significant adverse effects to the subject

The term “polypeptide” refers to a compound made up of a single chain of D- or L-amino acids or a mixture of D- and L-amino acids joined by peptide bonds.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Methods of Treating BVES-Related Diseases or Disorders

The present disclosure provides methods of treating, decreasing, reducing, and/or preventing progression of BVES-related diseases or disorders. The present disclosure also shows BVES functions as a negative regulator of ADCY9-mediated cAMP signaling. Disruption of BVES-mediated control of cAMP signaling leads to an increased protein kinase A signaling cascade, thereby promoting FoxO-mediated ubiquitin proteasome degradation pathway and autophagy initiation. It is demonstrated herein that a proteasome inhibitor, such as a selective inhibitor of 26S proteasome, can ameliorate the disease pathologies and improve muscle function in subjects with BVES-related disorders, including but not limited to muscular dystrophy and cardiac arrhythmia.

Accordingly, in some aspects, disclosed herein is a method of treating or preventing a blood vessel epicardial substance (BVES)-related disorder (such as muscular dystrophy or cardiac arrhythmia) in a subject in need thereof, comprising administering to the subject a therapeutically effective amount of an expression vector comprising a nucleic acid encoding a blood vessel epicardial substance (BVES) protein. In some embodiments, the nucleic acid is an RNA or a DNA.

In some embodiments, the nucleic acid encoding the BVES protein comprises a sequence at least 70% identical to SEQ ID NO: 1, or a fragment thereof. In some embodiments, the nucleic acid encoding the BVES protein comprises a sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1. In some embodiments, the nucleic acid encoding the BVES protein comprises SEQ ID NO: 1.

In some embodiments, the nucleic acid encoding the BVES protein is operably linked to a muscle-specific promoter. Herein, “operably linked” refers to the fusion of a first nucleic acid sequence, such as for example the nucleic acid encoding the BVES protein, and a second nucleic acid, such as for example the muscle-specific promoter. In some embodiments, the muscle-specific promoter includes, but is not limited to MHCK7, Desmin, Mb, alpha actin (α-actin), or other muscle-specific promoters.

In some embodiments, the MHCK7 promoter comprises a sequence at least 70% identical to SEQ ID NO: 3, or a fragment thereof. In some embodiments, the MHCK7 promoter comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 3, or a fragment thereof. In some embodiments, the MHCK7 promoter comprises SEQ ID NO: 3, or a fragment thereof.

In some embodiments, the nucleic acid encoding the BVES protein is operatively linked to an inverted terminal repeat (ITR) sequence. In some embodiments, the ITR sequence is at least 70% identical to SEQ ID NO: 4, or a fragment thereof. In some embodiments, the ITR sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 4, or a fragment thereof. In some embodiments, the ITR sequence comprises SEQ ID NO: 4, or a fragment thereof.

In some embodiments, the expression vector comprises a sequence at least 70% identical SEQ ID NO: 8, or a fragment thereof. In some embodiments, the expression vector comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 8, or a fragment thereof. In some embodiments, the expression vector comprises SEQ ID NO: 8, or a fragment thereof.

The present disclosure shows that adeno-associated virus 9 (AAV9)-mediated BVES gene transfer at neonatal or adult BVES-KO animals can prevent or alleviate the disease pathologies and improve the muscle mass and function in BVES-KO animals, supporting an AAV-based gene therapeutic approach for treating BVES deficient muscular dystrophy and cardiac arrhythmia.

In some embodiments, the expression vector is a viral vector. In some embodiments, the viral vector is an adeno-associated virus (AAV) vector. An adeno-associated virus (AAV) is a defective parvovirus that can infect many cell types and is nonpathogenic to humans. AAV type vectors can transport about 4 to 5 kb and wild type AAV is known to stably insert into chromosome 19. Vectors which contain this site specific integration property are preferred. Typically, the AAV coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV parvovirus.

In some embodiments, the AAV vector is an AAV1 vector, an AAV2 vector, an AAV3 vector, an AAV4 vector, an AAV5 vector, an AAV6 vector, an AAV7 vector, an AAV8 vector, or an AAV9 vector. In some embodiments, the AAV vector is an AAV vector of serotype rh9, rh10, rh74, AAVmyo, or MyoAAV.

In some embodiments, the subject has limb-girdle muscular dystrophy type R25 (LGMDR25). In some embodiments, the subject has cardiac arrhythmia.

In recent years, BVES/POPDC have been identified in the heart, smooth muscle, skeletal muscle, brain, liver, gastrointestinal (GI) tract, and various epithelia of humans. Thus, in some embodiments, the subject has a mutated BVES gene. In some embodiments, the subject has a dysfunctional BVES protein. In some embodiments, subject has a cardiovascular disease including, but not limited to coronary artery disease, high/low blood pressure, cardiac arrest/heart failure, congestive heart failure, congenital heart defects/diseases (including, but not limited to atrial septal defects, atrioventricular septal defects, coarctation of the aorta, double-outlet right ventricle, d-transposition of the great arteries, Ebstein anomaly, hypoplastic left heart syndrome, and interrupted aortic arch), arrhythmia, peripheral artery disease, stroke, cerebrovascular disease, renal artery stenosis, aortic aneurysm, cardiomyopathies, hypertensive heart disease, pulmonary heart disease, cardiac dysrhythmias, endocarditis, inflammatory cardiomegaly, myocarditis, eosinophilic myocarditis, valvular heart diseases, rheumatic heart diseases, and other related cardiovascular diseases. In some embodiments, the subject has a muscular disease/disorder including, but not limited to muscular dystrophy, polymyositis, dermatomyositis, myasthenia gravis, amyotrophic lateral sclerosis (ALS), rhabdomyolysis, cardiomyopathy, and sarcopenia.

In some embodiments, the method of any preceding aspect further comprises administering to the subject a therapeutically effective amount of a proteasome inhibitor. In some embodiments, the proteasome inhibitor is bortezomib, carfilzomib, or ixazomib.

The BVES vector alone or in combination with a proteasome inhibitor may be administered in such amounts, time, and route deemed necessary in order to achieve the desired result. The exact amount of the BVES vector alone or in combination with a proteasome inhibitor will vary from subject to subject, depending on the species, age, and general condition of the subject, the severity of the BVES-related disease/disorder, the particular vector composition, its mode of administration, its mode of activity, and the like. The BVES vector alone or in combination with a proteasome inhibitor is preferably formulated in dosage unit form for ease of administration and uniformity of dosage. It will be understood, however, that the total daily usage of the BVES vector alone or in combination with a proteasome inhibitor will be decided by the attending physician within the scope of sound medical judgment. The specific therapeutically effective dose level for any particular subject will depend upon a variety of factors including the BVES-related disease/disorder being treated and the severity of the symptoms; the activity of the BVES vector alone or in combination with a proteasome inhibitor employed; the specific vector and promoter employed; the age, body weight, general health, sex and diet of the patient; the time of administration, route of administration, and rate of excretion of the specific BVES vector alone or in combination with a proteasome inhibitor employed; the duration of the treatment; drugs used in combination or coincidental with the specific BVES vector alone or in combination with a proteasome inhibitor employed; and like factors well known in the medical arts.

The BVES vector alone or in combination with a proteasome inhibitor may be administered by any route. In some embodiments, the BVES vector alone or in combination with a proteasome inhibitor is administered via a variety of routes, including oral, intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the BVES vector alone or in combination with a proteasome inhibitor (e.g., its stability in the environment of the subject), the condition of the subject (e.g., whether the subject is able to tolerate administration), etc.

The exact amount of the BVES vector alone or in combination with a proteasome inhibitor required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is an expression vector of any preceding aspect and a pharmaceutically acceptable carrier selected from an excipient, a diluent, a salt, a buffer, a stabilizer, a lipid, an emulsion, a nanoparticle, and a cream. One or more active agents (e.g. the expression vector comprising the BVES nucleic acid) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the BVES vector alone or in combination with a proteasome inhibitor is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the BVES vector alone or in combination with a proteasome inhibitor is administered daily. In some embodiments, the BVES vector alone or in combination with a proteasome inhibitor is administered every day, every 2 days, every 3 days, every 4 days, every 5 days, every 6 days, every 7 days, or more. In some embodiments, the BVES vector alone or in combination with a proteasome inhibitor is administered every week, every 2 weeks, every 3 weeks, every 4 weeks, or more. In some embodiments, the BVES vector alone or in combination with a proteasome inhibitor is administered every month, every 2 months, every 3 months, every 4 months, every 5 months, every 6 months, every 7 months, every 8 months, every 9 months, every 10 months, every 11 months, every 12 months, or more. In some embodiments, the BVES vector alone or in combination with a proteasome inhibitor is administered every year, every 2 years, every 3 years, every 4 years, every 5 years, or more.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Defective BVES-Mediated Feedback Control of cAMP in Muscular Dystrophy

CAMP, synthesized by ADCYs and degraded by PDEs, is an important second messenger mediating the signaling cascade of numerous G protein-coupled receptors (GPCRs) involved in many aspects of muscle physiology, such as glycogenolysis, contractility, sarcoplasmic calcium dynamics, and muscle mass maintenance2. However, the feedback mechanisms for the CAMP signaling control in skeletal muscle are largely unknown. The Popeye domain containing (POPDC) genes encode a family of CAMP-binding proteins consisting of POPDC1 (commonly known as BVES), POPDC2 and POPDC3, and are abundantly expressed in skeletal muscle and heart. Genetic mutations in BVES were identified in patients with limb girdle muscular dystrophy type R25 (LGMDR25) and cardiac arrhythmia. Similar muscle and heart dysfunction was reported in mice, zebrafish, Xenopus and Drosophila models with BVES deficiency, indicating that BVES plays important, highly conserved functions in striated muscles. However, the molecular function of BVES remains to be determined.

### Bves Ablation Impairs Muscle Function and Exercise Performance in Mice

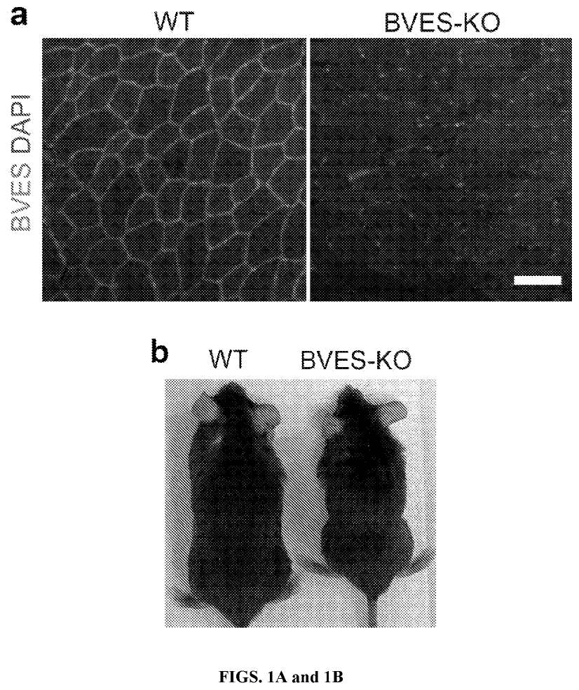

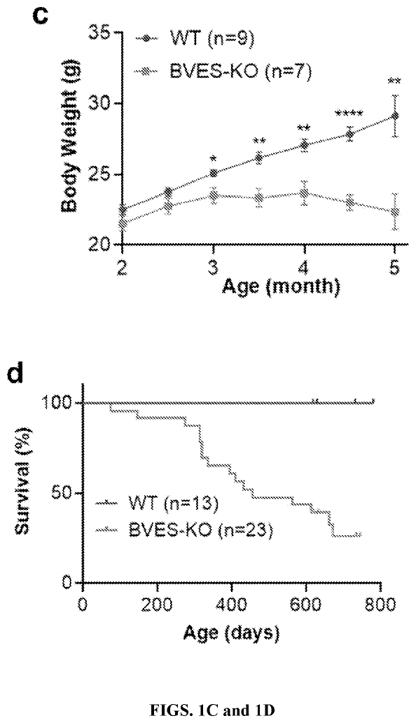

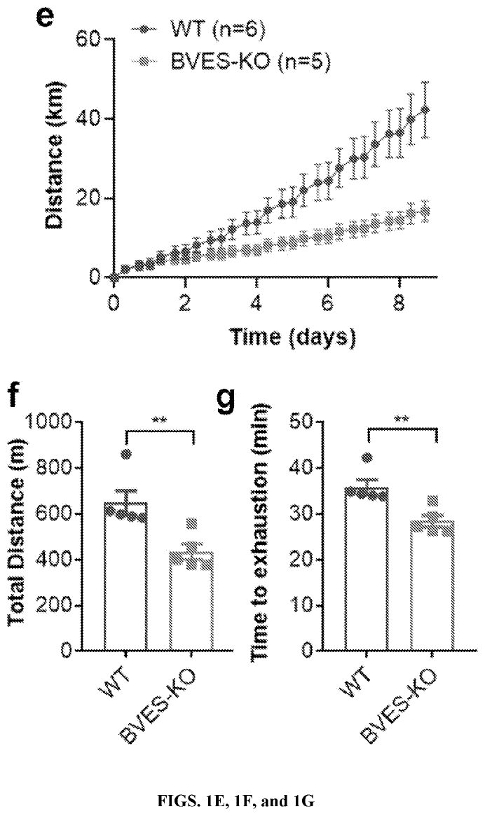

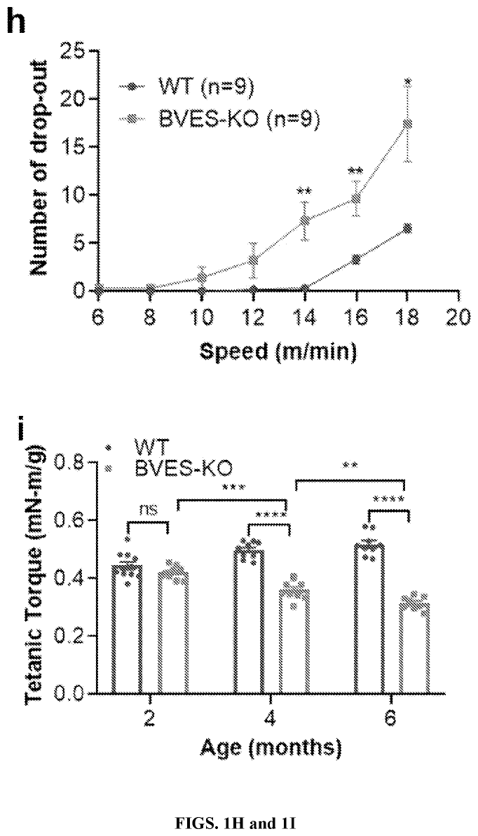

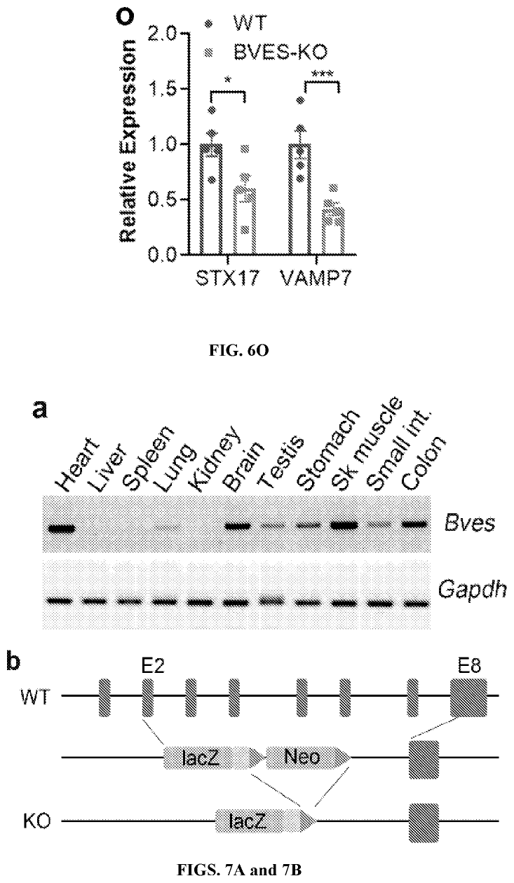

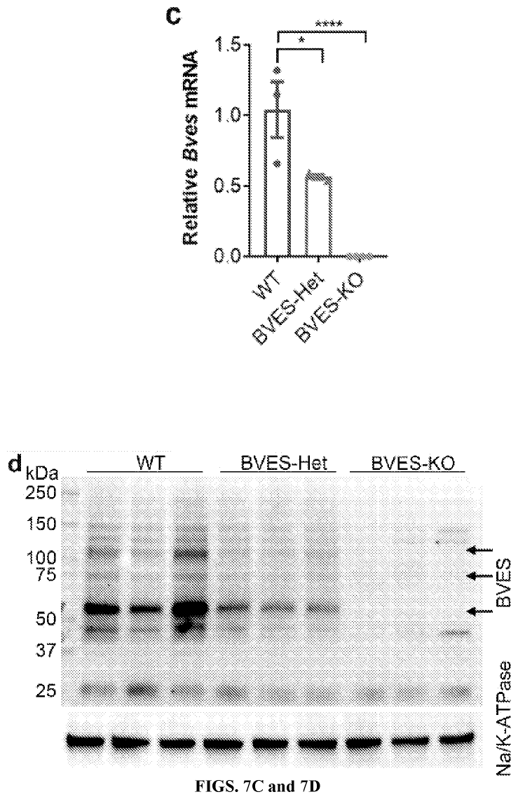

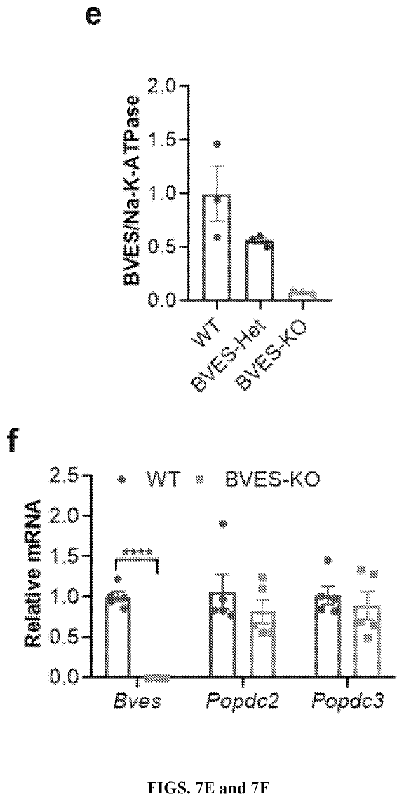

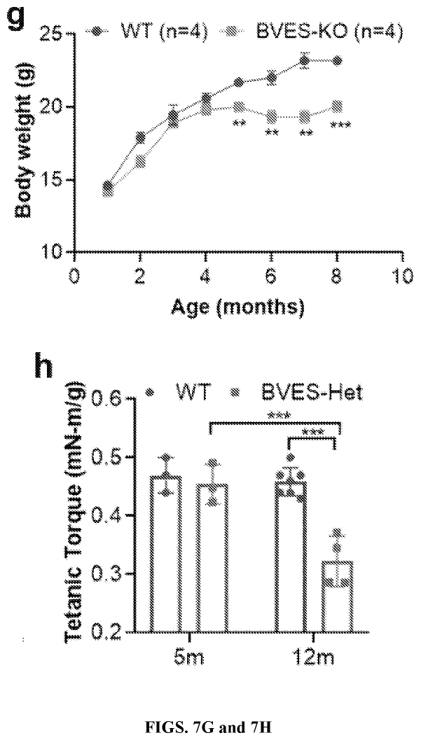

To investigate the physiological role of BVES in skeletal muscle, the expression of Bves in various mouse tissues was analyzed by RT-PCR and it was found that the Bves transcript was highly expressed in the striated muscle (FIG. 7A), consistent with previous reports. Next, a Bves knockout (BVES-KO) mouse line was established, in which the entire coding region of Bves spanning exon 2 to exon 8 were deleted (FIG. 7B). Immunostaining showed that BVES was mainly localized at the sarcolemma of wild-type (WT) muscle fibers and its expression was completely disrupted in the skeletal muscle of BVES-KO mice (FIG. 1A). Consistently, muscles of BVES-KO mouse showed remarkable decrease at both Bves transcript (FIG. 7C) and protein levels including monomeric, glycosylated and dimeric forms (FIGS. 7D and 7E). The transcript and protein expression of Bves were decreased by about 50% in the muscles of Bves heterozygous mice compared with WT (FIGS. 7D and 7E). However, the transcript expression of the other two members of the POPDC family including Popdc2 and Popdc3 was not significantly changed in skeletal muscles of BVES-KO mice (FIG. 7F).

The BVES-KO mice were fertile and smaller compared with the age/sex-matched littermate controls (FIG. 1B). The male BVES-KO mice showed a retarded growth from three months of age compared with the age/sex-matched WT mice (FIG. 1C). The female BVES-KO mice showed similar retarded growth but with a delayed onset (FIG. 7G). Kaplan-Meier survival curve revealed that the male BVES-KO mice had a reduced life span with 50% survival by around 60 weeks of age (FIG. 1D). To evaluate if Bves disruption affects the physical performance, the mice were subjected to voluntary wheel running for 9 consecutive days. As shown in FIG. 1E, the BVES-KO mice showed reduced running distance compared to WT littermate controls. Similarly, BVES-KO mice displayed a remarkable decrease in total running distance and the time to exhaustion in forced treadmill running test (FIGS. 1F and 1G). The BVES-KO mice displayed more dropouts, particularly at higher running speeds, than WT mice (FIG. 1H). To test if Bves disruption compromised the muscle function, the muscle contractility was measured using an in vivo muscle test system. Maximum plantarflexion tetanic torque was measured during supramaximal electric stimulation of the tibial nerve at 150 Hz. The BVES-KO mice exhibited progressive loss of muscle contractile strength starting from around 4 months of age (FIG. 1I). The heterozygous BVES-KO mice also displayed a significant loss of force production at 12 months of age but not at 5 months of age (FIG. 7H), indicating that haploinsufficiency of BVES compromises muscle function. Taken together, these results indicate that BVES plays an important role in maintaining muscle function.

### Bves Ablation Leads to Muscular Dystrophy and Atrophy

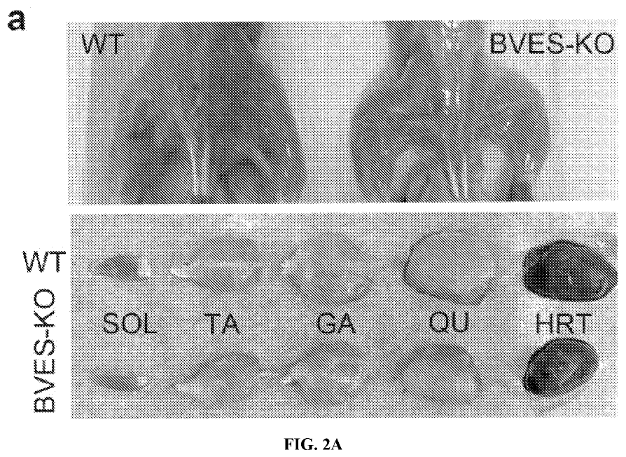

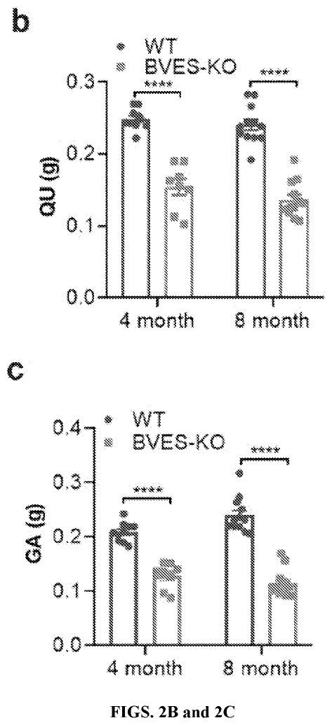

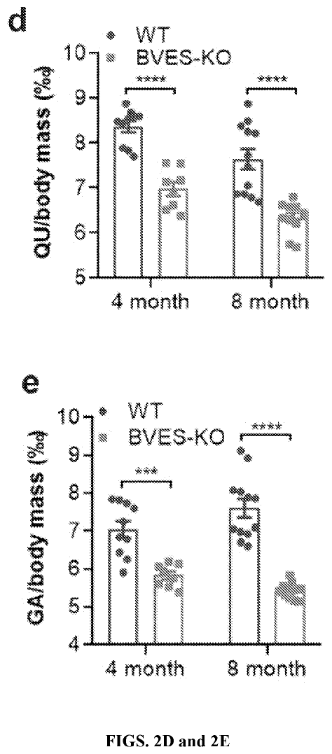

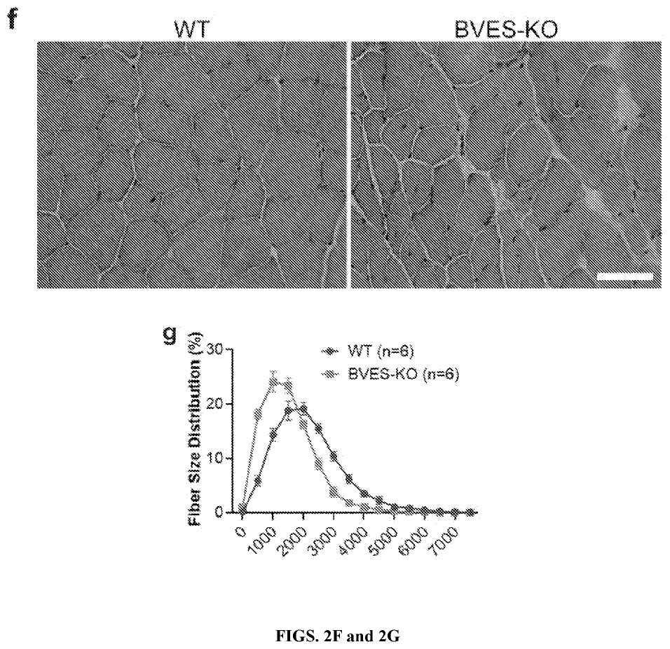

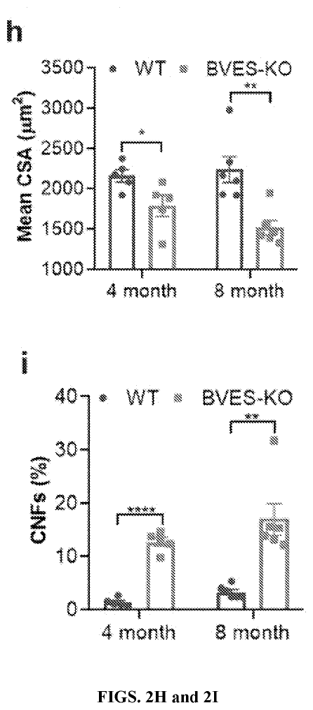

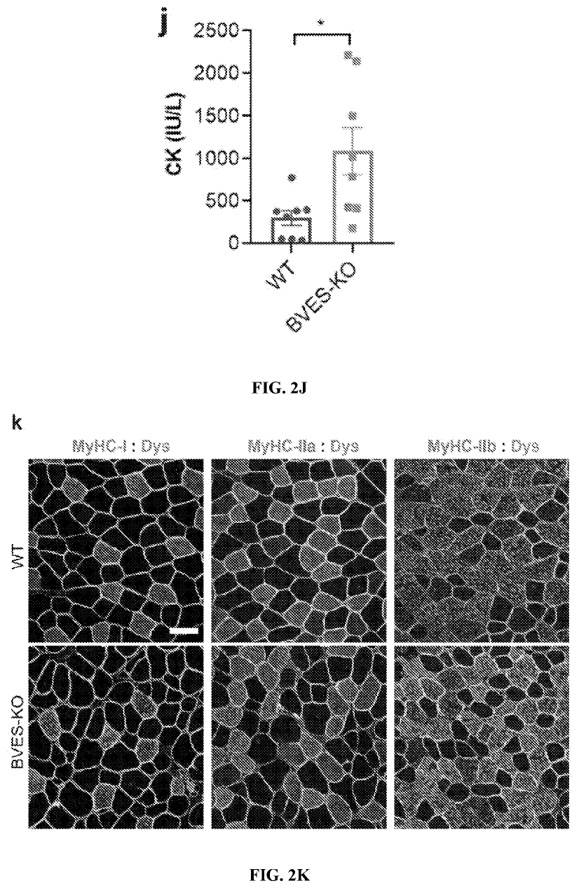

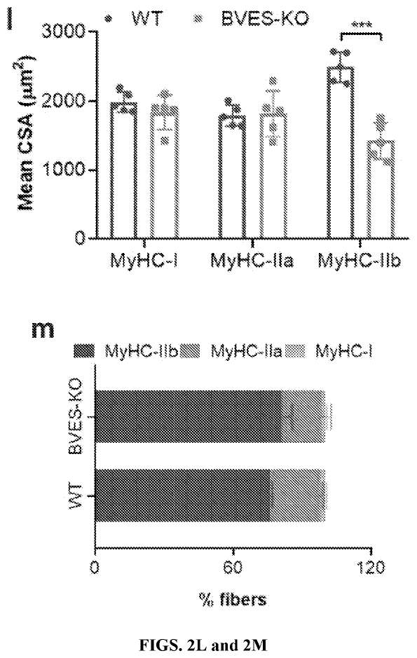

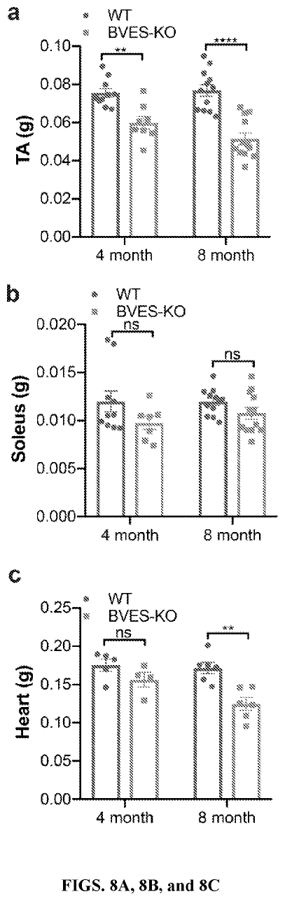

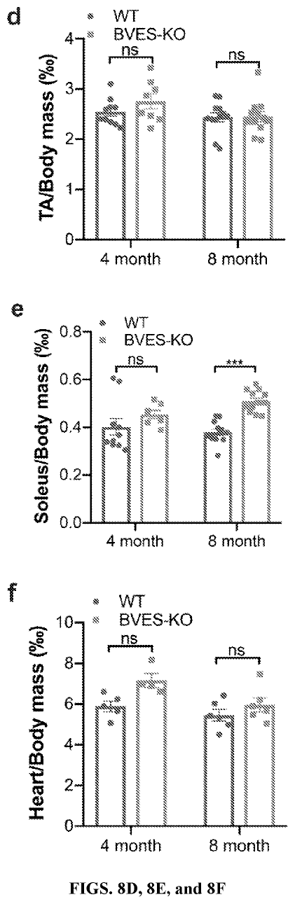

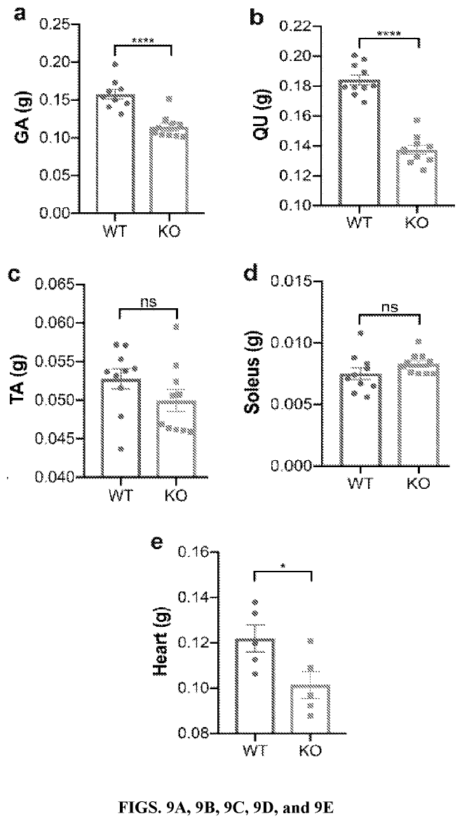

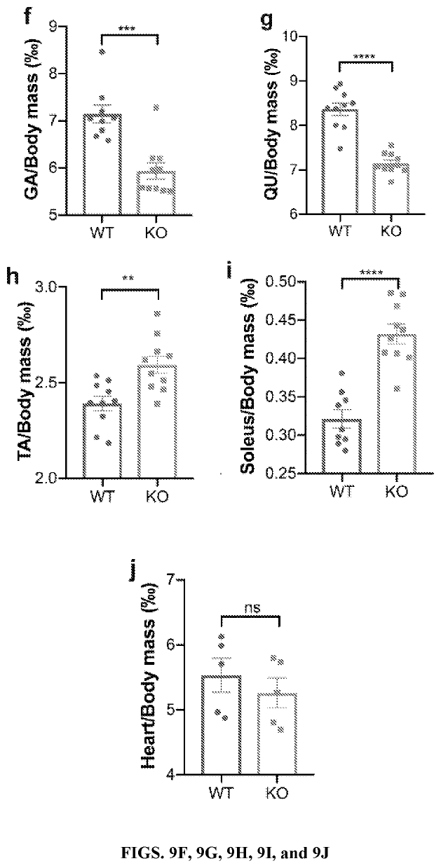

Next, histopathological analysis of skeletal muscle was performed in BVES-KO and WT mice. Reduced muscle mass was clearly visible in 4-month-old BVES-KO mice as compared to the age-matched WT littermate controls (FIG. 2A). At both 4 and 8 months of age, the mass of various skeletal muscles including gastrocnemius (GA), quadriceps (QU) and tibial anterior (TA) was significantly decreased (FIGS. 2B, 2C, and 8A), while the soleus muscle showed a trend of reduction (FIG. 8B). The heart mass was decreased in BVES-KO mice at 8 months of age (FIG. 8C). Since the BVES-KO mice were overall smaller than WT, the muscle mass was calculated and normalized to body weight. Again, it was found that the normalized muscle mass of GA and QU in BVES-KO mice was still significantly decreased as compared with WT littermates (FIGS. 8D and 8E) while the normalized mass of TA, soleus and heart showed no significant changes or slight increase in BVES-KO mice (FIGS. 8D and 8F). Similar results were observed in female mice (FIG. 9), indicating that the loss of BVES causes muscle atrophy regardless of gender.

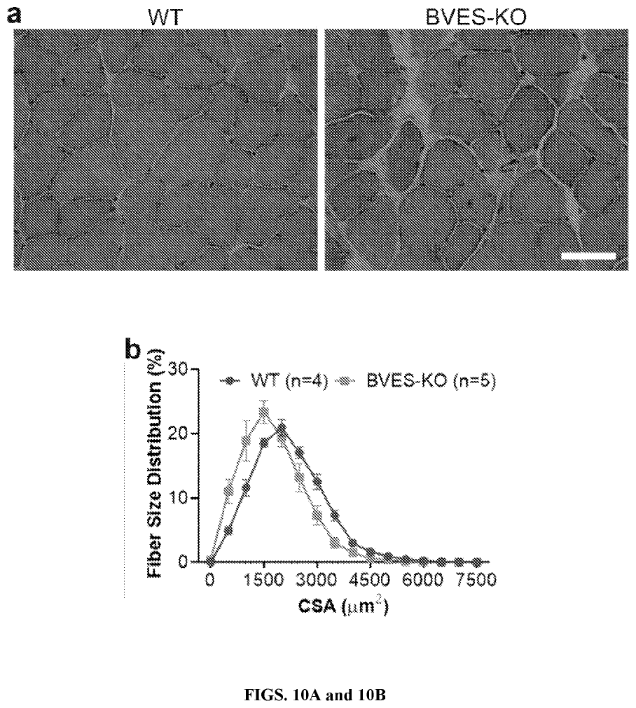

Muscle necrosis, centrally nucleated muscle fibers (CNFs) and angulated muscle fibers were readily observed in the H&E-stained sections of GA muscles from BVES-KO mice at 4 and 8 months of age (FIGS. 2F and 10). The fiber size distribution of the GA muscle shifted to the smaller fiber side in both 4- and 8-month-old BVES-KO mice compared with WT (FIGS. 2G and 10). The mean cross-sectional area (CSA) of muscle fibers were significantly reduced in both 4- and 8-month-old BVES-KO mice compared with WT (FIG. 2H). The percentage of CNFs increased to 12.8±0.8% and 17.0±3.0% in 4- and 8-month-old BVES-KO muscles, respectively (FIG. 2I). Serological analysis showed that the serum level of muscle creatine kinase was significantly elevated in BVES-KO mice compared with WT (FIG. 2J), consistent with previous reports in human patients with BVES mutations. These results indicate that muscular atrophy occurs along with dystrophy in BVES-KO muscles.

The impact of BVES deficiency on different types of muscle fibers was studied in GA muscle. Immunofluorescence staining was performed with antibodies against different isoforms of myosin heavy chain and dystrophin. As shown in FIGS. 2K and 2L, the type IIB muscle fibers were significantly smaller in the BVES-KO mice than those in WT mice while the type I and IIA muscle fibers were less affected. Moreover, there was a trend of increase in the percentage of type IIB muscle fibers while the type IIA muscle fibers showed a decrease trend (FIG. 2M), indicating a fiber type switching in BVES-KO mice.

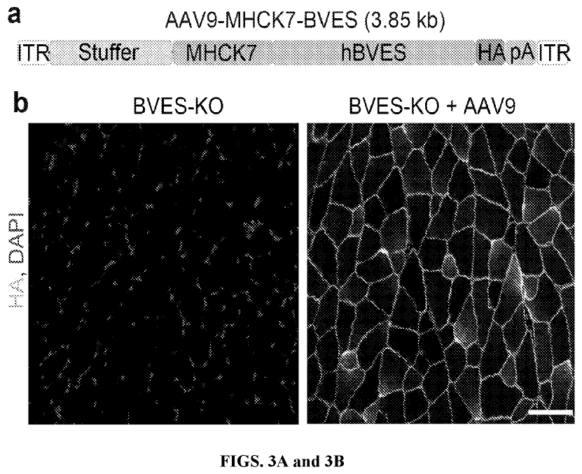

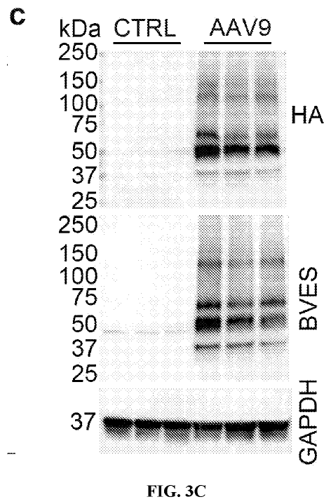

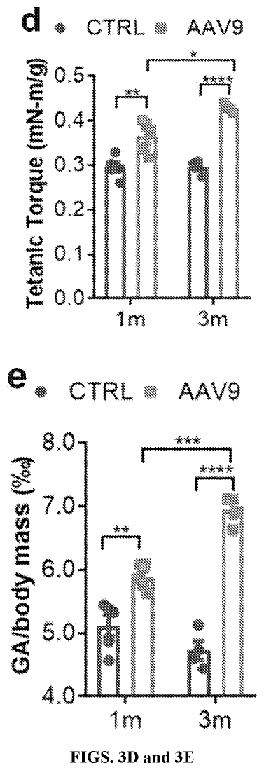

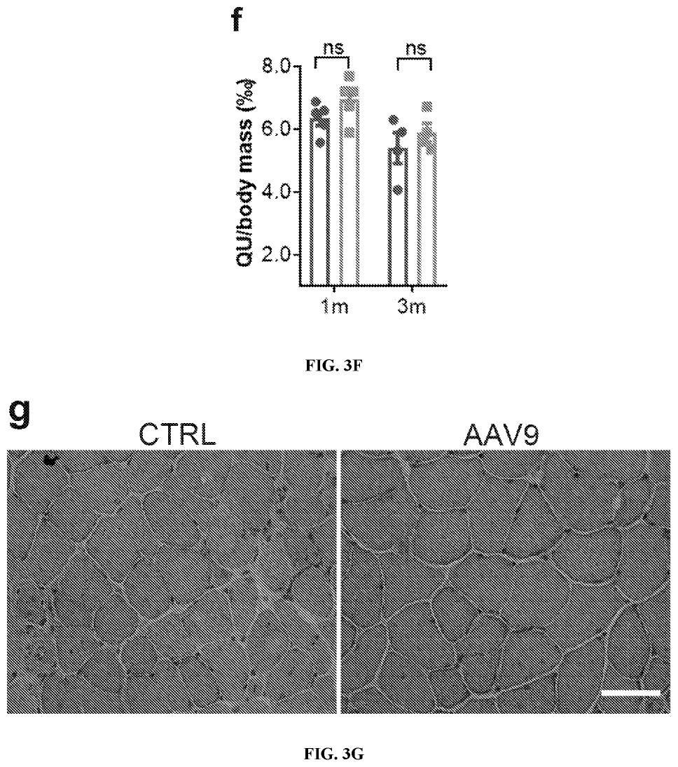

To test if the muscular dystrophy and atrophy in BVES-KO mice were due to the specific loss of BVES in skeletal muscle, a rescue experiment was performed with adeno-associated virus 9 (AAV9)-mediated gene transfer of BVES in skeletal muscle. This study generated an AAV9 carrying human BVES cDNA fused with the HA tag under the control of MHCK7 (FIG. 3A), a muscle-specific promoter active in mature skeletal muscle but not in muscle satellite cells. AAV9-BVES (2×10¹¹ vg) was injected into the GA muscles of 3-month-old BVES-KO mice. At one month after AAV9-BVES injection, immunofluorescence staining showed that almost all muscle fibers were positive for BVES (FIG. 3B). Similar to endogenous BVES (FIG. 1A), the BVES transgene expression was also mainly localized at the sarcolemma (FIG. 3B). Western blot analysis confirmed that BVES-HA transgene was highly expressed in the AAV9-BVES treated GA muscles but not in the contralateral GA muscles (FIG. 3C). Delivery of AAV9-BVES significantly increased muscle force production in BVES-KO mice at one month after injection and the rescue effect of AAV9-BVES on muscle force became more evident at three months (FIG. 3D). Moreover, AAV9-BVES delivery significantly increased the GA muscle mass by ˜15% at one month and ˜47% at three months after injection, compared with the contralateral untreated GA muscles (FIG. 3E). There was no significant change in the mass of the non-injected QU muscle (FIG. 3F). H&E staining showed that the muscle pathology was remarkably improved in the GA muscles treated with AAV9-BVES (FIG. 3G The muscle fiber size distribution was normalized (FIG. 3H), and the average muscle fiber size was increased by ˜19% following AAV9-BVES treatment (FIG. 3I). The percentage of CNFs was dramatically decreased in AAV9-BVES-treated GA muscles (FIG. 3J). These results indicate that the muscle pathology in the BVES-KO mice can be largely attributed to the loss of BVES expression in mature skeletal muscle fibers.

BVES Interacts with and Inhibits ADCY9-Mediated cAMP Signaling

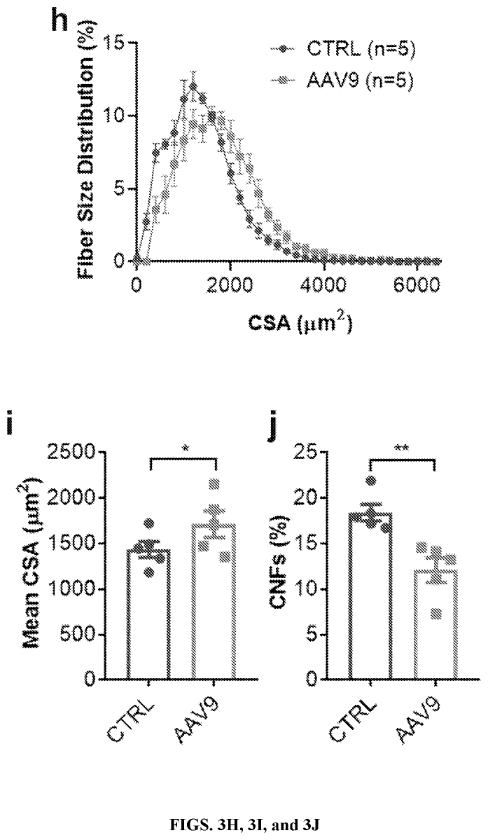

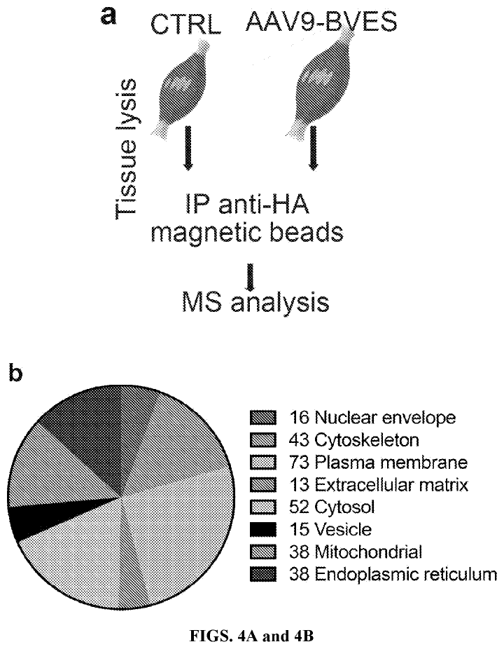

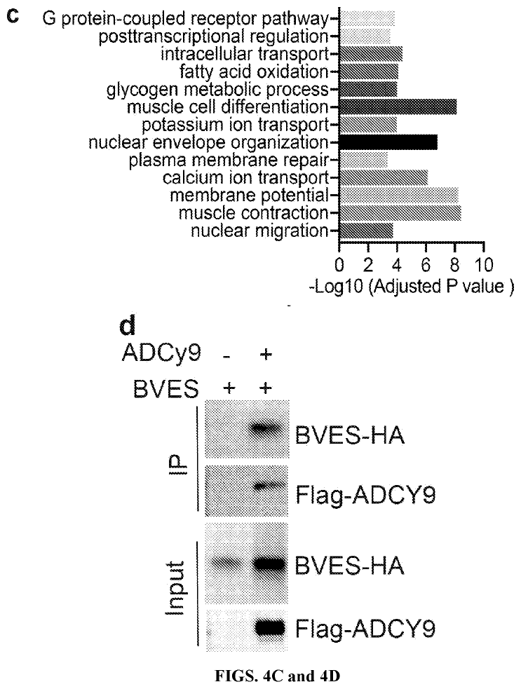

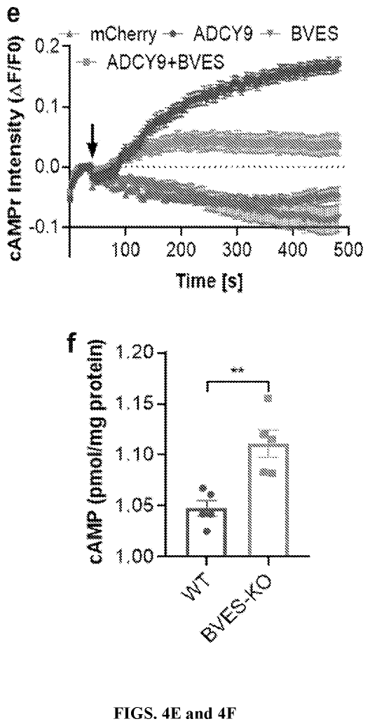

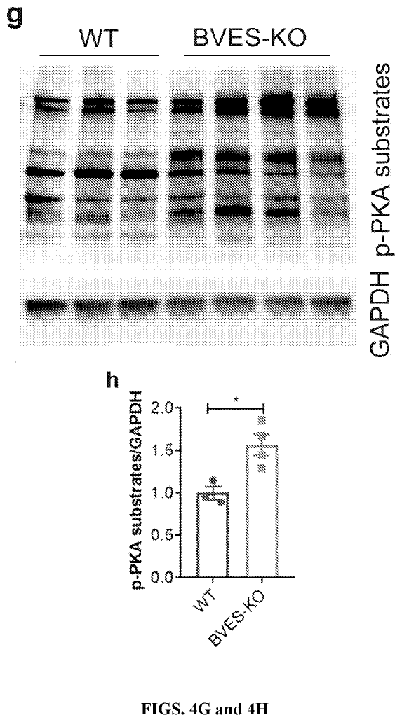

To understand the mechanism by which BVES deficiency leads to muscular dystrophy and atrophy, co-immunoprecipitation (co-IP) from AAV9-BVES-HA transduced skeletal muscle was performed using anti-HA antibody followed by mass spectrometry to identify the BVES-interacting proteins (FIG. 4A). After subtracting the background from a control sample, ˜186 putative BVES-interacting proteins were identified including some known interactors such as dysferlin and dystrophin (Table 1). Gene ontology (GO) analysis of subcellular localization found 73 candidates localized at the plasma membrane as the most highly enriched cellular compartment associated with BVES, 38 at endoplasmic reticulum (ER) and 16 at nuclear envelope, in concert with the fact that BVES was found at these subcellular compartments (FIG. 4B). GO analysis further showed that the BVES-interacting proteins are involved in a number of biological processes such as G protein-coupled receptor pathways, ion transport, membrane potential, muscle contraction, plasma membrane repair, muscle cell differentiation and nuclear envelope organization (FIG. 4C). ADCY9, a major adenylate cyclase responsible for cAMP biosynthesis in adult skeletal muscle, was identified among the BVES-interacting proteins (Table 1), which was further validated by co-IP with Flag-tagged ADCY9 and HA-tagged BVES in COS-1 cells (FIG. 4D).

The next experiment tested whether the CAMP-binding BVES can provide a feedback loop to regulate the cAMP biosynthesis via interacting with ADCY9. To test this, a mutant HEK293 cell line deficient in ADCY3 and 6 (the two major ADCY genes expressed in HEK293 cells) was first generated using cytosine base editing (CBE) (FIG. 11), as a homologous reconstitution system to probe the function of exogenously expressed ADCY9. The ADCY3/6-mutant HEK293 cells were also stably transduced with cAMPr, a genetically engineered fluorescent sensor of cAMP. Measurement of the cAMPr fluorescence in the ADCY3/6-mutant HEK293 cells showed that inhibition of PDEs with a non-selective PDE inhibitor 3-isobutyl-1-methylxanthine (IBMX) induced a gradual increase of CAMP in ADCY9-transfected cells but not in the mCherry-transfected cells (FIG. 4E), indicating that the exogenously transfected ADCY9 contributed to the biosynthesis of CAMP in this mutant cell line. Co-transfection with BVES and ADCY9 together resulted in a substantially reduced cAMP elevation in response to IBMX inhibition (FIG. 4E), indicating that BVES inhibits ADCY9's activity. Consistently, the CAMP levels in the skeletal muscles were significantly increased in BVES-KO mice as compared to WT mice (FIG. 4F).

Elevation of cAMP activates PKA to mediate signal amplification. Western blot showed the phosphorylation of PKA substrates was significantly increased in BVES-KO mice (FIGS. 4G and 4H). Moreover, Western blot analysis of the downstream signaling of PKA revealed that phosphorylation of the PKA substrate LKB1 (liver kinase B1) and its primary target adenosine monophosphate (AMP)-activated protein kinase (AMPK) were significantly increased in BVES-KO muscles (FIGS. 4I-4K). These data indicate that the loss of BVES-mediated negative regulation of CAMP signaling leads to an increased PKA-dependent signaling cascade.

### Dysregulated PKA Signaling Leads to Activation of the Ubiquitination-Proteasome Degradation System (UPS) in BVES-Deficient Skeletal Muscle

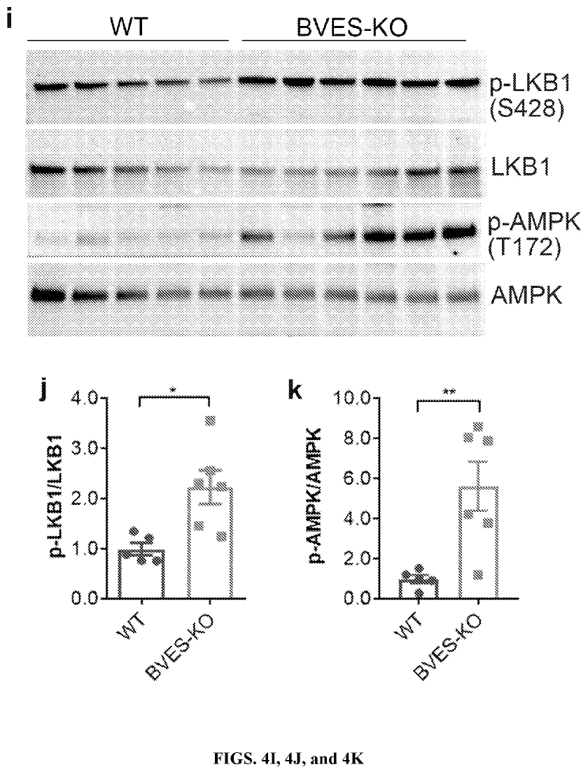

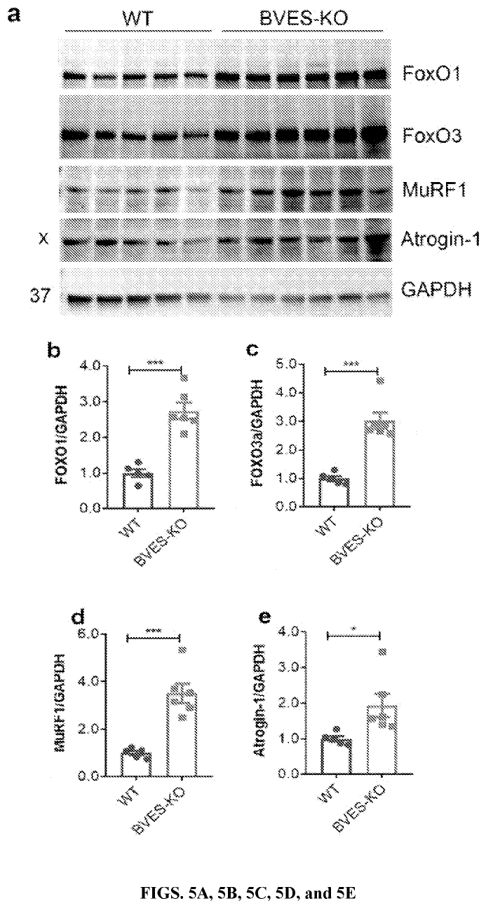

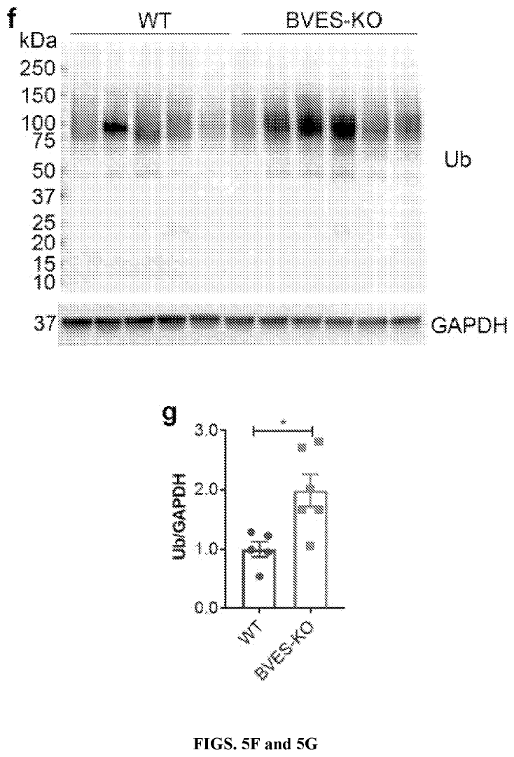

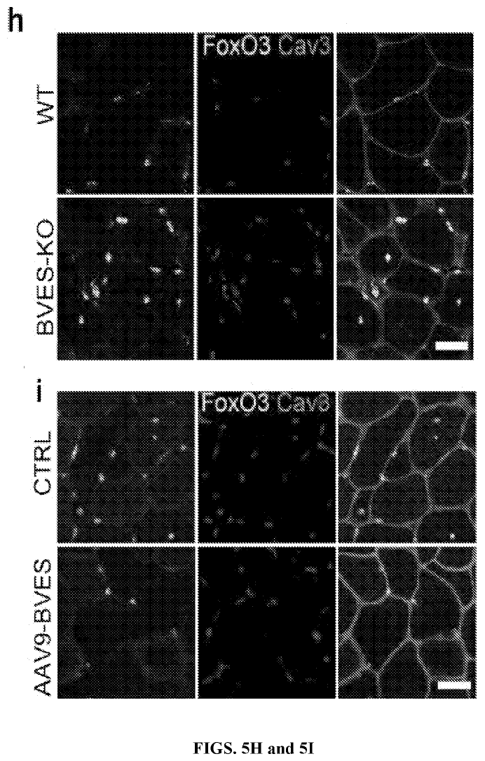

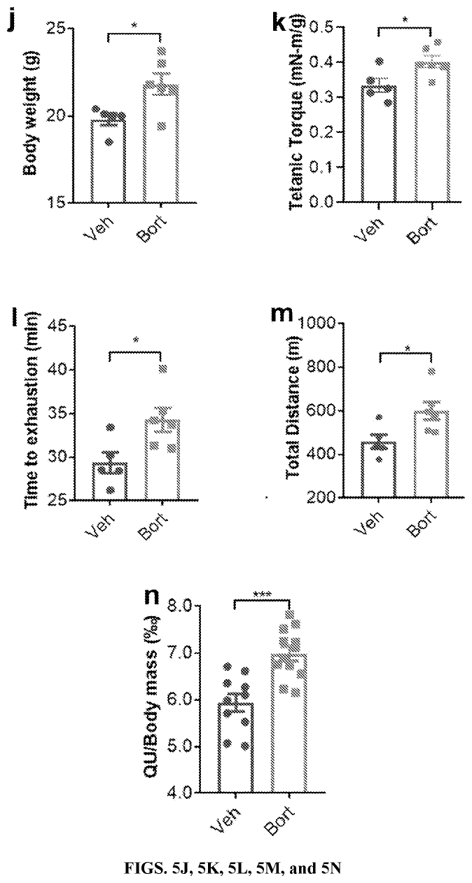

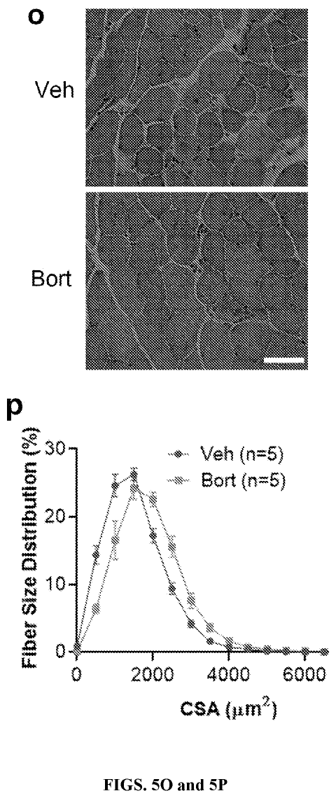

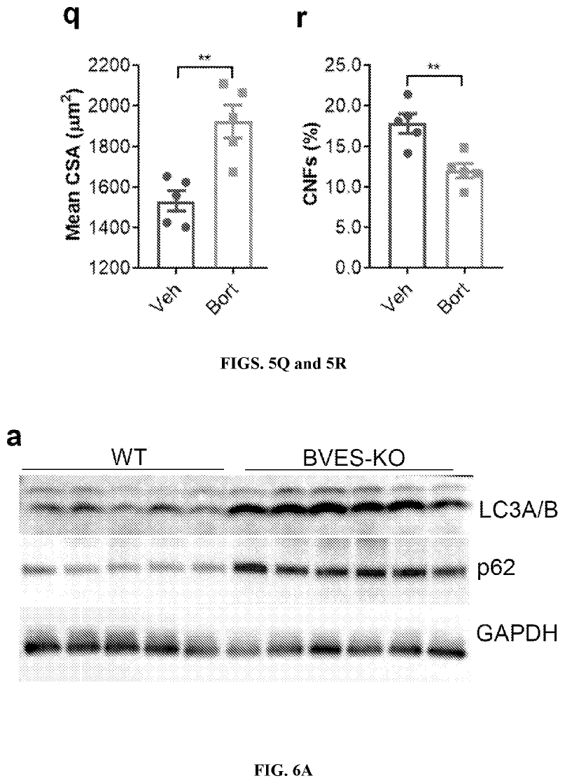

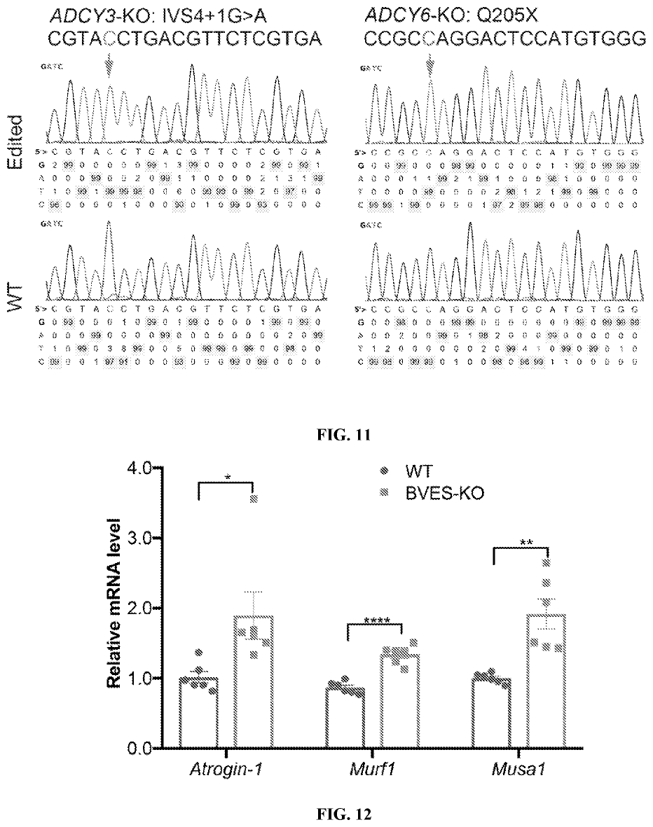

Previous studies showed that AMPK activation promotes forkhead box (FoxO)-mediated UPS degradation, which can contribute to the muscle pathology in BVES-KO mice. Consistently, it was found that the expression of FoxO1 and FoxO3a were significantly increased by over 2 folds in BVES-KO skeletal muscle compared with WT controls (FIGS. 5A, 5B, and 5C). Similarly, the FoxO-regulated ubiquitin E3 ligases Atrogin-1 and MuRF1 were also significantly upregulated at both the transcriptional (FIG. 12) and protein levels in BVES-KO muscles (FIGS. 5A, 5D, and 5E). Moreover, the global protein ubiquitination was significantly increased in BVES-KO muscles (FIGS. 5F and 5G), indicating that BVES disruption led to enhanced UPS activation. Given that the nuclear translocation was essential for FoxO to function as transcription factors, FoxO3 was significantly increased in the nuclei of BVES-KO skeletal muscles compared with WT (FIG. 5H). Conversely, AAV9-BVES gene delivery decreased FoxO3a in the nuclei of BVES-KO skeletal muscles (FIG. 5I). Thus, these data indicate that FoxO is involved in the activation of UPS in BVES-deficient skeletal muscle.

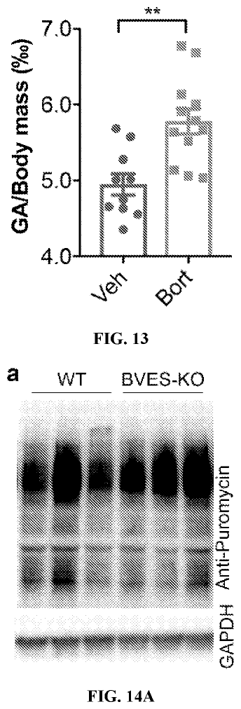

To further investigate the role of UPS in the pathogenesis of BVES-deficient muscular dystrophy and atrophy, the effect of bortezomib, a selective inhibitor of 26S proteasome approved by the U.S. Food and Drug Administration (FDA) to treat certain types of cancer such as multiple myeloma and mantle cell lymphoma, was evaluated on the muscle function and pathology in BVES-KO mice. Bortezomib treatment significantly increased the body weight (FIG. 5J), enhanced the muscle contractility (FIG. 5K) and improved physical performance on treadmill running in BVES-KO mice (FIGS. 5L and 5M). Moreover, bortezomib treatment significantly increased the mass of quadriceps and gastrocnemius muscles in BVES-KO mice (FIGS. 5N and 13). H&E staining showed that bortezomib treatment remarkably improved the muscle pathology as evidenced by more evenly organized muscle fibers (FIG. 5O). Bortezomib also significantly increased the fiber size (FIGS. 5P and 5Q) and reduced the percentage of CNFs (FIG. 5R). Taken together, bortezomib significantly improved muscle function and ameliorated the histopathology of skeletal muscles in BVES-KO mice.

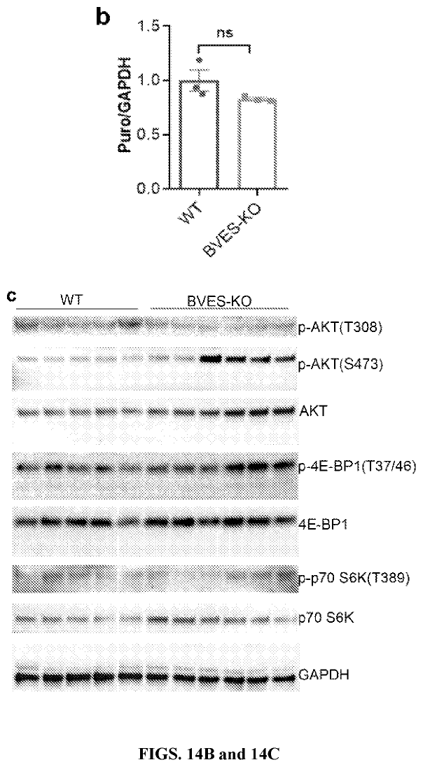

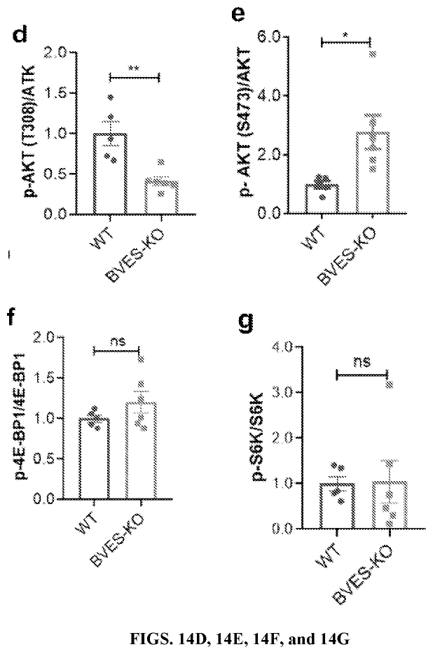

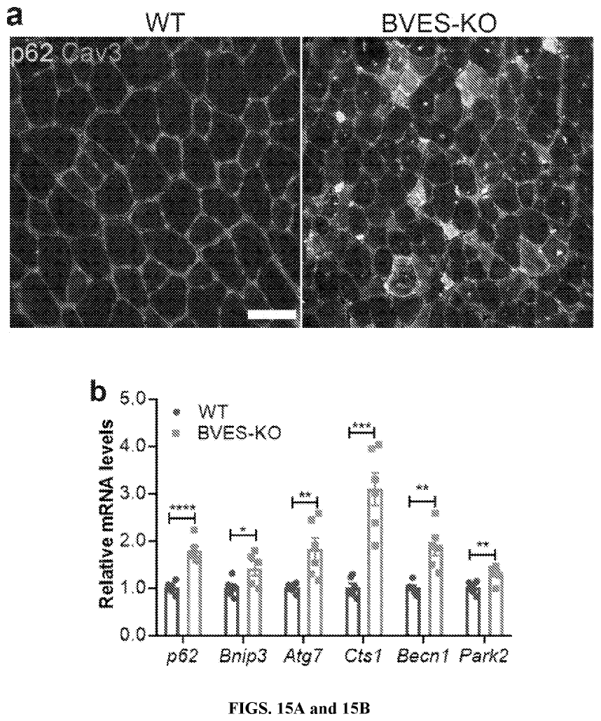

As muscle mass is determined by the balance between protein synthesis and degradation, this study also examined if BVES deficiency impacts the protein synthesis using the SUNSET assay. No significant changes were found in the total protein synthesis in BVES-KO skeletal muscles (FIGS. 14A and 14B). The AKT/mTOR signaling pathway was also examined related to protein translation. It was well known that phosphorylation of Thr308 (controlled by PDK1) and Ser473 (controlled by mTORC2) residues is required for maximal activation of AKT. It was observed that Thr308 phosphorylated AKT was significantly decreased while S473 phosphorylation was increased in BVES-KO muscles (FIGS. 14C, 14D, and 14E). Furthermore, phosphorylation of both 4E-BP1 (Thr 37/46) and S6K (Thr389), the two downstream targets of mTOR that are responsible for protein translation, was similar between WT and BVES-KO skeletal muscles (FIGS. 14C, 14F, and 14G). These data indicate that the protein biosynthesis governed by the AKT/mTOR axis was minimally affected in BVES-KO mice.

### BVES Deficiency Compromises Autophagy Execution

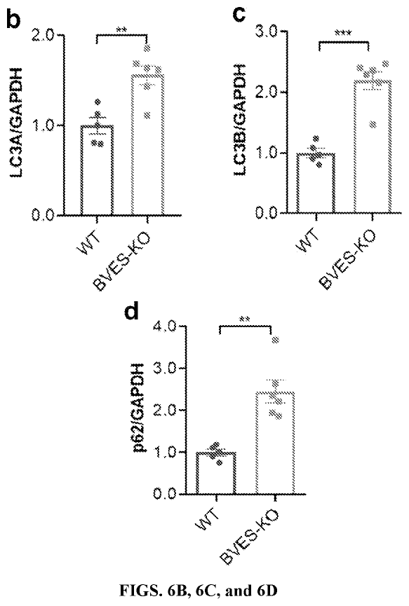

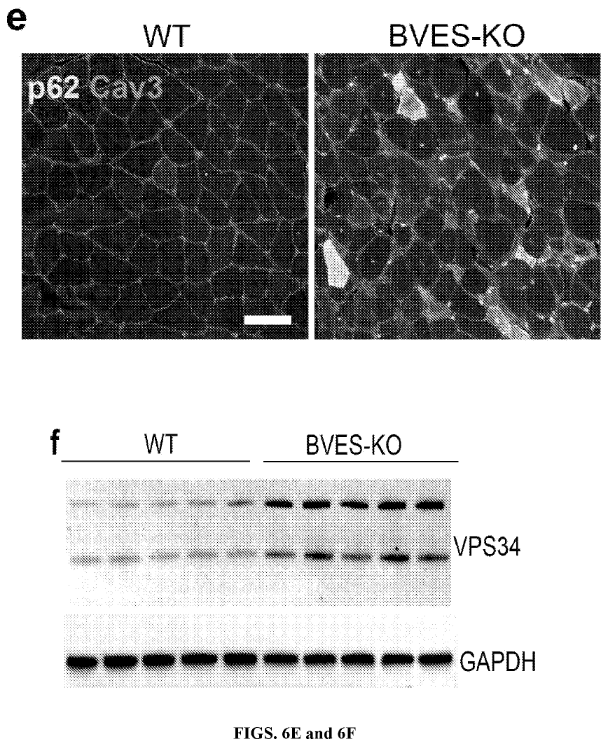

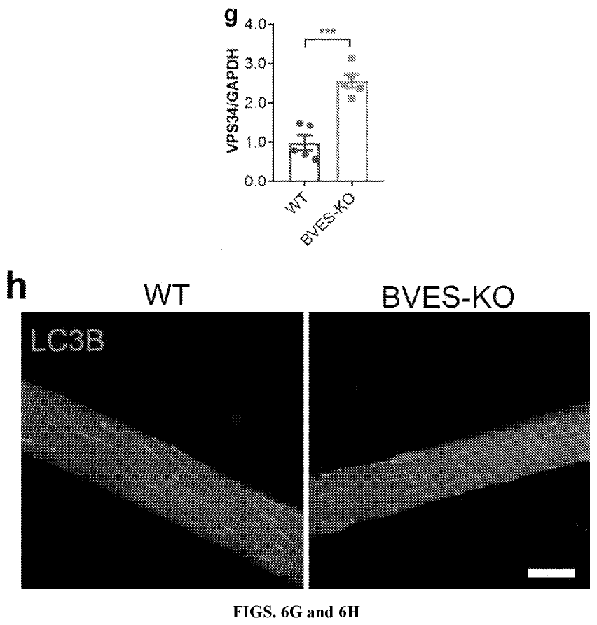

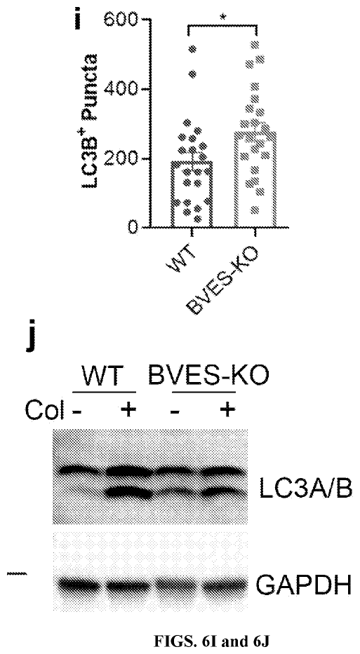

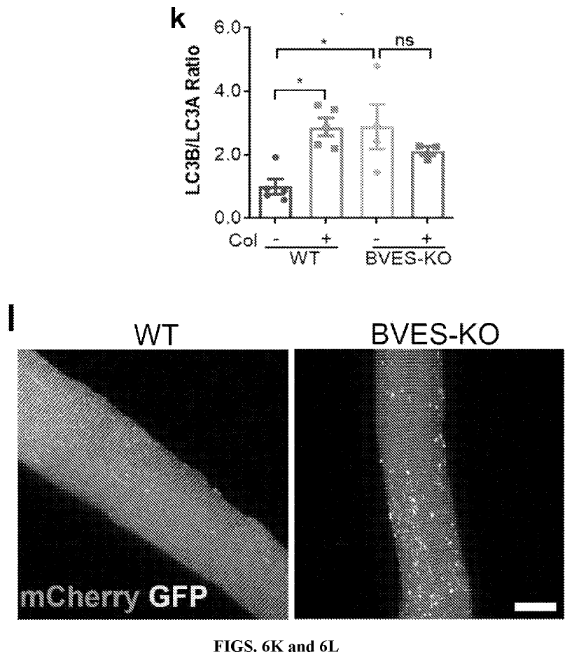

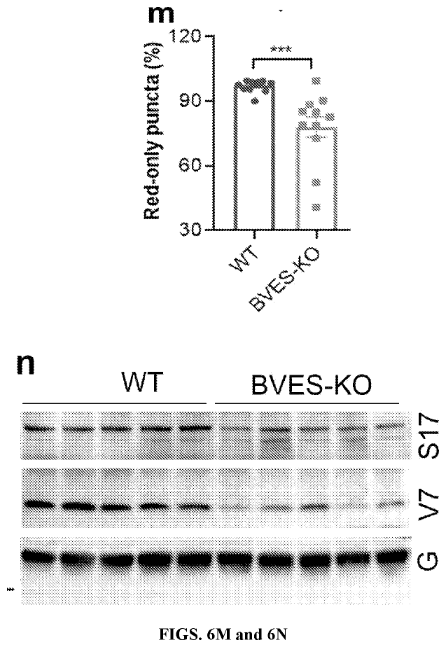

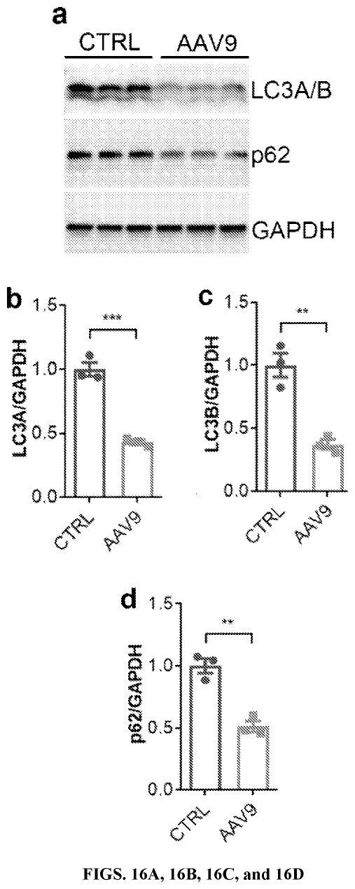

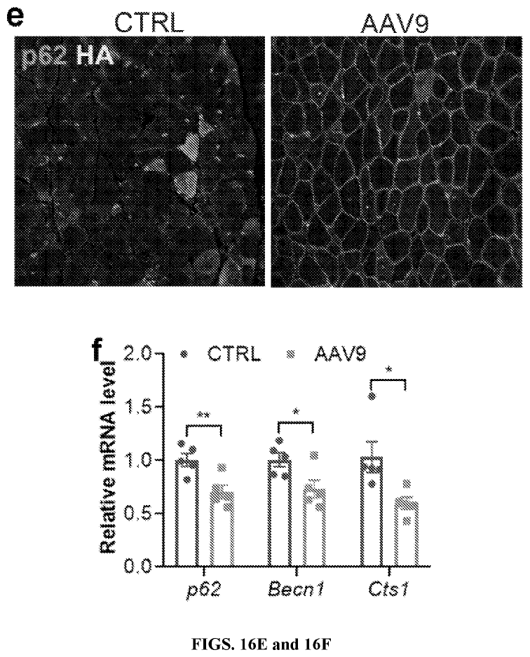

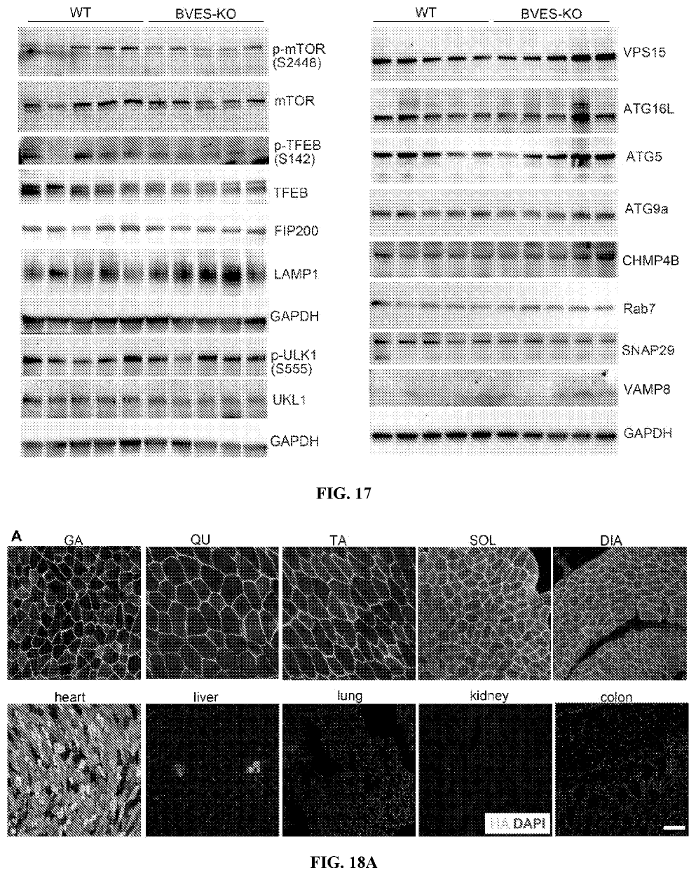

Since FoxO signaling also regulates autophagy-related genes, it can be reasoned that BVES disruption can alter the autophagy process. Western blot detected a significant upregulation of the autophagy markers LC3A, LC3B and p62 in the BVES-KO muscles (FIGS. 6A, 6B, 6C, and 6D). Likewise, immunostaining showed that p62 was accumulated in the BVES-KO skeletal muscles (FIGS. 6E and 15A). The transcript expression levels of the genes related to autophagy initiation such as p62, Bnip3, Atg7, Cts1, Beclin-1 and Park2 were also significantly increased in the BVES-KO muscles (FIG. 15B), in line with the notion that their transcription is activated by the FoxO pathway. Conversely, AAV9-mediated delivery of BVES reversed the aberrant changes in autophagy initiation associated with BVES deficiency (FIG. 16). Furthermore, live cell imaging showed more RFP-LC3B+ puncta in the flexor digitorum brevis (FDB) muscle fibers of BVES-KO mice (FIGS. 6H and 6I) Autophagy initiation is orchestrated by a number of protein complexes including the core negative regulator of autophagy (mTOR complex), ULK1 initiation complex (ULK1, FIP200, etc.), PI3K III nucleation complex (VPS15, VPS34, etc.), PI3P-binding complex (ATG16L, ATG5, etc.) and the protein related with lipid delivery (ATG9a). It was found that VPS34 was dramatically increased in the BVES-KO muscles (FIGS. 6F and 6G) while other proteins tested were unchanged (FIG. 17).

To further understand how BVES ablation regulates the dynamic autophagy process, the next experiment examined autophagic flux in WT and BVES-KO skeletal muscles using colchicine, which blocks the fusion of autophagosome with lysosome. A significant increase of LC3B was observed upon colchicine treatment in WT muscles but not in BVES-KO muscles (FIGS. 6J and 6K), indicating that BVES disruption suppresses the autophagic flux. This was corroborated by the live cell imaging study using the dual fluorescent reporter mCherry-GFP-LC3B in FDB muscles of BVES-KO and WT mice. As shown in FIGS. 6L and 6M, the percentage of the autolysosomes (indicated by the red-only puncta) was significantly reduced in BVES-KO mice.

The increased autophagy initiation and reduced autolysosome formation indicate that the fusion of autophagosome with lysosome is inhibited in the BVES-KO muscles. Western blot was performed to analyze the expression of some key proteins involved in the autophagic fusion such as the STX17, Rab7, VAMP7 and VAMP8. Interestingly, STX17 and VAMP7, but not the other proteins measured, were dramatically decreased by ˜50% in BVES-KO muscles (FIGS. 6N, 6O, and 17), indicating that the suppression of autophagy flux can be due to downregulation of the SNARE proteins in BVES-KO skeletal muscles.

In summary, this work unveils a novel role of BVES in providing a negative feedback control for ADCY9 to regulate the CAMP signaling in skeletal muscle. The loss of BVES-mediated feedback control of the CAMP signaling promotes PKA-mediated signaling cascade, dysregulates protein quality control systems and eventually leads to the loss of muscle integrity and function, highlighting the importance of this feedback control mechanism of CAMP in skeletal muscle. The different subcellular localization of BVES39 indicates that BVES can offer a feedback control mechanism for spatialized cAMP regulation. Because CAMP signaling participates in various physiological processes in many different types of mammalian cells beyond skeletal muscle, defects in this feedback control mechanism can be linked to other human diseases.

In support of this finding that BVES inhibits ADCY9's activity, the BVES knockout mouse hippocampal neurons was found to display PKA-dependent enhancement of long-term potentiation. The question remains as to how BVES regulates the activity of ADCY9. BVES can inhibit ADCY9's activity in a cAMP concentration-dependent manner through the Popeye domain-mediated interaction. This is supported by a recent preprint at biorxiv showing that BVES interacts with ADCY9 via both the transmembrane domain and the C-terminal Popeye domain, although the authors did not study the regulation of ADCY9's activity by BVES. The bimolecular fluorescence complementation (BiFC) assay showed that the Popeye domain interacted with ADCY9, but the co-IP failed to detect this interaction, indicating that the interaction between the Popeye domain and ADCY9 is likely transient in nature and can depend on the CAMP binding status. Another mechanism for BVES-mediated inhibition of ADCY9 is to regulate the membrane tracking of ADCY9 in cAMP-dependent manner. In support of this, BVES was shown to play a role in membrane protein trafficking. Moreover, BVES was reported to interact with PDE4, indicating that BVES can provide a delicate regulation of cAMP signaling through multiple interactions with the cAMP signaling machinery. Finally, it remains to be determined whether the structurally related BVES homologs POPDC2 and POPDC3 coordinate with BVES or independently regulate different ADCY proteins. Given the significance of genetic variations in BVES, POPDC2 and POPDC3 to human health, it can be important to examine how mutations in these genes contribute to muscular dystrophy, cardiac arrhythmia and other conditions, in regard to the dysregulated cAMP signaling, and determine whether restoring the CAMP signaling could rescue the disease pathologies associated with their genetic defects.

These data demonstrated that the loss of BVES-mediated CAMP feedback mechanism led to aberrant activation of UPS via a PKA-LKB1-AMPK-FoxO axis and treatment with the proteasome inhibitor bortezomib partially alleviated the muscle pathologies in BVES-KO mice, highlighting the therapeutic targeting of the cAMP/PKA-UPS signaling cascade for the treatment of BVES-related diseases. Herein, it was also revealed that BVES deficient skeletal muscle displayed a confound defect in autophagy execution. Previous studies showed that CAMP/PKA can either inhibit or activate autophagy depending on the cell or tissue context. Exactly how BVES deficiency causes the autophagic defects and the relationship between the cAMP/PKA signaling and autophagy execution in skeletal muscle remain to be investigated. These data showed that downregulation of syntaxin 17 and VAMP7, which are involved in autophagosome fusion, contributes to the observed defects in autophagy associated with BVES-KO skeletal muscles.

### Materials and Methods

Mice. All animal studies were reviewed and approved by the Institutional Animal Care and Use Committee (IACUC) of the Ohio State University. C57BL/6N mice were purchased from The Jackson Laboratory (Bar Harbor, ME). The BVES-KO mice (C57BL/6N-Bvestm1.1 (KOMP) Vlcg/MbpMmucd) with all coding exons deleted were obtained from Mutant Mouse Resource & Research Centers, UC Davis and maintained in a barrier facility. The BVES-KO mice were genotyped by PCR analysis of genomic DNA prepared from ear clips with the following primers. The KO allele was amplified with a forward primer 5′-ACTTGCTTTAAAAAACCTCCCACA (SEQ ID NO: 9) and a reverse primer 5′-AGTCACTAGCAAGAGATCTGCACCC (SEQ ID NO: 10) and the WT allele was amplified using a forward primer 5′-AAGTGCTGGGATTAAAGGTGTGTGC (SEQ ID NO: 11) and a reverse primer 5′-AAGGACACATCACAGCTTCAGG (SEQ ID NO: 12). The WT and KO allele would produce a 164-bp and 771-bp band, respectively.

Plasmids. The pX601-stuffer-MHCK7-BVES-3×HA plasmid was constructed by subcloning the BVES fragment amplified from BVES-myc and stuffer sequence from pLenti-hANO5WTint6BioID2 into Xhol and NotI digested pX601-AAV-CMV::NLS-SaCas9-NLS-3×HA-bGHpA;U6::BsaI-sgRNA, a gift from Feng Zhang (Addgene #61591). The pLVX-BVES-3×HA-puro was constructed by subcloning the BVES-3×HA into pLVX-puro (Clontech, San Jose, CA). The pCMV3-ADCY9-FLAG plasmid was purchased from Sino Biological (#HG19950-CF, Wayne, PA). pLVX-cAMPr-puro was constructed by subcloning the cAMPr fragment amplified from p2lox-cAMPr, a gift from Justin Blau (Addgene #99143) into pLVX-puro. pCMV-AncBE4max was a gift from David Liu (Addgene #112094). The annealed gRNA oligos (targeting human ADCY3 and ADCY6) were cloned into pLenti-OgRNA-Zeo plasmid as previously described. pDEST-CMV-mCherry-GFP-LC3B WT was a gift from Robin Ketteler (Addgene #123230). pmRFP-LC3 was a gift from Tamotsu Yoshimori (Addgene #21075).

Cell culture and transfection. Cos1 and HEK293 cell lines were obtained from the American Type Culture Collection (ATCC). Cos1 and HEK293 cells were cultured in DMEM with 10% FBS. Transfection of HEK293 and COS-1 cells were performed using X-tremeGENE™ HP DNA transfection reagent (#6366244001, Sigma-Aldrich, St. Louis, MO). The cAMPr lentivirus was packaged in HEK293T cells by co-transfection with pLVX-cAMPr-puro, ΔNRF and pCMV-VSV-G. The stable cAMPr-expressing HEK293 cells were established by lentiviral transduction, followed by puromycin selection.

Generation of ADCY3/ADCY6 double KO HEK293 cells. To generate ADCY3/ADCY6 double KO HEK293 with stable expression of cAMPr, the stable cAMPr-expressing HEK293 cells were sorted for GFP into single cells in a 96-well plate at 24 hours after transfection with the pCMV-AncBE4max, pLenti-ADCY3gRNA-Zeo and pLenti-ADCY6gRNA-Zeo. The gRNA sequences are listed in the Table 2. The expanded individual cell clones were screened by PCR and Sanger sequencing.

Histology analysis and immunofluorescence staining. The skeletal muscles were embedded in optimal cutting temperature (OCT) compound, flash frozen using isopentane chilled in liquid nitrogen and kept at −80° C. until used. Cryosections were prepared using a cryostat Leica CM3054. For hematoxylin and eosin (H&E), transversely oriented sections (10 μm) were cut at mid-point, stained and imaged using a Nikon Ti-E inverted fluorescence microscope equipped with a Lumenera Infinity Color CCD camera and a Nikon Super Fluor 20×0.75 NA objective lens (Nikon Inc., Melville, NY) as previously described.

For immunofluorescence staining, frozen tissue sections (10 μm) were fixed with 4% paraformaldehyde for 10 minutes at room temperature. After washing with PBS, the slides were blocked with 5% BSA/PBS for one hour. The slides were incubated with the indicated primary antibodies (Table 3) at 4° C. overnight. After extensive washing with PBS, the slides were incubated with the secondary antibodies (1:400) (Table 3) for one hour at room temperature. The slides were sealed with VECTASHIELD Antifade Mounting Medium with DAPI (Vector Laboratory, Burlingame, CA). The images were taken under a LSM780 microscope (Zeiss, Germany) and assembled to figures using Adobe Photoshop 21.0.2 (Adobe, San Jose, CA). Fiber size and CNF quantification were carried out using Myosight with manual calibration on the entire cross-sections of GA muscles.

In vivo protein synthesis measurements. In vivo protein synthesis was measured by using the SUNSET technique. Briefly, WT and BVES-KO mice with 3-month-old were anesthetized and then given an intraperitoneal (I.P.) injection of 0.040 μmol/g puromycin (P8833, Sigma-Aldrich, St. Louis, MO) dissolved in 100 μl of PBS. At 30 min after I.P. injection, muscles were collected and frozen in liquid nitrogen for Western blot analysis. A mouse IgG2a monoclonal anti-puromycin antibody (clone 12D10, 1:5000) was used to detect puromycin incorporation.

Intramuscular administration of AAV9-BVES into mice. AAV9 vectors were produced at Andelyn Biosciences (Columbus, OH) and titered by digital droplet PCR. AAV9-BVES viral particles (2×10¹¹ vg, 25 μl) were injected into the right gastrocnemius compartment of male BVES-KO mice at 4 months of age. At 1 and 3 months after AAV9 injection, force measurement (see below) was performed. Mice were sacrificed at 1 or 3 months after treatment. Gastrocnemius muscles were dissected out for morphometric analysis, immunofluorescence experiments and Western blot.

Bortezomib Treatment. Bortezomib was purchased from Selleck Chemicals (#S0130, Houston, TX). A stock solution (5 mg/ml) dissolved in the vehicle 2% DMSO, 30% PEG300 and ddH₂O were aliquoted and stored at 80° C. An equal volume of Bortezomib (0.8 mg/kg diluted with the vehicle) or vehicle was I.P. injected once bi-weekly into BVES-KO mice (N=6 per treatment) at 12 weeks age, respectively. After two months of treatment, force measurement and exercise evaluation were performed. Mice were sacrificed after 3-month treatment. QU and GA muscles were processed for histopathology, immunofluorescence and Western blot analyses.

Serum CK measurement. Blood samples collected from mice were transferred to MiniCollect tubes (Greiner Bio-one) and allowed to clot at room temperature for 30 minutes. Serum was prepared by centrifugation at 5000 rpm for 10 minutes and stored in −80° C. freezer for further use. Serum CK levels were measured using CREATINE KINASE-SL kit (326-10, Sekisui Diagnostics, Burlington, MA).

Autophagic flux assay. A stock solution of colchicine (4 mg/ml in sterile ddH₂O, C9754, Sigma-Aldrich) was prepared and stored at −20° C. until the day of treatment. Immediately prior to administration, the colchicine stock solution was diluted to 0.1 mg/mL in sterile ddH₂O water. An equal volume of colchicine (0.4 mg/kg) or vehicle was administered to mice (6 weeks old) daily via I.P. injection for 7 days. Muscle tissues were harvested from treated mice on the day after the final treatment.

Force measurement. The muscle contractility was measured using an in vivo muscle test system (Aurora Scientific Inc) as previously described. Briefly, mice were anesthetized with 2% (w/v) isoflurane and anesthesia was maintained by 2% isoflurane (w/v) during muscle contractility measurement. Maximum plantarflexion tetanic torque was measured during a train of supramaximal electric stimulations of the tibial nerve (pulse frequency 150 Hz, pulse duration 0.2 ms) using the DMA v5.501 (Aurora Scientific Inc).

Exercise and exhaustion assay. Time and distance to exhaustion was performed as previously described. Briefly, prior to training, the randomized mice were firstly acclimated to the treadmill (LE8710MTS, Harvard Apparatus) for two consecutive days at slow speed (10 cm/s) for 5 min. On the third day, mice were placed on an uphill) (15° treadmill with an initial speed of 10 cm/s, increased every 4 min by 5 cm/s. Mice were considered to be exhausted when the animal's hindlimbs remained on the electric grid for more than 10 s. Time and distance were automatically collected via the software SeDaCOM (Harvard Apparatus).

Drop-out assay. The drop-out assay was performed according to the previous study. Briefly, BVES-KO and age/sex-matched WT mice were initially trained (5 m/min running for 5 min each time, running for three times each day for three days) on treadmill. Then the mice were subjected to treadmill running at 10 m/min for 6 h. Twenty hours after the initial exercise training, mice were subjected to running at 6, 8, 10, 12, 14, and 16 m/min each for 3 min on the treadmill to test the capacity of recovery from muscle injury. The number of times the mice fail to run forward and touch the bottom of the electric grid of the treadmill and remain there for over 10 s was recorded as drop-out. Drop-outs of each mouse at each different speed were recorded.

Voluntary running wheel. In this study, BVES-KO and age/sex-matched WT mice were individually housed in cages equipped with voluntary free-spinning running wheels (0297-0521, Columbus Instruments, Columbus, OH) for 9 days. The voluntary running activity were recorded by wheel rotations at 2 h intervals.

Western blot. The cells and tissues were homogenized/lysed with cold RIPA buffer supplemented with protease inhibitors, and the extracted proteins were quantified by DC™ Protein Assay Reagent (Bio-Rad, Hercules, CA). The membrane extraction was performed using the Membrane Protein Extraction Kit (ab65400, Abcam, Waltham, MA) according to the manufacturer's instruction. The extracted protein samples were separated by stain-free SDS-PAGE gels (Bio-Rad, 4-15%) and transferred onto Nitrocellulose Membranes (0.45 μm). The membrane was incubated with the primary antibodies (Table 3) at 4° C. overnight. Secondary HRP-conjugated goat anti-mouse (1:4000) and goat anti-rabbit (1:4000) antibodies were obtained from Cell Signaling Technology (Danvers, MA). The membranes were developed using ECL Western blotting substrate (Pierce Biotechnology, Rockford, IL) and images were taken on ChemiDoc XRS+system (BioRad, Hercules, CA). Western blots were quantified using Image Lab 6.0.1 software (Bio-Rad Laboratories, Hercules, CA) according to the manufacturer's instruction.

RNA extraction and quantitative RT-PCR analysis. Total RNA was extracted from mouse tissues with Trizol. First-strand cDNA was synthesized using RevertAid RT Reverse Transcription Kit (K1691, ThermoFisher Scientific, Waltham, MA). Real-time PCR was performed using PerfeCTa SYBR Green FastMix (95074, QuantaBio, Beverly, MA) in QuantStudio™ 5 Real-Time PCR Systems (ThermoFisher Scientific). Samples were normalized for expression of Gapdh and analyzed by the 2-ΔΔCt method.

Immunoprecipitation and mass spectrum. The co-immunoprecipitation assay was performed as previously described from Cos-1 cells co-transfected with ADCY9-FLAG and BVES-3×HA. Gastrocnemius muscles from WT mice and BVES-KO mice with I.M. injection of AAV9-BVES-3×HA were lysed in the lysis buffer [25 mM Tris-HCl (pH 7.4), 150 mM NaCl, 5% Glycerol, 1% Triton X-100, 2 mM EDTA, and 1 mM DTT supplemented with protease inhibitor cocktail (Roche)]. Immunoprecipitation was performed by incubation with Pierce™ Anti-HA Magnetic Beads (#88836, ThermoFisher Scientific) for 1 h at 4° C. The beads were washed for four times with the lysis buffer. The immunoprecipitated samples were first examined by Western blot and then subjected for mass spectrometric analysis at the Ohio State University Comprehensive Cancer Center Proteomic Shared Resources.

FDB muscle electroporation and isolation. The plasmids RFP-LC3B or mCherry-GFP-LC3B were transfected into FDB muscles by electroporation as described previously. Briefly, the FDB muscle was injected with 10 μL of 2 mg/ml hyaluronidase solution (H4272, Sigma-Aldrich, St. Louis, MO). Two hours later, a total of 20-50 μg of the indicated plasmids was injected into the FDB muscles. After 15 min, the FDB muscles were electroporated using ECM630 (BTX, Holliston, MA) with the parameters (10 pulses, 20 ms in duration/each, 200 V). FDB muscle fibers were enzymatically isolated at 6 days after electroporation and placed in Tyrode's solution (119 mM NaCl, 5 mM KCl, 25 mM HEPES, 2 mM CaCl2, 2 mM MgCl2, 6 g/L glucose, pH 7.4) for live cell imaging, which was taken using Zeiss 780.

CAMPr reporter Assay. The ADCY3/ADCY6 double KO HEK293 cells with stable expression of cAMPr were seeded in a 35-mm glass-bottom dish coated with collagen (A1048301, ThermoFisher Scientific, Waltham, MA). The cells were transfected with the indicated vectors including pmCherry-C1, pCMV-ADCY9-Flag and pCMV-BVES-HA. At 24 h after transfection, the cells were washed twice with 1×Hanks' balanced salt solution and imaged under a Nikon Ti-E fluorescence microscope (Nikon, Melville, NY), equipped with a Zyla 5.5 sCMOS camera (Andor, Concord, MA) as well as NIS-Elements AR version 4.50 (Nikon, Melville, NY). Time series images were acquired every 4 s for 8 min. 500 μM IBMX was added to the culture dish at about 30 s after the fluorescence signal reaching a stable baseline. The CAMPr fluorescence signal data after background subtraction were plotted using Graphpad Prism 8.0.1.

CAMP ELISA Assay. The CAMP levels in skeletal muscles were measured using the cAMP Assay Kit (ab65355; Abcam, Waltham, MA) according to the manufacturer's instruction. Briefly, the 8-month-old WT and BVES-KO GA muscles were homogenized in 0.1 M HCl.

The amount of CAMP-HRP bound to the plate was determined by reading the colorimetric HRP activity at OD450 nm following sample acetylation. The measured CAMP values were normalized to the total protein content.

Statistical analysis. The data are expressed as mean±the standard error of the mean (S.E.M.). Statistical differences were determined by two-tailed unpaired Student's t test for two groups and one-way ANOVA with Turkey's post tests for multiple group comparisons using Prism 8.0.1 (Graphpad Software, La Jolla, California). A p value <0.05 was considered to be significant.

### Example 2. Systemic AAV9.BVES Delivery Ameliorates Muscular Dystrophy in a Mouse Model of LGMDR25

Limb-girdle muscular dystrophies (LGMDs) are a highly heterogeneous group of muscular disorders that are characterized by progressive muscle weakness and wasting in legs and arms with some affected by heart dysfunction. Genetic defects in blood vessel epicardial substance (BVES) gene (also known as Popeye domain containing protein 1 or POPDC1) were recently identified to cause autosomal recessive limb-girdle muscular dystrophy type X or R25 (LGMD2X or LGMDR25). LGMDR25 patients showed elevated serum creatine kinase (CK) and progressive proximal limb muscle weakness and atrophy with many eventually becoming wheelchair bounded. Heart rhythm abnormalities including arrhythmia, atrioventricular (AV) block and sinus bradycardia have also been recorded in LGMDR25 patients. The precise prevalence of LGMDR25 is unknown but an increasing number of patients carrying BVES mutations have recently been identified. To date, there are no treatments available for LGMDR25 patients.

BVES encodes a cAMP-binding transmembrane protein, belonging to the Popeye domain containing (POPDC) protein family, which contains two other members POPDC2 and POPDC3. All POPDC proteins are highly expressed in striated muscles. Interestingly, patients carrying mutations in BVES often display a loss of membrane localization of POPDC2, a risk protein of cardiac arrhythmia. BVES has been implicated in various cellular processes, including protein trafficking, myoblast differentiation, cell migration, cell proliferation and signaling cascades. Consistent with patient studies, BVES-deficient zebrafish displayed muscular dystrophy as well as AV block in heart, and BVES ablated mice displayed the defect of muscle regeneration upon cardiotoxin (CTX) challenge in skeletal muscle and stress-induced bradycardia. A new BVES knockout (BVES-KO) mouse model was recently established, which developed muscular dystrophy and atrophy and faithfully recapitulated the pathological features of LGMDR25 patients, supporting its use for preclinical translational studies.

AAV-mediated gene therapy has been widely exploited for various genetic diseases including muscular dystrophies, due to the broad tissue tropism and safety in human. In particular, AAV gene therapy has recently received Food and Drug Administration (FDA) approval for hereditary retinal dystrophy (Luxturna) and spinal muscular atrophy (Zolgensma). AAV9 has been shown to be highly efficient at transducing major tissues including the liver, heart, skeletal muscle, and central nervous system. Here, this study exploited the feasibility to treat LGMDR25 by AAV9-mediated systemic delivery of a BVES transgene using the BVES-KO mice. These results showed that both neonatal and adult administration of AAV9.BVES can dramatically improve the function and pathology of BVES-KO mice, without notable serious adverse events. This study highlights the great promise of AAV9.BVES gene therapy for LGMDR25.

### AAV9.BVES Transduces Skeletal Muscle and Heart Efficiently in BVES-KO Mice

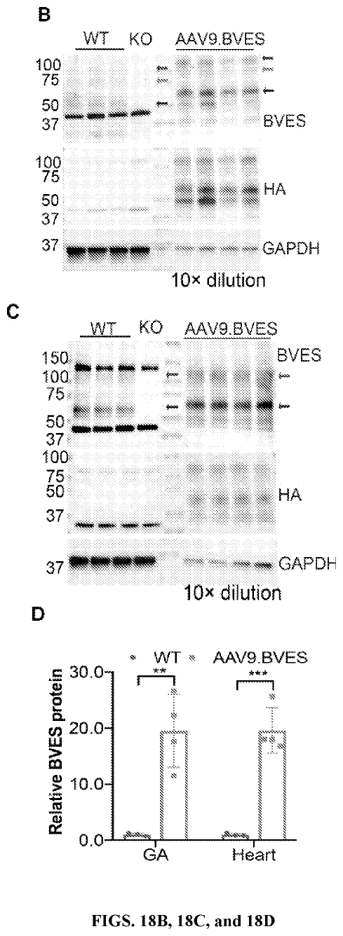

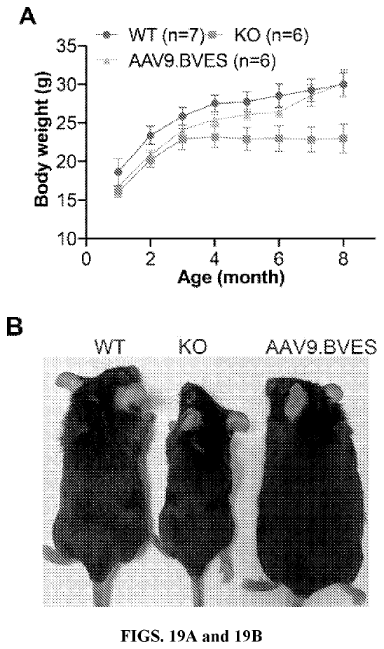

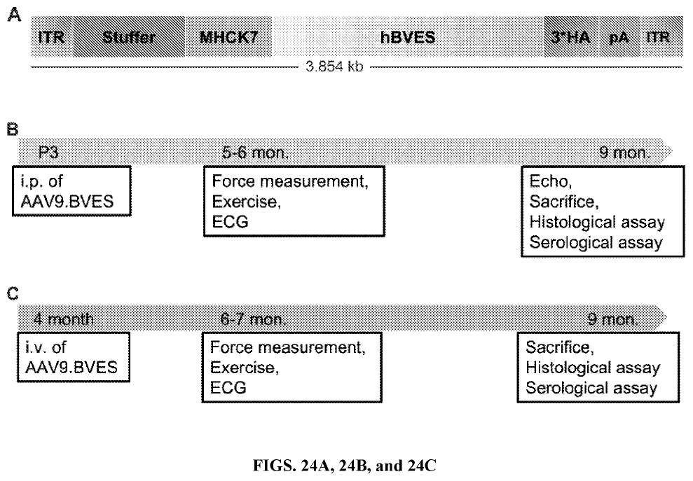

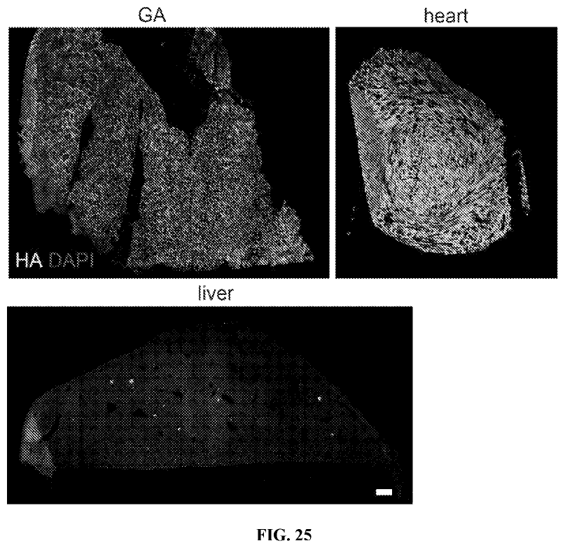

To generate AAV9.BVES, this study constructed an AAV transfer plasmid by placing the human BVES cDNA fused with 3×HA tag under the control of a skeletal and cardiac muscle-specific promoter MHCK7 (FIG. 24A). To test if early intervention with AAV9.BVES can prevent the disease development in BVES-KO mice, AAV9.BVES (1×10¹⁴ vg/kg) was administered into BVES-KO pups (22 male and 8 female) at postnatal day 3 (P3) through intraperitoneal (ip) injection (FIG. 24B). One injected mouse was sacrificed at 3 months of age to examine the expression of the BVES transgene. As shown in FIG. 18A and FIG. 25, BVES was mainly expressed at the sarcolemma of various skeletal muscle fibers including gastrocnemius (GA), quadriceps (QU), tibial anterior (TA), diaphragm (DIA) and soleus (SOL). Some intercellular staining was also observed, particularly in cardiomyocytes (FIGS. 18A and 25). BVES was hardly detectable in the other major organs such as liver, lung, kidney and colon, indicating that the combination of AAV9 delivery and the muscle-specific MHCK7 promoter is highly efficient in restricting the expression of BVES transgene in striated muscle fibers.

Western blot analysis further confirmed the transgene expression in skeletal muscle (FIG. 18B) and heart (FIG. 18C) of BVES-KO mice treated with AAV9.BVES when sacrificed at 9 months of age. Endogenous BVES was detected as three major bands in skeletal muscle and two major bands in heart samples, consistent with previous reports that BVES can exist as monomer, dimer and glycosylated forms. The BVES antibody also detected some non-specific bands as they appeared in both WT and BVES-KO samples. The BVES transgene was strongly expressed in both the skeletal muscle and heart of BVES-KO mice treated with AAV9.BVES as detected by the antibodies against BVES or the HA tag (FIGS. 18B and 18C). Densitometry analysis showed that the expression of the BVES transgene was nearly 20 folds of WT levels in both GA and heart muscles of BVES-KO mice treated with AAV9.BVES (FIG. 18D).

### Neonatal Delivery of AAV9.BVES Normalizes Muscle Function and Exercise Performance in BVES-KO Mice

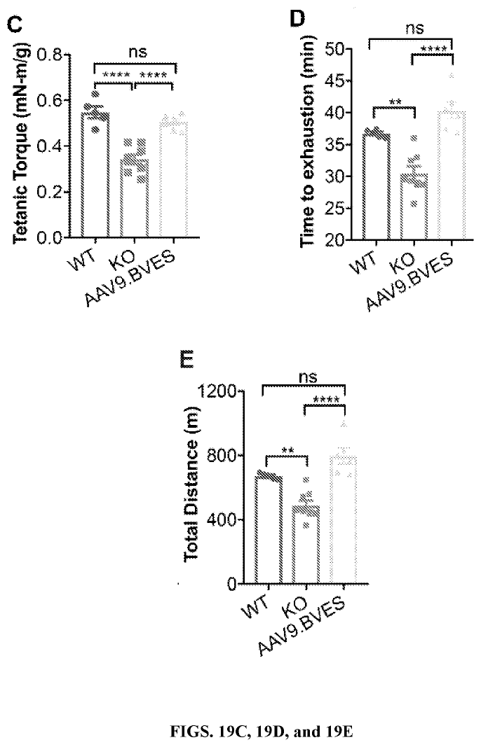

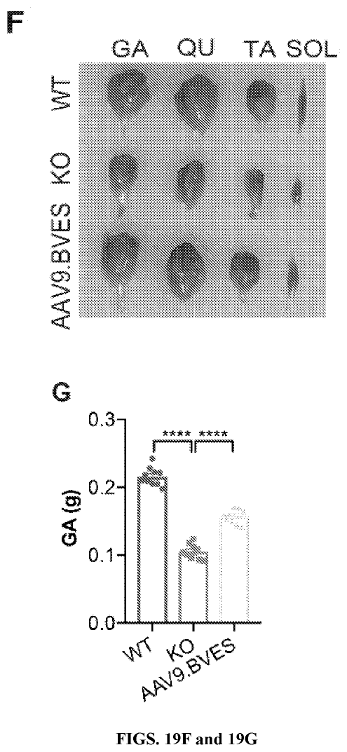

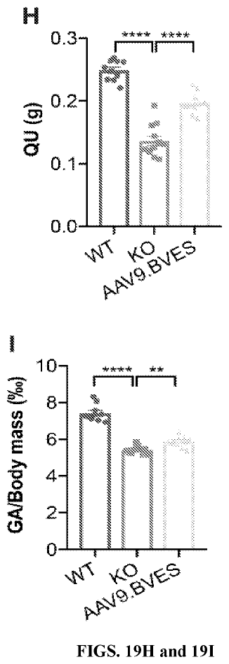

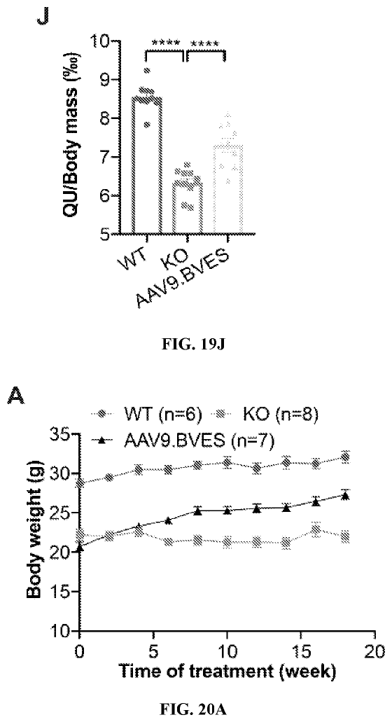

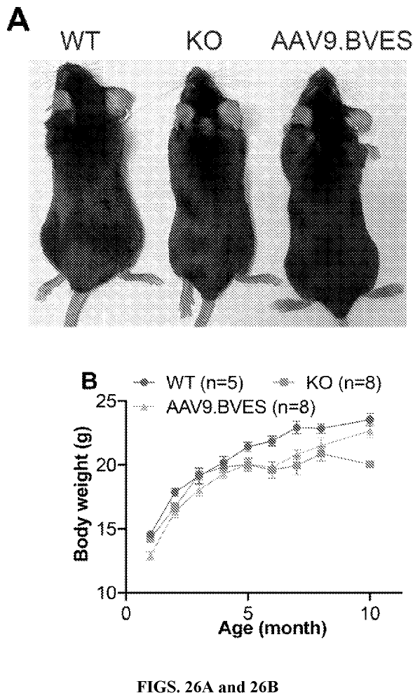

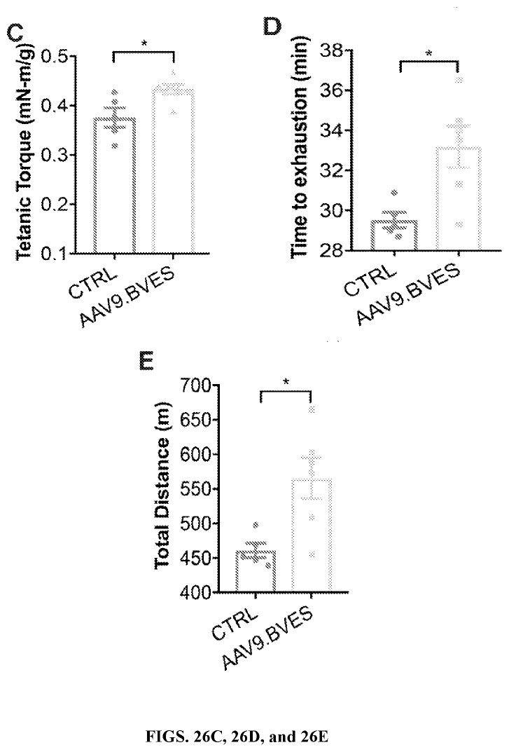

To examine whether neonatal administration of AAV9.BVES can prevent the disease development, the BVES-KO mice treated with or without AAV9.BVES at P3 were monitored monthly for body weight gain. As compared to age-matched WT males, the male BVES-KO mice showed significantly reduced body weight gain, particularly after 3 months of age (FIG. 19A). AAV9.BVES greatly improved the body weight gain in BVES-KO mice, and there was no significant difference in the body weight between the treated BVES-KO and WT mice at 8 months of age (30.0±1.5 g vs 30.1±1.8 g, p=1.0). When sacrificed at 9 months of age, the AAV9.BVES-treated BVES-KO mice were not visually different from the WT mice while the untreated BVES-KO mice looked much smaller than the WT controls (FIG. 19B). Similarly, AAV9.BVES treated female BVES-KO mice also showed significant improvement in body weight gain, similar to their WT counterparts (FIGS. 26A and 26B).

To test if AAV9.BVES treatment improves muscle function, this study first measured the muscle contractility using an in vivo muscle test system. Maximum plantarflexion tetanic torque was measured during supramaximal electric stimulation of the tibial nerve at 150 Hz. As compared to age-matched WT mice, the BVES-KO mice at 5 months of age produced significantly reduced torque while AAV9.BVES treatment fully normalized the contractile strength in the KO mice (FIG. 19C). Similar improvement in the contractile strength was also detected in AAV9.BVES treated female KO mice (FIG. 26C). Next, the mice were subjected to uphill) (15° treadmill running till exhaustion. Both the time to exhaustion (FIG. 19D) and the total running distance (FIG. 19E) were significantly decreased in male BVES-KO mice as compared to WT, however, AAV9.BVES treatment fully normalized both parameters in male BVES-KO mice. Similarly, AAV9.BVES significantly improved exercise performance in female BVES-KO mice (FIGS. 26D and 26E). These studies showed that neonatal administration of AAV9.BVES completely restored the body weight gain, contractile strength, and exercise performance in BVES-KO mice. Consistent with the reduced body weight, this study found that the muscle mass was dramatically reduced in GA and QU muscles of BVES-KO mice, which was significantly improved by AAV9.BVES treatment (FIG. 19F-19J).

### Adult Administration of AAV9.BVES Also Significantly Improves Muscle Function and Exercise Performance in BVES-KO Mice

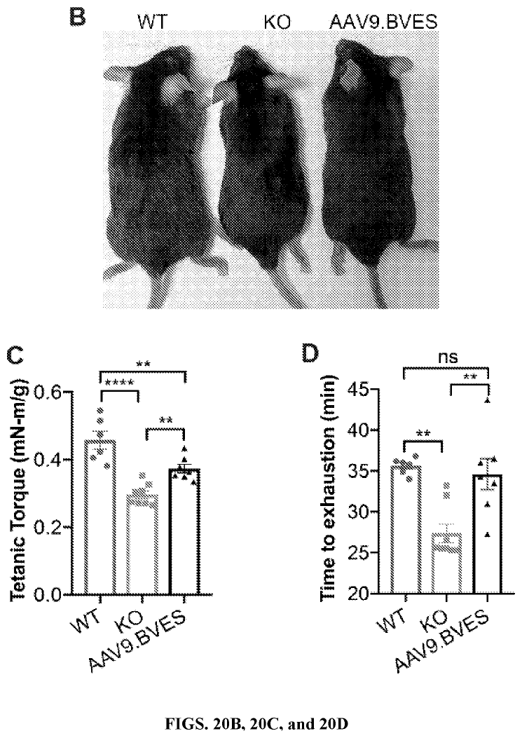

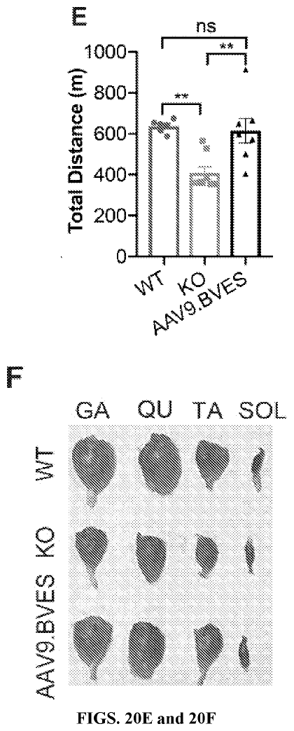

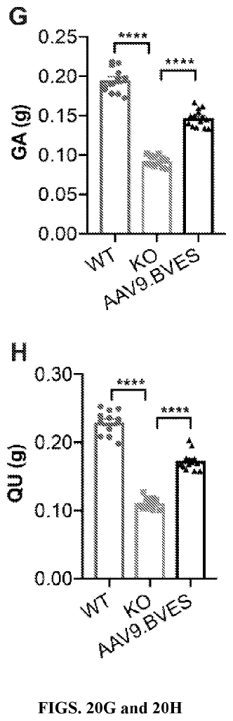

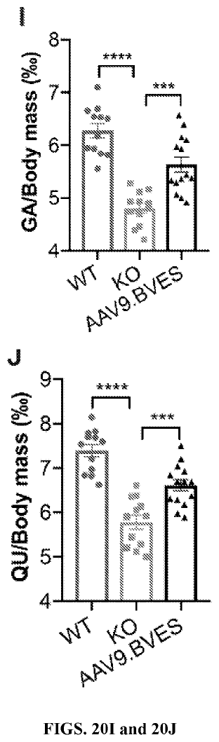

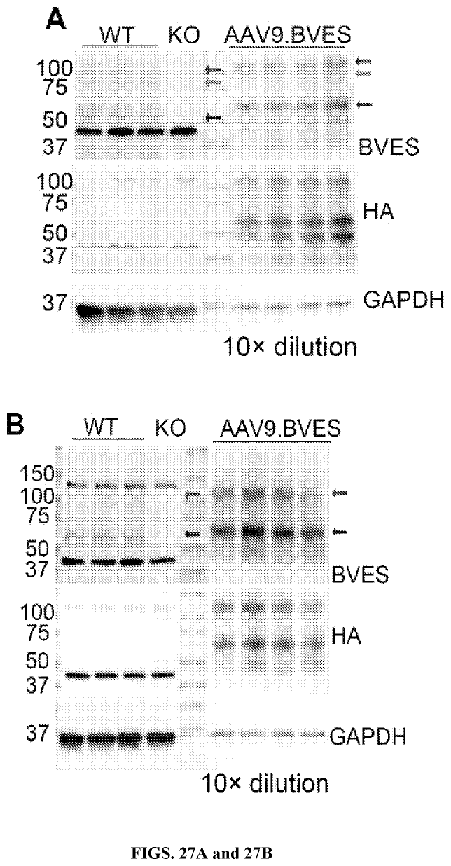

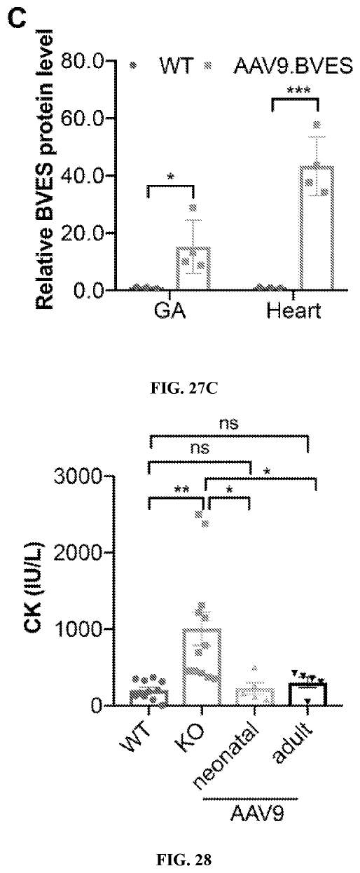

To further study the translational of AAV9.BVES gene therapy, this study examined whether systemic administration of AAV9.BVES into adult BVES-KO mice after disease onset (FIG. 24C) can show beneficial effects on muscle function and exercise performance. As this study revealed that BVES-KO mice displayed the retarded body weight gain starting at around 3 months of age (see FIG. 19), the 4-month-old BVES-KO mice were treated with AAV9.BVES through tail vein injection and measured the body weight, force contractility, exercise performance and muscle mass as described above. Western blot analysis confirmed the transgene expression in skeletal muscle and heart of BVES-KO mice treated with AAV9.BVES when sacrificed at 9 months of age (FIG. 27A-27C). While the untreated BVES-KO mice failed to gain weight, AAV9.BVES treated BVES-KO animals showed a steady increase in their body weight within 18 weeks after treatment, although still lighter than their age- and gender-matched WT controls (FIG. 20A). At 6 weeks after AAV9.BVES treatment, significant differences were observed in the body weight between treated and untreated BVES-KO mice (FIG. 20A). The AAV9.BVES-treated BVES-KO mice were visually larger than the untreated BVES-KO animals (FIG. 20B). At two months after AAV9.BVES treatment, the contractile strength was significantly increased as compared to the untreated controls (FIG. 20C). Moreover, AAV9.BVES treatment in BVES-KO mice restored their exercise performance to the WT levels (FIGS. 20D and 20E). At 9 months of age, the mice were sacrificed to study the impact of AAV9.BVES treatment on muscle mass. As shown in FIG. 20F-20J, AAV9.BVES treatment significantly increased the mass of GA and QU muscles.

Together, these results showed that AAV9.BVES gene transfer into adult BVES-KO mice can also significantly improve muscle function, exercise performance and muscle mass even after the disease onset.

### AAV9.BVES Gene Therapy Improves Muscle Pathology

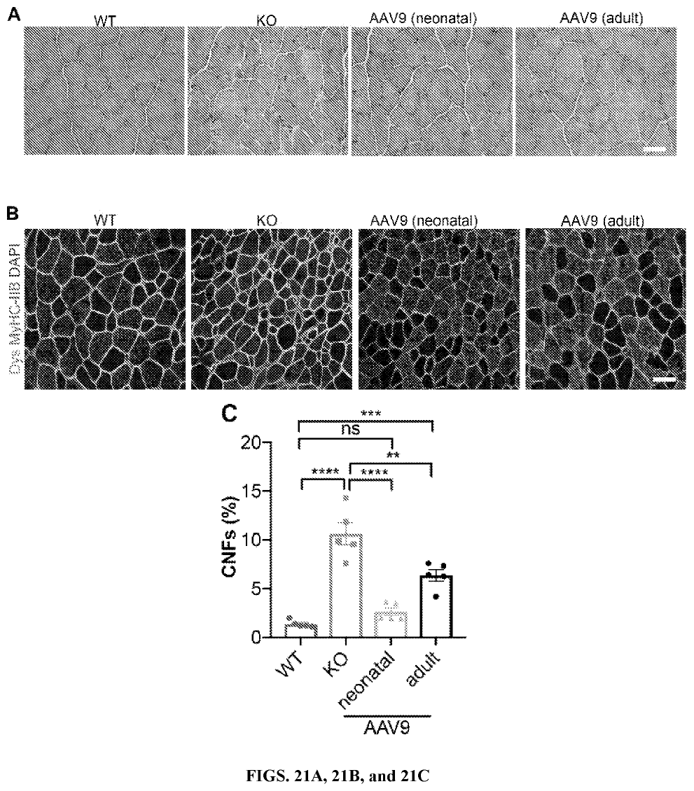

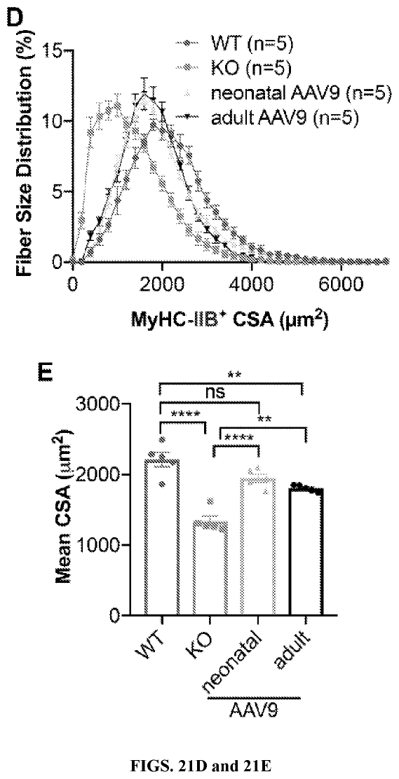

Like the patients with LGMDR25, BVES-KO mice exhibited a phenotype of muscular dystrophy and atrophy as evidenced by elevated serum CK levels, muscle necrosis, significant fiber size variability, centrally nucleated muscle fibers (CNFs) and smaller muscle mass. Either adult or neonatal administration of single-dose AAV9.BVES significantly normalized the serum CK levels (FIG. 28). H&E staining showed that a considerable number of CNFs and necrotic fibers were present in BVES-KO muscles and that systemic AAV9.BVES administration at either neonatal or adult stage ameliorated these pathological alterations in the BVES-KO mice (FIG. 21A). To quantify the muscle size and CNF percentages, immunofluorescence staining was performed with antibodies against dystrophin, myosin heavy chain 2B and DAPI (FIG. 21B), as a recent study showed that the type IIB muscle fibers were most affected in BVES-KO mice (H. Li et al., manuscript submitted). With the expected low percentage of CNFs in the GA muscles of WT mice, an average of 10.6±1.1% CNFs was observed in male KO mice at 9 months of age (FIG. 21C). Either neonatal or adult administration of AAV9.BVES significantly reduced the percentage of CNFs (2.6±0.4% and 6.4±0.6%, respectively) (FIG. 21C) and increased the percentage of muscle fibers with larger area (FIGS. 21D and 21E), with neonatal injection showing higher effects.

### AAV9.BVES Gene Transfer Improves Heart Rhythm.

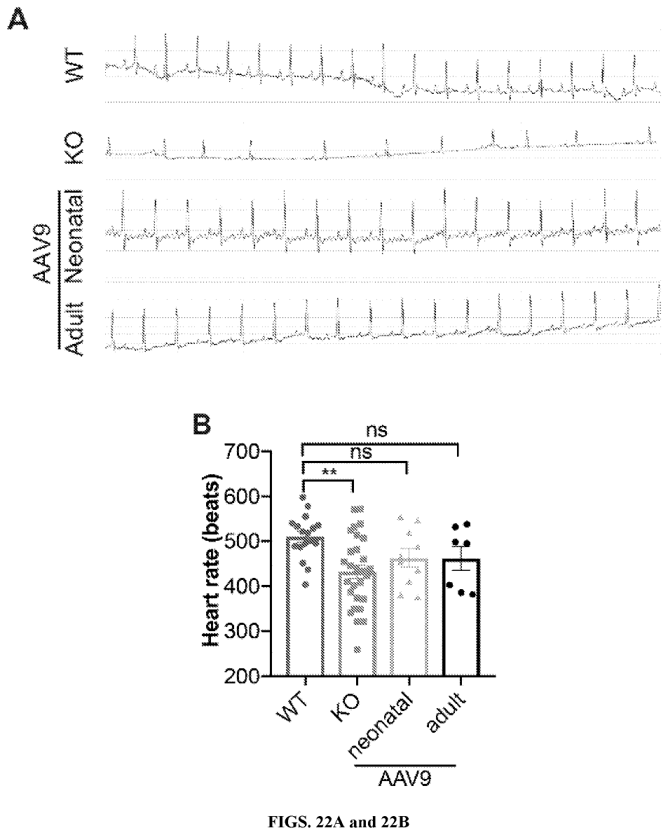

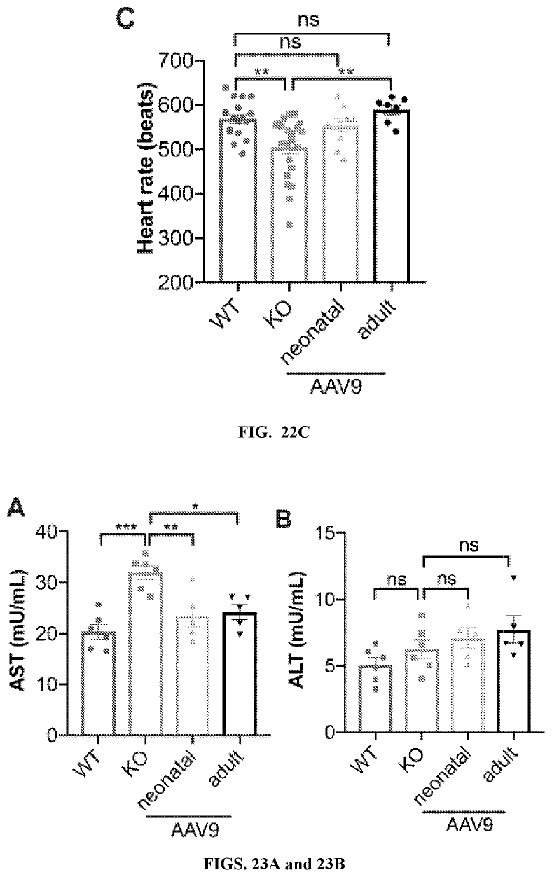

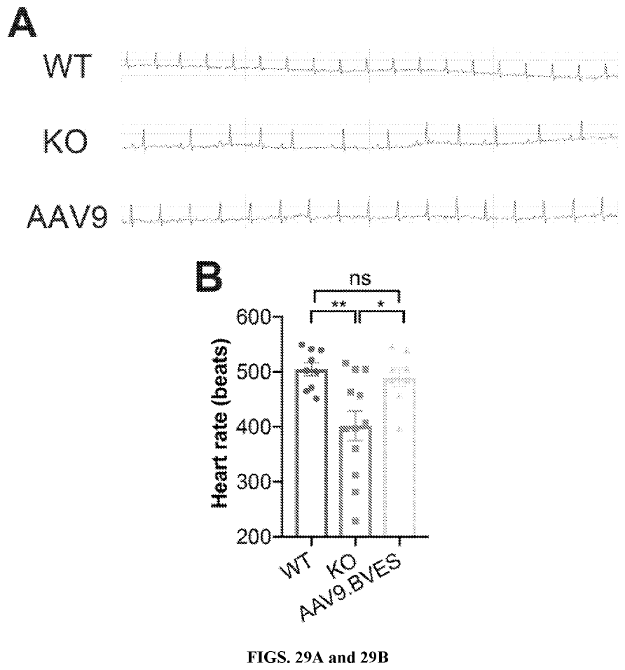

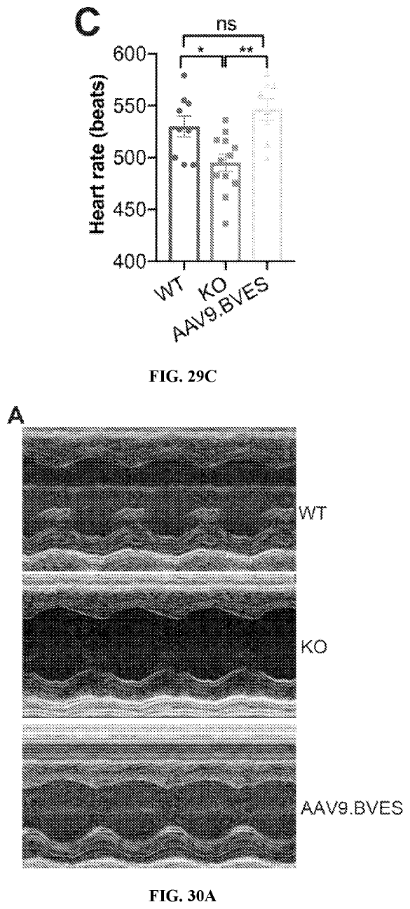

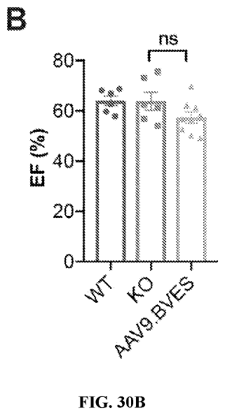

Previous studies showed that patients with LGMDR25 presented arrythmia, AV block and sinus bradycardia. Similarly, the BVES-KO mice displayed lower heart rate and severe repetitive drops of the heart rate during and after swimming stress. To examine whether AAV9.BVES gene transfer can correct the heart rate defect, this study monitored male and female mice by electrocardiogram (ECG). At the rest condition, seven out of 21 untreated male BVES-KO mice showed the delayed or skipped beats. AAV9.BVES normalized the ECG irregularity in BVES-KO mice treated at either neonatal or adult stage (FIG. 22A). AAV9.BVES showed similar benefits for improving ECG in female BVES-KO mice where abnormal heart beats were observed in nine out of 15 mice (FIG. 29A). Under the rest condition, the average heart rate of untreated male BVES-KO mice was significantly lower than that of WT (FIGS. 22B and 22D). The animals were then subjected to an episode of 10-min uphill treadmill running, immediately followed by ECG recording. The mice in all groups showed an increase in heart rate following the treadmill running with the BVES-KO mice still displaying reduced heart rate as compared to the WT animals (FIG. 22B-22E). AAV9.BVES treatment at either neonatal or adult stage normalized the heart rate of BVES-KO mice in response to exercise-induced stress (FIGS. 22C and 22E). Similar effects were observed in female animals (FIGS. 29B and 29C). Moreover, this study measured the cardiac function by echocardiography. No significant differences were found in ejection fraction among the animals at 9 months of age, regardless of genotype or treatment (FIGS. 30A and 30B).

### AAV9.BVES has Minimal Toxicity in BVES-KO Mice

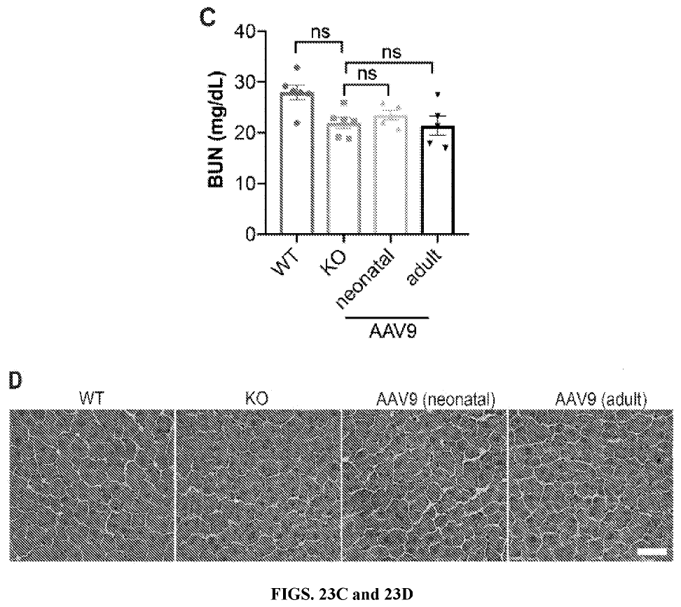

To examine the safety profile of AAV9.BVES, this study measured the serum aspartate aminotransferase (AST) and alanine aminotransferase (ALT) for liver toxicity, blood urine nitrogen (BUN) for kidney toxicity. The BVES-KO mice showed elevated AST (FIG. 23B) and normal ALT (FIG. 23C) levels as compared to the WT animals, which were not unexpected as increases in these enzymes were frequently observed in muscular dystrophy patients and animals¹⁹. Consistent with the observation that AAV.BVES treatment improved the muscular dystrophy phenotype in BVES-KO mice, AAV9.BVES treatment also normalized the serum levels of AST (FIGS. 23B and 23C). Measurement of ALT and BUN did not show significant changes in the treated or untreated BVES-KO mice (FIG. 23D). H&E staining showed no detectable alterations in livers of BVES-KO mice treated with or without AAV9.BVES (FIG. 23E). These results indicate that systemic delivery of AAV9.BVES did not induce obvious liver or kidney toxicity in BVES-KO mice.

This study evaluated the efficacy and safety of recombinant AAV carrying the human BVES transgene as a promising gene therapy for LGMDR25. The AAV9 serotype and muscle-specific promoter were used to better target skeletal and cardiac muscles. The data here showed that systemic administration of a single dose of AAV9.BVES before or after the disease onset ameliorated the muscular dystrophy and heart rhythm abnormality without overt adverse effects in BVES-KO mice. These findings provided a foundation for translating AAV-mediated BVES gene transfer to LGMDR25 patients.

A recent study characterizing the phenotype of BVES-KO mice showed that this mouse model accurately recapitulates the clinical pathological features of LGMDR25, supporting its use for preclinical translational studies. Like LGMDR25 patients, the BVES-KO mice developed muscular dystrophy, muscular atrophy, and cardiac conduction defect. Thus, targeting both the skeletal and cardiac muscles must be considered for the long-term treatment of this disease. The use of AAV9 and the skeletal and cardiac muscle-specific promoter MHCK7 allows for enhanced transgene expression in skeletal and cardiac muscles with little off-target tissue expression following systemic delivery. This study demonstrated that systemic delivery of AAV9.BVES leads to nearly complete transduction and restoration of BVES expression in various limb skeletal muscles, diaphragm and cardiac muscle with little detectable expression in liver, lung, kidney or colon. This was accompanied by a remarkable improvement in the body weight gain, muscle mass, muscle contractility, exercise performance, muscle histopathology and ECG regularity. Of note, the neonatal intervention with a lower dosage appeared to be more effective, as evidenced by higher body weight, lower CNFs, better force production and exercise capacity, when compared to the treatment at a later stage of the disease in BVES-KO mice. Similar observations were found in AAV9-FKRP gene therapy for LGMD2I.

A high transgene expression was observed with the MHCK7 promoter. The expression of BVES protein following AAV9.BVES delivery was estimated to be around 20 folds of WT levels, consistent with previous reports showing the strong activity of the MHCK7 promoter. This high level of BVES expression did not show any evidence of toxicity during the nine months of study. This contrasts with previous studies of some other LGMD genes such as dysferlin, calpain3 and caveolin-3, for which the overexpression can cause overt pathology in skeletal muscle and/or heart. Moreover, recently engineered AAV capsids with improved muscle and heart transduction efficiency such as the AAVMYO and MyoAAV can further lower the required dose and thus improve the safety profile for BVES gene therapy.

Gene replacement therapy has been increasingly explored to treat various forms of muscular dystrophies including Duchenne muscular dystrophy (DMD) and LGMDs. Several preclinical studies showed a very promising perspective, such as rAAV6-microdystrophin for DMD, rAAVrh74.MHCK7.hSGCA for LGMD2D, scAAV.MHCK7.hSGCB for LGMD2E, AAV8-desm-hSGCG for LGMD2C and AAV9-FKRP for LGMD2I, and some of these have moved into clinical trials including SGT-001 (AAV-microdystrophin), SRP-9003 (rAAVrh74.MHCK7.hSGCB) and SRP9004 (rAAVrh74.MHCK7.hSGCA) (clinicaltrials.gov/). This study places LGMDR25 onto the growing list of AAV gene therapy. So far eight pathogenic mutations in BVES have been reported in LGMDR25 patients, including S201F, R88Ter, Del56 V217-K272, R143Ter, S263Ter, M1G, Q153Ter, and I193S. In addition, the decreased expression of BVES caused by R129W mutation in human has been linked to the Sporadic Tetralogy of Fallot, the most common type of congenital heart disease. The AAV9.BVES gene therapy offers a mutation-independent approach for the treatment of LGMDR25.

### Materials and Methods

Mice. All animal studies were reviewed and approved by the Institutional Animal Care and Use Committee (IACUC) of The Ohio State University. C57BL/6N mice were purchased from The Jackson Laboratory (Bar Harbor, ME, USA). The BVES-KO mice (C57BL/6N-Bvestm1.1 (KOMP) Vlcg/MbpMmucd) were obtained from Mutant Mouse Resource & Research Centers, UC Davis and maintained in a barrier facility. In BVES-KO mice, all coding exons were deleted via the Cre-mediated recombination. Identification of the mutant mice was performed by PCR genotyping of genomic DNA prepared from ear clips with the following primers: KO-F, 5′-ACTTGCTTTAAAAAACCTCCCACA ID (SEQ NO: 9), KO-R, 5′-AGTCACTAGCAAGAGATCTGCACCC (SEQ ID NO: 10); WT-F, 5′-AAGTGCTGGGATTAAAGGTGTGTGC (SEQ ID NO: 11), WT-R, 5′-AAGGACACATCACAGCTTCAGG (SEQ ID NO: 12). The WT and KO allele would produce a 164-bp and 771-bp band, respectively.

Systemic administration of AAV9.BVES in BVES-KO mice. AAV9.BVES viral particles were produced, purified and tittered at Andelyn Biosciences (Columbus, OH) as previously described. Titers are expressed as DNase resistant particles per ml (DRP/ml) and AAV9 titers used for the in vivo studies were 4×10¹³ DRP/ml. The male or female BVES-KO mice at postnatal day 3 were administered with AAV9.BVES viral particles (˜1×10¹⁴ vg/kg) via i.p. injection. For adult administration, the male BVES-KO mice at 4 months of age were injected with AAV9.BVES viral particles (2×10¹⁴ vg/kg) via tail vein.

Treadmill running. Total time and distance of running to exhaustion were performed as previously described. Briefly, the randomized mice were firstly acclimated to the treadmill (LE8710MTS, Harvard Apparatus) for two consecutive days at low speed (10 cm/s) for 5 min. At the third day, mice were placed on the uphill) (15° treadmill with the parameters: initial speed 10 cm/s, increased every 4 min by 5 cm/s. Mice were considered to be exhausted when the animal's hindlimbs remained on the electric grid for more than 10 s. Time and distance were automatically collected via the software SeDaCOM (Harvard Apparatus).

Force measurement. The muscle contractility was measured as previously described using an in vivo muscle test system equipped with software DMC LabBook (Aurora Scientific Inc). Mice were anesthetized with 3% (w/v) isoflurane and anesthesia was maintained by 2% isoflurane (w/v) during muscle contractility measurement. The tetanic torques from the posterior compartment muscles (including gastrocnemius, soleus and plantaris muscles) of mice were measured by electric stimulation of the sciatic nerve.

Electrocardiogram (ECG) recording. The ECG was measured using IX-BIO4 (iWorx Systems). C57BL/6J and BVES-KO mice with or without AAV9.BVES were anesthetized with 2% (w/v) isoflurane, and ECG was recorded for about 50 s under anesthesia before and immediately after 10-min treadmill running (5° uphill). The data were analyzed using the software Labscribe (iWorx Systems).

Echocardiography recording. C57BL/6J and BVES-KO mice with or without AAV9.BVES were anesthetized with 1-2% isoflurane, and echocardiography was measured using Vevo 2100 Ultrasound system. Echocardiography data were analyzed using VevoLab software to determine the left ventricle ejection fraction.

Immunofluorescence staining. Cryosections (10 μm) of mouse tissues were prepared using Cryostat 1905 (Leica Camera, Germany). The slides were fixed with 4% paraformaldehyde for 10 min at room temperature. After washing with PBS three times (5 min each), the slides were blocked with 5% BSA plus 5% normal serum for 1 h. The slides were then incubated with primary antibodies against HA (1:1000, #3724, Cell Signaling Technology, Danvers, MA), Dystrophin (E2660, 1:500, Spring Bioscience, Pleasanton, CA) or MyHC-IIB (1:50, 10F5, Developmental Studies Hybridoma Bank) at 4° C. overnight. Following extensive wash with PBS, the slides were incubated with the corresponding secondary antibodies Alexa Fluor 488 goat anti-rabbit IgG (A-11034, 1:400, Invitrogen, Carlsbad, CA) or Alexa Fluor 594 goat anti-mouse IgM (A-21044, 1:400, Invitrogen, Carlsbad, CA) for 1 h at room temperature. Finally, the slides were sealed with VECTASHIELD Antifade Mounting Medium with DAPI (Vector Laboratory, Burlingame, CA). All images were taken with a Zeiss 780 confocal microscope. Fiber size and CNF quantification were carried out using Myosight with manual calibration on the entire cross-sections of GA muscles.

Hematoxylin and eosin (H&E) staining. Ten μm frozen muscle and liver sections were fixed in 10% formaldehyde for 5 min at room temperature and then proceeded to the standard protocol of H&E staining. All images were taken under a Nikon Ti-E fluorescence microscope, magnification 200×(Nikon, Melville, NY).

Western blot. The mouse tissues were lysed with cold RIPA buffer supplemented with protease inhibitors (S8830, Sigma-Aldrich) as well as PhosSTOP (PHOSS-RO, Roche), and the extracted proteins were quantified by DC™ Protein Assay Reagent (BioRad). The extracted protein samples were separated by stain-free SDS-PAGE gels (4-15%, BioRad) and transferred onto nitrocellulose membranes (0.45 μm). Primary antibodies used include the rabbit polyclonal anti-BVES (1:1000, HPA014788, Sigma-Aldrich, St. Louis, MO), anti-HA (1:1000, #3724, Cell Signaling Technology, Danvers, MA) and anti-GAPDH (1:2000, MAB374, Cell Signaling Technology, Danvers, MA). Secondary HRP-conjugated goat anti-mouse (1:4000) and goat anti-rabbit (1:4000) antibodies were obtained from Cell Signaling Technology. The membranes were developed using ECL Western blot substrate (Pierce Biotechnology, Rockford, IL) and scanned by ChemiDoc XRS+system (BioRad, Hercules, CA). Western blots were quantified using ImageJ.

Serological analysis. Serum samples collected from the mice were stored at −80° C. for the biochemical assays. Measurements of CK (326-10, Sekisui Diagnostics), AST (ab105135, Abcam), ALT (ab105134, Abcam) and BUN (EIABUN, Thermo fisher) were performed according to the manufacturer's protocols.

Data analysis and statistics. The data were expressed as mean±the standard error of the mean (SEM), analyzed with GraphPad Prism 8.0.1 software (San Diego, California, USA) and final figures were assembled with Adobe Photoshop 2020. Statistical differences were determined by two-tailed unpaired Student's t test for two groups and one-way ANOVA with Turkey's post tests for multiple group comparisons using Prism 8.0.1 with the assumption of Gaussian distribution of residuals. A p<0.05 was considered to be significant.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1

| | Accession | Alternate | Molecular | | | AAV9- |
|---|---|---|---|---|---|---|
| Identified Proteins (1020) | Number | ID + Sheet1! | Weight | | CTRL | BVES-HA |
| Ankyrin-3 OS = Mus musculus | G5E8K5 | Ank3 | 214 | kDa | 0 | 242 |
| OX = 10090 GN = Ank3 PE = 1 SV = 1 | | | | | | |
| Keratin, type II cytoskeletal 7 | Q9DCV7 | Krt7 | 51 | kDa | 0 | 17 |
| OS = Mus musculus OX = 10090 | | | | | | |
| GN = Krt7 PE = 1 SV = 1 | | | | | | |
| Blood vessel epicardial substance | Q9ES83 | Bves | 41 | kDa | 0 | 40 |
| OS = Mus musculus OX = 10090 | | | | | | |
| GN = Bves PE = 1 SV = 1 | | | | | | |
| Ankyrin-2 OS = Mus musculus | Q8C8R3 | Ank2 | 426 | kDa | 0 | 36 |
| OX = 10090 GN = Ank2 PE = 1 SV = 2 | | | | | | |
| Plasma membrane calcium- | G5E829 | Atp2b1 | 135 | kDa | 0 | 34 |
| transporting ATPase 1 OS = Mus | | | | | | |
| musculus OX = 10090 GN = Atp2b1 | | | | | | |
| PE = 1 SV = 1 | | | | | | |
| Ankyrin-1 OS = Mus musculus | Q02357 | Ank1 | 204 | kDa | 0 | 31 |
| OX = 10090 GN = Ank1 PE = 1 SV = 2 | | | | | | |
| Popeye domain-containing protein | Q9ES82 | Popdc2 | 41 | kDa | 0 | 29 |
| 2 OS = Mus musculus OX = 10090 | | | | | | |
| GN = Popdc2 PE = 1 SV = 1 | | | | | | |
| Spectrin alpha chain, erythrocytic 1 | P08032 | Spta1 | 280 | kDa | 0 | 27 |
| OS = Mus musculus OX = 10090 | | | | | | |
| GN = Spta1 PE = 1 SV = 3 | | | | | | |
| Tubulin alpha-1B chain OS = Mus | P05213 | Tuba1b | 50 | kDa | 0 | 13 |
| musculus OX = 10090 GN = Tuba1b | (+1) | | | | | |
| PE = 1 SV = 2 | | | | | | |
| Sodium channel protein type 4 | Q9ER60 | Scn4a | 209 | kDa | 0 | 19 |
| subunit alpha OS = Mus musculus | | | | | | |
| OX = 10090 GN = Scn4a PE = 1 SV = 1 | | | | | | |
| Sodium/potassium-transporting | Q8VDN2 | Atp1a1 | 113 | kDa | 0 | 12 |
| ATPase subunit alpha-1 OS = Mus | | | | | | |
| musculus OX = 10090 GN = Atp1a1 | | | | | | |
| PE = 1 SV = 1 | | | | | | |
| ATP-binding cassette sub-family C | P70170 | Abcc9 | 174 | kDa | 0 | 14 |
| member 9 OS = Mus musculus | | | | | | |
| OX = 10090 GN = Abcc9 PE = 1 | | | | | | |
| SV = 2 | | | | | | |
| SUN domain-containing protein 2 | Q8BJS4 | Sun2 | 82 | kDa | 0 | 14 |
| OS = Mus musculus OX = 10090 | | | | | | |
| GN = Sun2 PE = 1 SV = 3 | | | | | | |
| Voltage-dependent L-type calcium | Q02789 | Cacna1s | 213 | kDa | 0 | 13 |
| channel subunit alpha-1s OS = Mus | | | | | | |
| musculus OX = 10090 GN = Cacna1s | | | | | | |
| PE = 1 SV = 2 | | | | | | |
| Chloride channel protein 1 | Q64347 | Clcn1 | 110 | kDa | 0 | 13 |
| OS = Mus musculus OX = 10090 | | | | | | |
| GN = Clcn1 PE = 1 SV = 3 | | | | | | |
| Calcium/calmodulin-dependent | Q923T9 | Camk2g | 60 | kDa | 0 | 13 |
| protein kinase type II subunit | | | | | | |
| gamma OS = Mus musculus | | | | | | |
| OX = 10090 GN = Camk2g PE = 1 | | | | | | |
| SV = 1 | | | | | | |
| Ribosomal protein S6 kinase alpha- | Q9WUT3 | Rps6ka2 | 83 | kDa | 0 | 12 |
| 2 OS = Mus musculus OX = 10090 | | | | | | |
| GN = Rps6ka2 PE = 1 SV = 1 | | | | | | |
| Monocarboxylate transporter 4 | P57787 | Slc16a3 | 50 | kDa | 0 | 12 |
| OS = Mus musculus OX = 10090 | | | | | | |
| GN = Slc16a3 PE = 1 SV = 1 | | | | | | |
| Protein phosphatase 1 regulatory | Q99MR9 | Ppp1r3a | 121 | kDa | 0 | 12 |
| subunit 3A OS = Mus musculus | | | | | | |
| OX = 10090 GN = Ppp1r3a PE = 1 | | | | | | |
| SV = 2 | | | | | | |
| Dysferlin OS = Mus musculus | Q9ESD7 | Dysf | 238 | kDa | 0 | 12 |
| OX = 10090 GN = Dysf PE = 1 SV = 3 | | | | | | |
| Glypican-4 OS = Mus musculus | P51655 | Gpc4 | 63 | kDa | 0 | 11 |
| OX = 10090 GN = Gpc4 PE = 1 SV = 2 | | | | | | |
| Beta-sarcoglycan OS = Mus | P82349 | Sgcb | 35 | kDa | 0 | 9 |
| musculus OX = 10090 GN = Sgcb | | | | | | |
| PE = 1 SV = 1 | | | | | | |
| Leucine-rich PPR motif-containing | Q6PB66 | Lrpprc | 157 | kDa | 0 | 8 |
| protein, mitochondrial OS = Mus | | | | | | |
| musculus OX = 10090 GN = Lrpprc | | | | | | |
| PE = 1 SV = 2 | | | | | | |
| Collagen alpha-1(XV) chain | 035206 | Col15a1 | 140 | kDa | 0 | 7 |
| OS = Mus musculus OX = 10090 | | | | | | |
| GN = Col15a1 PE = 1 SV = 2 | | | | | | |
| Proliferation-associated protein | P50580 | Pa2g4 | 44 | kDa | 0 | 7 |
| 2G4 OS = Mus musculus | | | | | | |
| OX = 10090 GN = Pa2g4 PE = 1 | | | | | | |
| SV = 3 | | | | | | |
| Calcium/calmodulin-dependent | Q6PHZ2 | Camk2d | 56 | kDa | 0 | 7 |
| protein kinase type II subunit delta | | | | | | |
| OS = Mus musculus OX = 10090 | | | | | | |
| GN = Camk2d PE = 1 SV = 1 | | | | | | |
| Emerin OS = Mus musculus | O08579 | Emd | 29 | kDa | 0 | 6 |
| OX = 10090 GN = Emd PE = 1 SV = 1 | | | | | | |
| Glutamate dehydrogenase 1, | P26443 | Glud1 | 61 | kDa | 0 | 6 |
| mitochondrial OS = Mus musculus | | | | | | |
| OX = 10090 GN = Glud1 PE = 1 | | | | | | |
| SV = 1 | | | | | | |
| Transcriptional regulator ATRX | Q61687 | Atrx | 279 | kDa | 0 | 6 |
| OS = Mus musculus OX = 10090 | | | | | | |
| GN = Atrx PE = 1 SV = 3 | | | | | | |
| EH domain-containing protein 2 | Q8BH64 | Ehd2 | 61 | kDa | 0 | 6 |
| OS = Mus musculus OX = 10090 | | | | | | |
| GN = Ehd2 PE = 1 SV = 1 | | | | | | |
| BAG family molecular chaperone | Q91YN9 | Bag2 | 23 | kDa | 0 | 6 |
| regulator 2 OS = Mus musculus | | | | | | |
| OX = 10090 GN = Bag2 PE = 1 SV = 1 | | | | | | |
| C-type lectin domain family 3 | Q9EPW4 | Clec3a | 22 | kDa | 0 | 6 |
| member A OS = Mus musculus | | | | | | |
| OX = 10090 GN = Clec3a PE = 3 | | | | | | |
| SV = 1 | | | | | | |
| Delta-sarcoglycan OS = Mus | P82347 | Sgcd | 32 | kDa | 0 | 5 |
| musculus OX = 10090 GN = Sgcd | | | | | | |
| PE = 1 SV = 1 | | | | | | |
| Sorting and assembly machinery | Q8BGH2 | Samm50 | 52 | kDa | 0 | 5 |
| component 50 homolog OS = Mus | | | | | | |
| musculus OX = 10090 | | | | | | |
| GN = Samm50 PE = 1 SV = 1 | | | | | | |
| RUN and FYVE domain- | Q8BIJ7 | Rufy1 | 80 | kDa | 0 | 5 |
| containing protein 1 OS = Mus | | | | | | |
| musculus OX = 10090 GN = Rufy1 | | | | | | |
| PE = 1 SV = 1 | | | | | | |
| Acetyl-CoA acetyltransferase, | Q8QZT1 | Acat1 | 45 | kDa | 0 | 5 |
| mitochondrial OS = Mus musculus | | | | | | |
| OX = 10090 GN = Acat1 PE = 1 SV = 1 | | | | | | |
| Popeye domain-containing protein | Q9ES81 | Popdc3 | 34 | kDa | 0 | 5 |
| 3 OS = Mus musculus OX = 10090 | | | | | | |
| GN = Popdc3 PE = 1 SV = 1 | | | | | | |
| Gamma-sarcoglycan OS = Mus | P82348 | Sgcg | 32 | kDa | 0 | 5 |
| musculus OX = 10090 GN = Sgcg | | | | | | |
| PE = 1 SV = 2 | | | | | | |
| E3 ubiquitin-protein ligase UBR4 | A2AN08 | Ubr4 | 572 | kDa | 0 | 4 |
| OS = Mus musculus OX = 10090 | | | | | | |
| GN = Ubr4 PE = 1 SV = 1 | | | | | | |
| Band 3 anion transport protein | P04919 | Slc4a1 | 103 | kDa | 0 | 4 |
| OS = Mus musculus OX = 10090 | | | | | | |
| GN = Slc4a1 PE = 1 SV = 1 | | | | | | |
| Basigin OS = Mus musculus | P18572 | Bsg | 42 | kDa | 0 | 4 |
| OX = 10090 GN = Bsg PE = 1 SV = 2 | | | | | | |
| Long-chain specific acyl-CoA | P51174 | Acad1 | 48 | kDa | 0 | 4 |
| dehydrogenase, mitochondrial | | | | | | |
| OS = Mus musculus OX = 10090 | | | | | | |
| GN = Acad1 PE = 1 SV = 2 | | | | | | |
| Heterogeneous nuclear | P70333 | Hnrnph2 | 49 | kDa | 0 | 4 |
| ribonucleoprotein H2 OS = Mus | | | | | | |
| musculus OX = 10090 | | | | | | |
| GN = Hnrnph2 PE = 1 SV = 1 | | | | | | |
| DNA replication licensing factor | P97310 | Mcm2 | 102 | kDa | 0 | 4 |
| MCM2 OS = Mus musculus | | | | | | |
| OX = 10090 GN = Mcm2 PE = 1 | | | | | | |
| SV = 3 | | | | | | |
| Dystroglycan OS = Mus musculus | Q62165 | Dag1 | 97 | kDa | 0 | 4 |
| OX = 10090 GN = Dag1 PE = 1 SV = 4 | | | | | | |
| ATP-binding cassette sub-family F | Q6P542 | Abcf1 | 95 | kDa | 0 | 4 |
| member 1 OS = Mus musculus | | | | | | |
| OX = 10090 GN = Abcf1 PE = 1 | | | | | | |
| SV = 1 | | | | | | |
| Alanine--tRNA ligase, cytoplasmic | Q8BGQ7 | Aars1 | 107 | kDa | 0 | 4 |
| OS = Mus musculus OX = 10090 | | | | | | |
| GN = Aars1 PE = 1 SV = 1 | | | | | | |
| von Willebrand factor A domain- | Q8CC88 | Vwa8 | 213 | kDa | 0 | 4 |
| containing protein 8 OS = Mus | | | | | | |
| musculus OX = 10090 GN = Vwa8 | | | | | | |
| PE = 1 SV = 2 | | | | | | |
| 60S ribosomal protein L15 | Q9CZM2 | Rpl15 | 24 | kDa | 0 | 4 |
| OS = Mus musculus OX = 10090 | | | | | | |
| GN = Rpl15 PE = 1 SV = 4 | | | | | | |
| Dystrobrevin alpha OS = Mus | Q9D2N4 | Dtna | 84 | kDa | 0 | 4 |
| musculus OX = 10090 GN = Dtna | | | | | | |
| PE = 1 SV = 2 | | | | | | |
| NADH dehydrogenase | Q9DCT2 | Ndufs3 | 30 | kDa | 0 | 4 |
| [ubiquinone] iron-sulfur protein 3, | | | | | | |
| mitochondrial OS = Mus musculus | | | | | | |
| OX = 10090 GN = Ndufs3 PE = 1 | | | | | | |
| SV = 2 | | | | | | |
| Splicing factor 3A subunit 1 | Q8K4Z5 | Sf3a1 | 89 | kDa | 0 | 4 |
| OS = Mus musculus OX = 10090 | | | | | | |
| GN = Sf3a1 PE = 1 SV = 1 | | | | | | |
| Glutamine amidotransferase-like | Q9D172 | Gatd3a | 28 | kDa | 0 | 4 |
| class 1 domain-containing protein | | | | | | |
| 3A, mitochondrial OS = Mus | | | | | | |
| musculus OX = 10090 GN = Gatd3a | | | | | | |
| PE = 1 SV = 1 | | | | | | |
| Carboxypeptidase N catalytic chain | Q9JJN5 | Cpn1 | 52 | kDa | 0 | 4 |
| OS = Mus musculus OX = 10090 | | | | | | |
| GN = Cpn1 PE = 1 SV = 1 | | | | | | |
| ATP-sensitive inward rectifier | Q61743 | Kcnj11 | 44 | kDa | 0 | 4 |
| potassium channel 11 OS = Mus | | | | | | |
| musculus OX = 10090 GN = Kcnj11 | | | | | | |
| PE = 1 SV = 1 | | | | | | |
| Heparin cofactor 2 OS = Mus | P49182 | Serpind1 | 54 | kDa | 0 | 4 |
| musculus OX = 10090 | | | | | | |
| GN = Serpind1 PE = 1 SV = 1 | | | | | | |
| Talin-2 OS = Mus musculus | Q71LX4 | Tln2 | 254 | kDa | 0 | 3 |
| OX = 10090 GN = Tln2 PE = 1 SV = 3 | | | | | | |
| Sulfide:quinone oxidoreductase, | Q9R112 | Sqor | 50 | kDa | 0 | 3 |
| mitochondrial OS = Mus musculus | | | | | | |
| OX = 10090 GN = Sqor PE = 1 SV = 3 | | | | | | |
| Haloacid dehalogenase-like | Q3UGR5 | Hdhd2 | 29 | kDa | 0 | 3 |
| hydrolase domain-containing | | | | | | |
| protein 2 OS = Mus musculus | | | | | | |
| OX = 10090 GN = Hdhd2 PE = 1 | | | | | | |
| SV = 2 | | | | | | |
| NADH dehydrogenase | Q91YT0 | Ndufv1 | 51 | kDa | 0 | 3 |
| mitochondrial OS = Mus musculus | | | | | | |
| [ubiquinone] flavoprotein 1, | | | | | | |
| OX = 10090 GN = Ndufv1 PE = 1 | | | | | | |
| SV = 1 | | | | | | |
| Methylcrotonoyl-CoA carboxylase | Q99MR8 | Mccc1 | 79 | kDa | 0 | 3 |
| subunit alpha, mitochondrial | | | | | | |
| OS = Mus musculus OX = 10090 | | | | | | |
| GN = Mccc1 PE = 1 SV = 2 | | | | | | |
| Solute carrier family 12 member 2 | P55012 | Slc12a2 | 131 | kDa | 0 | 3 |
| OS = Mus musculus OX = 10090 | | | | | | |
| GN = Slc12a2 PE = 1 SV = 2 | | | | | | |
| Bifunctional purine biosynthesis | Q9CWJ9 | Atic | 64 | kDa | 0 | 3 |
| protein ATIC OS = Mus musculus | | | | | | |
| OX = 10090 GN = Atic PE = 1 SV = 2 | | | | | | |
| ATPase family AAA domain- | Q92511 | Atad3 | 67 | kDa | 0 | 3 |
| containing protein 3 OS = Mus | | | | | | |
| musculus OX = 10090 GN = Atad3 | | | | | | |
| PE = 1 SV = 1 | | | | | | |
| Cullin-2 OS = Mus musculus | Q9D4H8 | Cul2 | 87 | kDa | 0 | 3 |
| OX = 10090 GN = Cul2 PE = 1 SV = 2 | | | | | | |
| Dual specificity mitogen-activated | O09110 | Map2k3 | 39 | kDa | 0 | 3 |
| protein kinase kinase 3 OS = Mus | | | | | | |
| musculus OX = 10090 GN = Map2k3 | | | | | | |
| PE = 1 SV = 2 | | | | | | |
| Endoplasmic reticulum | Q9EPE9 | Atp13a1 | 132 | kDa | 0 | 3 |
| transmembrane helix translocase | | | | | | |
| OS = Mus musculus OX = 10090 | | | | | | |
| GN = Atp13a1 PE = 1 SV = 2 | | | | | | |
| Nucleolar and coiled-body | E9Q5C9 | Nolc1 | 74 | kDa | 0 | 3 |
| phosphoprotein 1 OS = Mus | | | | | | |
| musculus OX = 10090 GN = Nolc1 | | | | | | |
| PE = 1 SV = 1 | | | | | | |
| GTP-binding protein SAR1b | Q9CQC9 | Sar1b | 22 | kDa | 0 | 3 |
| OS = Mus musculus OX = 10090 | | | | | | |
| GN = Sar1b PE = 1 SV = 1 | | | | | | |
| Fibulin-1 OS = Mus musculus | Q08879 | Fbln1 | 78 | kDa | 0 | 3 |
| OX = 10090 GN = Fbln1 PE = 1 SV = 2 | | | | | | |
| Elongin-A OS = Mus musculus | Q8CB77 | Eloa | 87 | kDa | 0 | 3 |
| OX = 10090 GN = Eloa PE = 1 SV = 3 | | | | | | |
| Adenylate cyclase type 9 OS = Mus | | Adcy9 | 151 | kDa | 0 | 3 |
| musculus OX = 10090 GN = Adcy9 | P51830 | | | | | |
| PE = 1 SV = 1 | | | | | | |
| Barrier-to-autointegration factor | O54962 | Banf1 | 10 | kDa | 0 | 3 |
| OS = Mus musculus OX = 10090 | | | | | | |
| GN = Banf1 PE = 1 SV = 1 | | | | | | |
| Probable ubiquitin carboxyl- | P70398 | Usp9x | 291 | kDa | 0 | 3 |
| terminal hydrolase FAF-X | | | | | | |
| OS = Mus musculus OX = 10090 | | | | | | |
| GN = Usp9x PE = 1 SV = 2 | | | | | | |
| Sodium/calcium exchanger 3 | S4R2P9 | Slc8a3 | 103 | kDa | 0 | 3 |
| OS = Mus musculus OX = 10090 | | | | | | |
| GN = Slc8a3 PE = 1 SV = 1 | | | | | | |
| Eukaryotic initiation factor 4A-III | Q91VC3 | Eif4a3 | 47 | kDa | 0 | 3 |
| OS = Mus musculus OX = 10090 | | | | | | |
| GN = Eif4a3 PE = 1 SV = 3 | | | | | | |
| Fatty acid-binding protein, | P04117 | Fabp4 | 15 | kDa | 0 | 2 |
| adipocyte OS = Mus musculus | | | | | | |
| OX = 10090 GN = Fabp4 PE = 1 | | | | | | |
| SV = 3 | | | | | | |
| RNA methyltransferase 10 | Q3UFY8 | Trmt10c | 48 | kDa | 0 | 2 |
| homolog C OS = Mus musculus | | | | | | |
| OX = 10090 GN = Trmt10c PE = 1 | | | | | | |
| SV = 2 | | | | | | |
| Casein kinase II subunit alpha | Q60737 | Csnk2a1 | 45 | kDa | 0 | 2 |
| OS = Mus musculus OX = 10090 | | | | | | |
| GN = Csnk2a1 PE = 1 SV = 2 | | | | | | |
| C-1-tetrahydrofolate synthase, | Q922D8 | Mthfd1 | 101 | kDa | 0 | 2 |
| cytoplasmic OS = Mus musculus | | | | | | |
| OX = 10090 GN = Mthfd1 PE = 1 | | | | | | |
| SV = 4 | | | | | | |
| DnaJ homolog subfamily B | Q9QYI4 | Dnajb12 | 42 | kDa | 0 | 2 |
| member 12 OS = Mus musculus | | | | | | |
| OX = 10090 GN = Dnajb12 PE = 1 | | | | | | |
| SV = 2 | | | | | | |
| Serine-threonine kinase receptor- | Q9ZIZ2 | Strap | 38 | kDa | 0 | 2 |
| associated protein OS = Mus | | | | | | |
| musculus OX = 10090 GN = Strap | | | | | | |
| PE = 1 SV = 2 | | | | | | |
| Mannose-binding protein C | P41317 | Mb12 | 26 | kDa | 0 | 2 |
| OS = Mus musculus OX = 10090 | | | | | | |
| GN = Mb12 PE = 1 SV = 2 | | | | | | |
| Tetraspanin-8 OS = Mus musculus | Q8R3G9 | Tspan8 | 26 | kDa | 0 | 2 |
| OX = 10090 GN = Tspan8 PE = 1 | | | | | | |
| SV = 1 | | | | | | |
| ATP-binding cassette sub-family F | Q99LE6 | Abcf2 | 72 | kDa | 0 | 2 |
| member 2 OS = Mus musculus | | | | | | |
| OX = 10090 GN = Abcf2 PE = 1 | | | | | | |
| SV = 1 | | | | | | |
| Triggering receptor expressed on | O99NH8 | Trem2 | 25 | kDa | 0 | 2 |
| myeloid cells 2 OS = Mus musculus | | | | | | |
| OX = 10090 GN = Trem2 PE = 1 | | | | | | |
| SV = 1 | | | | | | |
| Charged multivesicular body | Q9D8B3 | Chmp4b | 25 | kDa | 0 | 2 |
| protein 4b OS = Mus musculus | | | | | | |
| OX = 10090 GN = Chmp4b PE = 1 | | | | | | |
| SV = 2 | | | | | | |
| Translocation protein SEC62 | Q8BU14 | Sec62 | 46 | kDa | 0 | 2 |
| OS = Mus musculus OX = 10090 | | | | | | |
| GN = Sec62 PE = 1 SV = 1 | | | | | | |
| Nuclear mitotic apparatus protein 1 | E9Q7G0 | Numa1 | 236 | kDa | 0 | 2 |
| OS = Mus musculus OX = 10090 | | | | | | |
| GN = Numa1 PE = 1 SV = 1 | | | | | | |
| Actin-binding LIM protein 2 | Q8BL65 | Ablim2 | 68 | kDa | 0 | 2 |
| OS = Mus musculus OX = 10090 | | | | | | |
| GN = Ablim2 PE = 1 SV = 1 | | | | | | |
| Nucleolar protein of 40 kDa | Q9ESX4 | Zcchc17 | 27 | kDa | 0 | 2 |
| OS = Mus musculus OX = 10090 | | | | | | |
| GN = Zcchc17 PE = 1 SV = 1 | | | | | | |
| Nuclear speckle splicing regulatory | Q5NCR9 | Nsrp1 | 64 | kDa | 0 | 2 |
| protein 1 OS = Mus musculus | | | | | | |
| OX = 10090 GN = Nsrp1 PE = 1 SV = 1 | | | | | | |
| DnaJ homolog subfamily B | Q9QYI5 | Dnajb2 | 36 | kDa | 0 | 2 |
| member 2 OS = Mus musculus | | | | | | |
| OX = 10090 GN = Dnajb2 PE = 1 | | | | | | |
| SV = 3 | | | | | | |
| Platelet glycoprotein 4 OS = Mus | Q08857 | Cd36 | 53 | kDa | 0 | 2 |
| musculus OX = 10090 GN = Cd36 | | | | | | |
| PE = 1 SV = 2 | | | | | | |
| Transmembrane protein 201 | A2A8U2 | Tmem201 | 73 | kDa | 0 | 2 |
| OS = Mus musculus OX = 10090 | | | | | | |
| GN = Tmem201 PE = 1 SV = 1 | | | | | | |
| Sarcospan OS = Mus musculus | Q62147 | Sspn | 24 | kDa | 0 | 2 |
| OX = 10090 GN = Sspn PE = 1 SV = 1 | | | | | | |
| F-box only protein 40 OS = Mus | P62932 | Fbxo40 | 80 | kDa | 0 | 2 |
| musculus OX = 10090 GN = Fbxo40 | | | | | | |
| PE = 1 SV = 1 | | | | | | |
| Multidrug resistance-associated | O35379 | Abcc1 | 171 | kDa | 0 | 2 |
| protein 1 OS = Mus musculus | | | | | | |
| OX = 10090 GN = Abcc1 PE = 1 | | | | | | |
| SV = 1 | | | | | | |
| Patatin-like phospholipase domain- | A2AJ88 | Pnpla7 | 150 | kDa | 0 | 2 |
| containing protein 7 OS = Mus | | | | | | |
| musculus OX = 10090 GN = Pnpla7 | | | | | | |
| PE = 1 SV = 1 | | | | | | |
| Myocilin OS = Mus musculus | O70624 | Myoc | 55 | kDa | 0 | 2 |
| OX = 10090 GN = Myoc PE = 1 SV = 1 | | | | | | |
| LEM domain-containing protein 2 | Q6DVA0 | Lemd2 | 58 | kDa | 0 | 2 |
| OS = Mus musculus OX = 10090 | | | | | | |
| GN = Lemd2 PE = 1 SV = 1 | | | | | | |
| Ran GTPase-activating protein 1 | P46061 | Rangap1 | 64 | kDa | 0 | 2 |
| OS = Mus musculus OX = 10090 | | | | | | |
| GN = Rangap1 PE = 1 SV = 2 | | | | | | |
| Cullin-1 OS = Mus musculus | Q9WTX6 | Cul1 | 90 | kDa | 0 | 2 |
| OX = 10090 GN = Cul1 PE = 1 SV = 1 | | | | | | |
| Coatomer subunit epsilon OS = Mus | O89079 | Cope | 35 | kDa | 0 | 2 |
| musculus OX = 10090 GN = Cope | | | | | | |
| PE = 1 SV = 3 | | | | | | |
| Microtubule-associated protein 4 | P27546 | Map4 | 117 | kDa | 0 | 2 |
| OS = Mus musculus OX = 10090 | | | | | | |
| GN = Map4 PE = 1 SV = 3 | | | | | | |
| mRNA turnover protein 4 homolog | Q9DO18 | Mrto4 | 28 | kDa | 0 | 2 |
| OS = Mus musculus OX = 10090 | | | | | | |
| GN = Mrto4 PE = 1 SV = 1 | | | | | | |
| NADH dehydrogenase | P52503 | Ndufs6 | 13 | kDa | 0 | 2 |
| [ubiquinone] iron-sulfur protein 6, | | | | | | |
| mitochondrial OS = Mus musculus | | | | | | |
| OX = 10090 GN = Ndufs6 PE = 1 | | | | | | |
| SV = 2 | | | | | | |
| 26S proteasome non-ATPase | P26516 | Psmd7 | 37 | kDa | 0 | 2 |
| regulatory subunit 7 OS = Mus | | | | | | |
| musculus OX = 10090 GN = Psmd7 | | | | | | |
| PE = 1 SV = 2 | | | | | | |
| CDP-diacylglycerol--inositol 3- | Q8VDP6 | Cdipt | 24 | kDa | 0 | 2 |
| phosphatidyltransferase OS = Mus | | | | | | |
| musculus OX = 10090 GN = Cdipt | | | | | | |
| PE = 1 SV = 1 | | | | | | |
| Coatomer subunit delta OS = Mus | Q5XJY5 | Arcn1 | 57 | kDa | 0 | 2 |
| musculus OX = 10090 GN = Arcn1 | | | | | | |
| PE = 1 SV = 2 | | | | | | |
| Transmembrane protein 43 | Q9DBS1 | Tmem43 | 45 | kDa | 0 | 2 |
| OS = Mus musculus OX = 10090 | | | | | | |
| GN = Tmem43 PE = 1 SV = 1 | | | | | | |
| Eukaryotic translation initiation | Q91WK2 | Eif3h | 40 | kDa | 0 | 2 |
| factor 3 subunit H OS = Mus | | | | | | |
| musculus OX = 10090 GN = Eif3h | | | | | | |
| PE = 1 SV = 1 | | | | | | |
| Medium-chain specific acyl-CoA | P45952 | Acadm | 46 | kDa | 0 | 2 |
| dehydrogenase, mitochondrial | | | | | | |
| OS = Mus musculus OX = 10090 | | | | | | |
| GN = Acadm PE = 1 SV = 1 | | | | | | |
| 40S ribosomal protein S27 | Q6ZWU9 | Rps27 | 9 | kDa | 0 | 2 |
| OS = Mus musculus OX = 10090 | | | | | | |
| GN = Rps27 PE = 1 SV = 3 | | | | | | |
| Heterogeneous nuclear | Q9DOE1 | Hnrnpm | 78 | kDa | 0 | 2 |
| ribonucleoprotein M OS = Mus | | | | | | |
| musculus OX = 10090 GN = Hnrnpm | | | | | | |
| PE = 1 SV = 3 | | | | | | |
| Vitamin K-dependent protein C | | Proc | 52 | kDa | 0 | 2 |
| OS = Mus musculus OX = 10090 | P33587 | | | | | |
| GN = Proc PE = 1 SV = 2 | | | | | | |
| Nesprin-2 OS = Mus musculus | Q6ZWQ0 | Syne2 | 783 | kDa | 0 | 2 |
| OX = 10090 GN = Syne2 PE = 1 | | | | | | |
| SV = 2 | | | | | | |
| Extended synaptotagmin-2 | Q3TZZ7 | Esyt2 | 94 | kDa | 0 | 2 |
| OS = Mus musculus OX = 10090 | | | | | | |
| GN = Esyt2 PE = 1 SV = 1 | | | | | | |
| Exosome complex exonuclease | Q9CSH3 | Dis3 | 109 | kDa | 0 | 2 |
| RRP44 OS = Mus musculus | | | | | | |
| OX = 10090 GN = Dis3 PE = 1 SV = 4 | | | | | | |
| Glutathione S-transferase | O89112 | Lancl1 | 45 | kDa | 0 | 2 |
| LANCL1 OS = Mus musculus | | | | | | |
| OX = 10090 GN = Lancl1 PE = 1 | | | | | | |
| SV = 1 | | | | | | |
| Lupus La protein homolog | P32067 | Ssb | 48 | kDa | 0 | 2 |
| OS = Mus musculus OX = 10090 | | | | | | |
| GN = Ssb PE = 1 SV = 1 | | | | | | |
| Phosphatidylinositol 5-phosphate | Q80XI4 | Pip4k2b | 47 | kDa | 0 | 2 |
| 4-kinase type-2 beta OS = Mus | (+1) | | | | | |
| musculus OX = 10090 GN = Pip4k2b | | | | | | |
| PE = 1 SV = 1 | | | | | | |
| Flavin reductase (NADPH) | | Blvrb | 22 | kDa | 0 | 2 |
| OS = Mus musculus OX = 10090 | Q923D2 | | | | | |
| GN = Blvrb PE = 1 SV = 3 | | | | | | |
| Heterogeneous nuclear | Q8R081 | Hnrnpl | 64 | kDa | 0 | 2 |
| ribonucleoprotein L OS = Mus | | | | | | |
| musculus OX = 10090 GN = Hnrnpl | | | | | | |
| PE = 1 SV = 2 | | | | | | |
| Cell cycle progression protein 1 | Q640L3 | Ccpg1 | 86 | kDa | 0 | 2 |
| OS = Mus musculus OX = 10090 | | | | | | |
| GN = Ccpg1 PE = 1 SV = 2 | | | | | | |
| U2 small nuclear ribonucleoprotein | Q9CQI7 | Snrpb2 | 25 | kDa | 0 | 2 |
| B″ OS = Mus musculus OX = 10090 | | | | | | |
| GN = Snrpb2 PE = 1 SV = 1 | | | | | | |
| Phosphofurin acidic cluster sorting | Q8K212 | Pacs1 | 105 | kDa | 0 | 2 |
| protein 1 OS = Mus musculus | | | | | | |
| OX = 10090 GN = Pacs1 PE = 1 SV = 2 | | | | | | |
| Anoctamin-5 OS = Mus musculus | Q75UR0 | Ano5 | 106 | kDa | 0 | 2 |
| OX = 10090 GN = Ano5 PE = 2 SV = 1 | | | | | | |
| sp\|Q6P5D8\|SMHD1_MOUSE- | sp\|Q6P5D8\| | | ? | | 0 | 2 |
| DECOY | SMHD1_MOUSE- | | | | | |
| | DECOY | | | | | |
| N-alpha-acetyltransferase 50 | Q6PGB6 | Naa50 | 19 | kDa | 0 | 2 |
| OS = Mus musculus OX = 10090 | | | | | | |
| GN = Naa50 PE = 1 SV = 1 | | | | | | |
| Sodium/calcium exchanger 1 | P70414 | Slc8a1 | 108 | kDa | 0 | 2 |
| OS = Mus musculus OX = 10090 | | | | | | |
| GN = Slc8a1 PE = 1 SV = 1 | | | | | | |
| U1 small nuclear ribonucleoprotein | Q62376 | Snrnp70 | 52 | kDa | 0 | 2 |
| 70 kDa OS = Mus musculus | | | | | | |
| OX = 10090 GN = Snrnp70 PE = 1 | | | | | | |
| SV = 2 | | | | | | |
| Indolethylamine N- | P40936 | Inmt | 29 | kDa | 0 | 2 |
| methyltransferase OS = Mus | | | | | | |
| musculus OX = 10090 GN = Inmt | | | | | | |
| PE = 1 SV = 1 | | | | | | |
| Solute carrier family 12 member 1 | P55014 | Slc12a1 | 120 | kDa | 0 | 2 |
| OS = Mus musculus OX = 10090 | | | | | | |
| GN = Slc12a1 PE = 1 SV = 2 | | | | | | |
| MAP7 domain-containing protein | A2AJI0 | Map7d1 | 93 | kDa | 0 | 2 |
| 1 OS = Mus musculus OX = 10090 | | | | | | |
| GN = Map7d1 PE = 1 SV = 1 | | | | | | |
| Obg-like ATPase 1 OS = Mus | Q9CZ30 | Ola1 | 45 | kDa | 0 | 2 |
| musculus OX = 10090 GN = Ola1 | | | | | | |
| PE = 1 SV = 1 | | | | | | |
| NEDD8-conjugating enzyme | P61082 | Ube2m | 21 | kDa | 0 | 2 |
| Ubc12 OS = Mus musculus | | | | | | |
| OX = 10090 GN = Ube2m PE = 1 | | | | | | |
| SV = 1 | | | | | | |
| Nucleolar protein 12 OS = Mus | Q8BG17 | Nol12 | 25 | kDa | 0 | 2 |
| musculus OX = 10090 GN = Nol12 | | | | | | |
| PE = 1 SV = 1 | | | | | | |
| sp\|Q60952\|CP250_MOUSE- | sp\|Q60952\| | | ? | | 0 | 2 |
| DECOY | CP250_MOUSE- | | | | | |
| | DECOY | | | | | |
| Chromatin-remodeling ATPase | Q6ZPV2 | Ino80 | 177 | kDa | 0 | 2 |
| INO80 OS = Mus musculus | | | | | | |
| OX = 10090 GN = Ino80 PE = 1 SV = 2 | | | | | | |
| sp\|Q8CJ40\|CROCC_MOUSE- | sp\|Q8CJ40\| | | ? | | 0 | 2 |
| DECOY | CROCC_MOUSE- | | | | | |
| | DECOY | | | | | |
| Ferrochelatase, mitochondrial | P22315 | Fech | 47 | kDa | 0 | 2 |
| OS = Mus musculus OX = 10090 | | | | | | |
| GN = Fech PE = 1 SV = 2 | | | | | | |
| Ribosomal protein S6 kinase alpha- | P18654 | Rps6ka3 | 84 | kDa | 1 | 20 |
| 3 OS = Mus musculus OX = 10090 | | | | | | |
| GN = Rps6ka3 PE = 1 SV = 2 | | | | | | |
| Cullin-associated NEDD8- | Q6ZQ73 | Cand2 | 136 | kDa | 1 | 16 |
| dissociated protein 2 OS = Mus | | | | | | |
| musculus OX = 10090 GN = Cand2 | | | | | | |
| PE = 1 SV = 2 | | | | | | |
| Caveolae-associated protein 2 | Q63918 | Cavin2 | 47 | kDa | 1 | 12 |
| OS = Mus musculus OX = 10090 | | | | | | |
| GN = Cavin2 PE = 1 SV = 3 | | | | | | |
| Glypican-1 OS = Mus musculus | Q9QZF2 | Gpc1 | 61 | kDa | 1 | 11 |
| OX = 10090 GN = Gpc1 PE = 1 SV = 1 | | | | | | |
| Alpha-1-syntrophin OS = Mus | Q61234 | Snta1 | 54 | kDa | 1 | 11 |
| musculus OX = 10090 GN = Snta1 | | | | | | |
| PE = 1 SV = 1 | | | | | | |
| Caveolae-associated protein 3 | Q91VJ2 | Cavin3 | 28 | kDa | 1 | 11 |
| OS = Mus musculus OX = 10090 | | | | | | |
| GN = Cavin3 PE = 1 SV = 1 | | | | | | |
| Caveolin-1 OS = Mus musculus | P49817 | Cav1 | 21 | kDa | 1 | 8 |
| OX = 10090 GN = Cav1 PE = 1 SV = 1 | | | | | | |
| Unconventional myosin-Ic | Q9WTI7 | Myo1c | 122 | kDa | 1 | 7 |
| OS = Mus musculus OX = 10090 | | | | | | |
| GN = Myo1c PE = 1 SV = 2 | | | | | | |
| Collagen alpha-2(IV) chain | P08122 | Col4a2 | 167 | kDa | 1 | 5 |
| OS = Mus musculus OX = 10090 | | | | | | |
| GN = Col4a2 PE = 1 SV = 4 | | | | | | |
| Protein-cysteine N- | Q9D1G3 | Hhatl | 56 | kDa | 1 | 5 |
| palmitoyltransferase HHAT-like | | | | | | |
| protein OS = Mus musculus | | | | | | |
| OX = 10090 GN = Hhatl PE = 1 SV = 2 | | | | | | |
| Alpha-sarcoglycan OS = Mus | P82350 | Sgca | 43 | kDa | 1 | 5 |
| musculus OX = 10090 GN = Sgca | | | | | | |
| PE = 1 SV = 1 | | | | | | |
| Vinculin OS = Mus musculus | Q64727 | Vcl | 117 | kDa | 1 | 5 |
| OX = 10090 GN = Vcl PE = 1 SV = 4 | | | | | | |
| Spectrin beta chain, erythrocytic | P15508 | Sptb | 245 | kDa | 2 | 41 |
| OS = Mus musculus OX = 10090 | | | | | | |
| GN = Sptb PE = 1 SV = 4 | | | | | | |
| Caveolae-associated protein 1 | O54724 | Cavin1 | 44 | kDa | 2 | 19 |
| OS = Mus musculus OX = 10090 | | | | | | |
| GN = Cavin1 PE = 1 SV = 1 | | | | | | |
| Calcium/calmodulin-dependent | P11798 | Camk2a | 54 | kDa | 2 | 11 |
| protein kinase type II subunit alpha | | | | | | |
| OS = Mus musculus OX = 10090 | | | | | | |
| GN = Camk2a PE = 1 SV = 2 | | | | | | |
| Serine/arginine repetitive matrix | Q8BTI8 | Srrm2 | 295 | kDa | 2 | 11 |
| protein 2 OS = Mus musculus | | | | | | |
| OX = 10090 GN = Srrm2 PE = 1 | | | | | | |
| SV = 3 | | | | | | |
| Very-long-chain enoyl-CoA | Q9CY27 | Tecr | 36 | kDa | 2 | 9 |
| reductase OS = Mus musculus | | | | | | |
| OX = 10090 GN = Tecr PE = 1 SV = 1 | | | | | | |
| Succinate--CoA ligase [ADP- | Q9Z219 | Sucla2 | 50 | kDa | 2 | 8 |
| forming] subunit beta, | | | | | | |
| mitochondrial OS = Mus musculus | | | | | | |
| OX = 10090 GN = Sucla2 PE = 1 | | | | | | |
| SV = 2 | | | | | | |
| PDZ and LIM domain protein 5 | Q8CI51 | Pdlim5 | 63 | kDa | 2 | 7 |
| OS = Mus musculus OX = 10090 | | | | | | |
| GN = Pdlim5 PE = 1 SV = 4 | | | | | | |
| Trimeric intracellular cation | Q3TMP8 | Tmem38a | 33 | kDa | 2 | 7 |
| channel type A OS = Mus musculus | | | | | | |
| OX = 10090 GN = Tmem38a PE = 1 | | | | | | |
| SV = 2 | | | | | | |
| Poly(rC)-binding protein 2 | Q61990 | Pcbp2 | 38 | kDa | 2 | 6 |
| OS = Mus musculus OX = 10090 | | | | | | |
| GN = Pcbp2 PE = 1 SV = 1 | | | | | | |
| T-complex protein 1 subunit beta | P80314 | Cct2 | 57 | kDa | 2 | 6 |
| OS = Mus musculus OX = 10090 | | | | | | |
| GN = Cct2 PE = 1 SV = 4 | | | | | | |
| Phosphorylase b kinase gamma | P07934 | Phkg1 | 45 | kDa | 2 | 6 |
| catalytic chain, skeletal | | | | | | |
| muscle/heart isoform OS = Mus | | | | | | |
| musculus OX = 10090 GN = Phkg1 | | | | | | |
| PE = 1 SV = 3 | | | | | | |
| Voltage-dependent L-type calcium | Q8R3Z5 | Cacnb1 | 65 | kDa | 2 | 6 |
| channel subunit beta-1 OS = Mus | | | | | | |
| musculus OX = 10090 GN = Cacnb1 | | | | | | |
| PE = 1 SV = 1 | | | | | | |
| Tripartite motif-containing protein | Q1XH17 | Trim72 | 53 | kDa | 2 | 6 |
| 72 OS = Mus musculus OX = 10090 | | | | | | |
| GN = Trim72 PE = 1 SV = 1 | | | | | | |
| Transcription elongation factor A | P23881 | Tcea3 | 39 | kDa | 3 | 12 |
| protein 3 OS = Mus musculus | | | | | | |
| OX = 10090 GN = Tcea3 PE = 1 SV = 3 | | | | | | |
| Cytoplasmic dynein 1 heavy chain | Q9JHU4 | Dync1h1 | 532 | kDa | 3 | 12 |
| 1 OS = Mus musculus OX = 10090 | | | | | | |
| GN = Dync1h1 PE = 1 SV = 2 | | | | | | |
| Nidogen-2 OS = Mus musculus | O88322 | Nid2 | 154 | kDa | 3 | 12 |
| OX = 10090 GN = Nid2 PE = 1 SV = 2 | | | | | | |
| Perilipin-4 OS = Mus musculus | O88492 | Plin4 | 139 | kDa | 3 | 11 |
| OX = 10090 GN = Plin4 PE = 1 SV = 2 | | | | | | |
| 3-ketoacyl-CoA thiolase, | Q8BWTI | Acaa2 | 42 | kDa | 3 | 9 |
| mitochondrial OS = Mus musculus | | | | | | |
| OX = 10090 GN = Acaa2 PE = 1 | | | | | | |
| SV = 3 | | | | | | |
| Myosin light chain kinase 2, | O8VCR8 | Mylk2 | 66 | kDa | 3 | 9 |
| skeletal/cardiac muscle OS = Mus | | | | | | |
| musculus OX = 10090 GN = Mylk2 | | | | | | |
| PE = 1 SV = 2 | | | | | | |
| Carnitine O-palmitoyltransferase 1, | Q924X2 | Cpt1b | 88 | kDa | 4 | 24 |
| muscle isoform OS = Mus musculus | | | | | | |
| OX = 10090 GN = Cpt1b PE = 1 SV = 1 | | | | | | |
| Phosphorylase b kinase regulatory | Q7TSH2 | Phkb | 124 | kDa | 4 | 18 |
| subunit beta OS = Mus musculus | | | | | | |
| OX = 10090 GN = Phkb PE = 1 SV = 1 | | | | | | |
| MICOS complex subunit Mic60 | Q8CAQ8 | Immt | 84 | kDa | 4 | 15 |
| OS = Mus musculus OX = 10090 | | | | | | |
| GN = Immt PE = 1 SV = 1 | | | | | | |
| Trifunctional enzyme subunit beta, | Q99JY0 | Hadhb | 51 | kDa | 4 | 13 |
| mitochondrial OS = Mus musculus | | | | | | |
| OX = 10090 GN = Hadhb PE = 1 | | | | | | |
| SV = 1 | | | | | | |
| Spectrin alpha chain, non- | P16546 | Sptan1 | 285 | kDa | 5 | 24 |
| erythrocytic 1 OS = Mus musculus | | | | | | |
| OX = 10090 GN = Sptan1 PE = 1 | | | | | | |
| SV = 4 | | | | | | |
| Phosphorylase b kinase regulatory | P18826 | Phka1 | 139 | kDa | 5 | 23 |
| subunit alpha, skeletal muscle | | | | | | |
| isoform OS = Mus musculus | | | | | | |
| OX = 10090 GN = Phka1 PE = 1 | | | | | | |
| SV = 3 | | | | | | |
| Spectrin beta chain, non- | Q62261 | Sptbn1 | 274 | kDa | 5 | 19 |
| erythrocytic 1 OS = Mus musculus | | | | | | |
| OX = 10090 GN = Sptbn1 PE = 1 | | | | | | |
| SV = 2 | | | | | | |
| Annexin A6 OS = Mus musculus | P14824 | Anxa6 | 76 | kDa | 5 | 17 |
| OX = 10090 GN = Anxa6 PE-1 | | | | | | |
| SV = 3 | | | | | | |
| Biglycan OS = Mus musculus | P28653 | Bgn | 42 | kDa | 5 | 16 |
| OX = 10090 GN = Bgn PE = 1 SV = 1 | | | | | | |
| Striated muscle-specific | Q62407 | Speg | 354 | kDa | 5 | 16 |
| serine/threonine-protein kinase | | | | | | |
| OS = Mus musculus OX = 10090 | | | | | | |
| GN = Speg PE = 1 SV = 2 | | | | | | |
| Eukaryotic translation initiation | Q05D44 | Eif5b | 138 | kDa | 5 | 14 |
| factor 5B OS = Mus musculus | | | | | | |
| OX = 10090 GN = Eif5b PE = 1 SV = 2 | | | | | | |
| Dystrophin OS = Mus musculus | P11531 | Dmd | 426 | kDa | 6 | 77 |
| OX = 10090 GN = Dmd PE = 1 SV = 3 | | | | | | |
| Sodium/potassium-transporting | Q6PIE5 | Atpla2 | 112 | kDa | 6 | 41 |
| ATPase subunit alpha-2 OS = Mus | | | | | | |
| musculus OX = 10090 GN = Atpla2 | | | | | | |
| PE = 1 SV = 1 | | | | | | |

TABLE 2

| Gene/ | Forward primer | Reverse primer |
|---|---|---|
| Name | (5′->3′) | (5′->3′) |
| Atrogin | TAGTAAGGCTGTTGG | CTGCACCAGTGTGCA |
| 1 | AGCTGATAG | TAAGG |
| | (SEQ ID NO: 13) | (SEQ ID NO: 14) |
| MuRF1 | CATCTTCCAGGCTGC | ACTGGAGCACTCCTG |
| | GAATC | CTTGT |
| | (SEQ ID NO: 15) | (SEQ ID NO: 16) |
| MUSA1 | CTTCAGTCTCGTGGA | TGCAGTACTGAATCG |
| | ATGGTAATCTT | CCATAC |
| | (SEQ ID NO: 17) | (SEQ ID NO: 18) |
| Bnip3 | TCCTGGGTAGAACTG | GCTGGGCATCCAACA |
| | CACTTC | GTATTT |
| | (SEQ ID NO: 19) | (SEQ ID NO: 20) |
| p62 | AGGATGGGGACTTGG | TCACAGATCACATTG |
| | TTGC | GGGTGC |
| | (SEQ ID NO: 21) | (SEQ ID NO: 22) |
| Beclin1 | TGGAAGGGTCTAAGA | GGCTGTGGTAAGTAA |
| | CGT | TGGA |
| | (SEQ ID NO: 23) | (SEQ ID NO: 24) |
| Cts1 | GTGGACTGTTCTCAC | TCCGTCCTTCGCTTC |
| | GCTCAAG | ATAGG |
| | (SEQ ID NO: 25) | (SEQ ID NO: 26) |
| GAPDH | AGGTCGGTGTGAACG | TGTAGACCATGTAGT |
| | GATTTG | TGAGGTCA |
| | (SEQ ID NO: 27) | (SEQ ID NO: 28) |
| BVES | CCGTGCCTTCTAATG | AGACCACATAAAGGG |
| | AGACCA | TACATCCT |
| | (SEQ ID NO: 29) | (SEQ ID NO: 30) |
| POPDC2 | AGGAGCTGGAAACCG | GACGATGTCTAGTCC |
| | GATGTA | ACAAGC |
| | (SEQ ID NO: 31) | (SEQ ID NO: 32) |
| POPDC3 | TGACTGAACACCCAC | ACTGCCACCCATAAA |
| | TCTGC | ACCTACT |
| | (SEQ ID NO: 33) | (SEQ ID NO: 34) |
| ATG7 | TCTGGGAAGCCATAA | GCGAAGGTCAGGAGC |
| | AGTCAGG | AGAA |
| | (SEQ ID NO: 35) | (SEQ ID NO: 36) |
| PARK2 | TCTTCCAGTGTAACC | GGCAGGGAGTAGCCA |
| | ACCGTC | AGTT |
| | (SEQ ID NO: 37) | (SEQ ID NO: 38) |
| hADCY3- | ACCGCGTACCTGACG | AAACTCACGAGAACG |
| E4gRNA | TTCTCGTGA | TCAGGTACG |
| | (SEQ ID NO: 39) | (SEQ ID NO: 40) |
| hADCY3 | ACCTCATGCTTTCCA | CCGCTCCAGGTGATA |
| | TCCTG | TCTGT |
| | (SEQ ID NO: 41) | (SEQ ID NO: 42) |
| hADCY6- | ACCGCCGCCAGGACT | AAACCCCACATGGAG |
| Q205gRNA | CCATGTGGG | TCCTGGCGG |
| | (SEQ ID NO: 43) | (SEQ ID NO: 44) |
| hADCY6 | AGGATACCGAGGTGA | GCCAAGATCAAATGC |
| | CAACG | AAGGT |
| | (SEQ ID NO: 45) | (SEQ ID NO: 46) |
| Bves-KO | ACTTGCTTTAAAAAA | AGTCACTAGCAAGAG |
| | CCTCCCACA | ATCTGCACCC |
| | (SEQ ID NO: 9) | (SEQ ID NO: 10) |
| Bves-WT | AAGTGCTGGGATTA | AAGGACACATCACAG |
| | AAGGTGTGTGC | CTTCAGG |
| | (SEQ ID NO: 11) | (SEQ ID NO: 12) |

TABLE 3

| | | |
|---|---|---|
| Rabbit anti-BVES | Sigma-Aldrich #HPA014788 | WB(1:500), |
| | | IF(1:50) |
| Rabbit anti-ADCY9 | Abcam #ab191423 | WB(1:1000) |
| Rabbit anti-HA | Cell Signaling Technology #3724 | WB(1:1000), |
| | | IF(1:200) |
| Rabbit anti-Phospho-PKA | Cell Signaling Technology #9624 | WB(1:1000) |
| Substrate (RRXS*/T*) | | |
| Rabbit anti-LC3A/B | Cell Signaling Technology #4108 | WB(1:1000) |
| Rabbit anti-LC3B | Proteintech #18725-1-AP | IF(1:50) |
| Rabbit anti-FIP200 | Cell Signaling Technology #12436 | WB(1:1000) |
| Rabbit anti-VAMP7 | Proteintech #22268-1-AP | WB(1:500) |
| Mouse anti-Ubiquitin | Santa Cruz #sc-8017 | WB(1:500) |
| Rabbit anti-phospho-4E- | Cell Signaling Technology #2855 | WB(1:1000) |
| BP1 (Thr37/46) | | |
| Rabbit anti-4E-BP1 | Cell Signaling Technology #9644 | WB(1:1000) |
| Rabbit anti-AKT | Cell Signaling Technology #9272 | WB(1:1000) |
| Rabbit anti-phospho-Akt | Cell Signaling Technology #4060 | WB(1:1000) |
| (Ser473) | | |
| Rabbit anti-phospho-Akt | Cell Signaling Technology #9275 | WB(1:500) |
| (Thr308) | | |
| Rabbit anti-phospho-p70 | Cell Signaling Technology #9205 | WB(1:500) |
| S6 Kinase (Thr389) | | |
| Rabbit anti-p70 S6 Kinase | Cell Signaling Technology #9202 | WB(1:1000) |

anti-phospho-

| | | |
|---|---|---|
| Rabbit anti-phospho- | Cell Signaling Technology #9721 | WB(1:1000) |
| eIF2a (Ser51) | | |
| Rabbit anti-eIF2a | Cell Signaling Technology #9722 | WB(1:1000) |
| Rabbit anti-15- | Novus Biologicals #NB200-179 | WB(1:1000) |
| PGDH/HPGD | | |
| Mouse anti-Cox-2 | Santa Cruz #sc-376861 | WB(1:1000) |
| Rabbit anti-Na, K-ATPase | Cell Signaling Technology #3010 | WB(1:1000) |
| Rabbit anti-dystrophin | Spring Bioscience #E2660 | IF(1:200) |
| Mouse anti-MHC2a | Developmental Studies Hybridoma Bank | |
| (A4.74) | #AB 528383 | IF(1:50) |
| Mouse anti-MHC2b | Developmental Studies Hybridoma Bank | IF(1:50) |
| (10F5) | #AB 1157896 | |
| Mouse Anti-Caveolin 3 | BD #610420 | IF(1:400) |
| mouse anti-puromycin | Sigma-Aldrich #MABE343 | WB(1:1000) |
| clone 12D10 | | |
| Rabbit anti-ATG16L | Cell Signaling Technology #8089 | WB(1:1000) |
| Rabbit anti-ATG5 | Cell Signaling Technology #12994 | WB(1:1000) |
| Rabbit anti-ATG9a | Cell Signaling Technology #13509 | WB(1:1000) |
| Rabbit anti-VPS34 | Cell Signaling Technology #4263 | WB(1:1000) |
| Rabbit anti-VPS15 | Cell Signaling Technology #14580 | WB(1:1000) |
| Rabbit anti-Rab7 | Cell Signaling Technology #9367 | WB(1:1000) |
| Rabbit anti-LAMP1 | Cell Signaling Technology #9091 | WB(1:1000) |
| Rabbit anti-STX17 | Proteintech #17815-1-AP | WB(1:500) |

```
SEQUENCES
  1. SEQ ID NO: 1 - BVES
ATGAATTATACAGAGTCCAGCCCATTGAGAGAATCAACTGCCATAGGTTTTACACCTG
AGTTAGAAAGTATCATACCTGTGCCTTCCAATAAGACCACTTGTGAAAACTGGAGAGA
GATACATCATCTGGTTTTTCATGTAGCAAATATTTGTTTTGCAGTTGGGTTGGTTATTCC
AACTACTCTTCACCTTCATATGATATTTCTTAGGGGAATGTTAACTCTAGGATGTACCCT
TTATATCGTCTGGGCCACTCTCTACCGATGTGCCTTGGATATAATGATCTGGAACTCTG
TGTTCTTGGGTGTCAACATTTTGCATCTGTCGTATCTTTTATACAAGAAGAGACCGGTA
AAGATTGAAAAGGAACTCAGTGGCATGTACCGGCGATTGTTTGAACCACTCCGTGTGC
CTCCAGATTTGTTCAGAAGACTAACTGGACAGTTTTGCATGATCCAAACCTTGAAAAAG
GGCCAAACTTATGCTGCAGAGGATAAAACCTCAGTTGATGACCGTCTGAGTATTCTCTT
GAAGGGAAAAATGAAGGTCTCCTATCGAGGACATTTTCTGCATAACATTTACCCCTGTG
CCTTTATAGATTCTCCTGAATTTAGATCAACTCAGATGCACAAAGGTGAAAAATTCCAG
GTCACCATTATTGCAGATGATAACTGCAGATTTTTATGCTGGTCAAGAGAAAGATTAAC
ATACTTTCTGGAATCAGAACCTTTCTTGTATGAAATCTTTAGGTATCTTATTGGAAAAG
ACATCACAAATAAGCTCTACTCATTGAATGATCCCACCTTAAATGATAAAAAAGCCAA
AAAGCTGGAACATCAGCTCAGCCTCTGCACACAGATCTCCATGTTGGAAATGAGGAAC
AGTATAGCCAGCTCCAGTGACAGTGACGACGGCTTGCACCAGTTTCTTCGGGGTACCTC
CAGCATGTCCTCTCTTCATGTGTCATCCCCACACCAGCGAGCCTCTGCCAAGATGAAAC
CGATAGAAGAAGGAGCAGAAGATGATGATGACGTTTTTGAACCGGCATCTCCAAATAC
ATTGAAAGTCCATCAGCTGCCT
  2. SEQ ID NO: 2 - BVES Amino acids
MNYTESSPLRESTAIGFTPELESIIPVPSNKTTCENWREIHHLVFHVANICFAVGLVIPTTLHL
HMIFLRGMLTLGCTLYIVWATLYRCALDIMIWNSVFLGVNILHLSYLLYKKRPVKIEKELS
GMYRRLFEPLRVPPDLFRRLTGQFCMIQTLKKGQTYAAEDKTSVDDRLSILLKGKMKVSY
RGHFLHNIYPCAFIDSPEFRSTQMHKGEKFQVTIIADDNCRFLCWSRERLTYFLESEPFLYEIF
RYLIGKDITNKLYSLNDPTLNDKKAKKLEHQLSLCTQISMLEMRNSIASSSDSDDGLHQFLR
GTSSMSSLHVSSPHQRASAKMKPIEEGAEDDDDVFEPASPNTLKVHQLP
  3. SEQ ID NO: 3 - MHCK7
CCCTTCAGATTAAAAATAACTGAGGTAAGGGCCTGGGTAGGGGAGGTGGTGTGAGACG
CTCCTGTCTCTCCTCTATCTGCCCATCGGCCCTTTGGGGAGGAGGAATGTGCCCAAGGA
CTAAAAAAAGGCCATGGAGCCAGAGGGGCGAGGGCAACAGACCTTTCATGGGCAAAC
CTTGGGGCCCTGCTGactgtaGATGAGAGCAGCCACTACGGGTCTAGGCTGCCCATGTAAG
GAGGCAAGGCCTGGGGACACCCGAGATGCCTGGTTATAATTAACCCAGACATGTGGCT
GCCCCCCCCCCCCCAACACCTGCTGCCTGCTAAAAATAACCCTGTCCCTGGTGGccctgcat
gcccTTCGAACAAGGCTGTGGGGGACTGAGGGCAGGCTGTAACAGGCTTGGGGGCCAGG
GCTTATACGTGCCTGGGACTCCCAAAGTATTACTGTTCCATGTTCCCGGCGAAGGGCCA
GCTGTCCCCCGCCAGCTAGACTCAGCACTTAGTTTAGGAACCAGTGAGCAAGTCAGCC
CTTGGGGCAGCCCATACAAGGCCATGGGGCTGGGCAAGCTGCACGCCTGGGTCCGGGG
TGGGCACGGTGCCCGGGCAACGAGCTGAAAGCTCATCTACTCTCAGGGGCCCCTCCCT
GGGGACAGCCCCTCCTGGCTAGTCACACCCTGTAGGCTCCTCTATATAACCCAGGGGC
ACAGGGGCTGCCCtcattctACCACCACCTCCACAGCACAGACAGACACTCAGGAGCCAGC
  4. SEQ ID NO: 4 - ITR
aggaacccctagtgatggagttggccactccctctctgcgcgctcgctcgctcactgaggccgg
gcgaccaaaggtcgcccgacgcccgggctttgcccgggcggcctcagtgagcgagcgagcgcgc
agctgcctgcagg
  5. SEQ ID NO: 5 - 3xHA
tacccatacgatgttccagattacgcttacccatacgatgttccagattacgcttacccatacg
atgttccagattacgct
  6. SEQ ID NO: 6 - 3xHA Amino acids
YPYDVPDYAYPYDVPDYAYPYDVPDYA
  7. SEQ ID NO: 7 - bGH_poly A
cgactgtgccttctagttgccagccatctgttgtttgcccctcccccgtgccttccttgaccct
ggaaggtgccactcccactgtcctttcctaataaaatgaggaaattgcatcgcattgtctgagt
aggtgtcattctattctggggggtggggggggcaggacagcaagggggaggattgggaagagaa
tagcaggcatgctgggga
  8. SEQ ID NO: 8 - pX601-stuffer-MHCK7-hBVES-3xHA
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggcctctagaatttccttcaaaattgtttttaaaggcctatttaatatcttaccctatct
attccagcctgggcaacatagcaagatcctgtctctacaaaaaaaaaaaataaaaaattagttg
ggcatggtgcctgtagtccagctacctgagaggccaaagcaggaggattgctggagcccaggag
tttcatggttgtgctgctgcactccagccttgccaacagagtgacgctctgtcttaaagaaaaa
gaaaagaaaaacagaagcattactctctagttctcccattttcagtgaactaataaattattat
accacacaatccataatttctcctatgccatgaacctttgttacctaaagttcttcctcattgt
gtaggctaaacaaagaagctggGTaagaagaGTgagtacgttttacagtttatatgtatcactt
atataacGTaagtaaaattttgagcttatagctagttactaaaaacttaagaaattgcccagaa
ccgcataggttattgtggatttagagacgagtatacattgaccatgaaatatatgtttaaaaat
agtctttctctgcatgtgtctttgtatgtctctctttctgtctttcacatatatacacacatta
tatgtatacatatatacatgtttttgtaactatatgttttgtgaattttgttttctcaaagttt
tgcctgaaaatttcaaaatattgaaaatgctttgatgtgtttgagattttataaaatattatcc
atcttatttaatcagctcaataacaaactgatcagtaagctttctgctgttttgcctttttttt
aatgcagGACTCGGAAGATGGAAGAACTTATTTTGTCAAGATCCATGCCCCTTGGGAGGTATTA
GTTACCTATGCTGAAGTCTTGGGAATCAAAATGCCTATTAAGGAGAGTGATATTCCCCGCCCTA
AGCACACTCCTATAAGCTATGTGCTTGGACCTGTAAGACTCCCACTGAGTGTGAAGTATCCCCA
TCCTGAATATTTTACTGCACAATTCAGCAGACATCGGCAGGAGCTCTTCCTCATCGAAGATCAG
GCAACCTTCTTTCCATCCTCATCAAGAAACAGAATTGTGTACTATATTCTCTCAAGATGT
CCTTTTGGCATAGAAGATGGGAAGAAAAGGTTTGGGATTGAAAGACTGCTAAACTCTA
ACACTTACTCATCTGCCTATCCACTCCATGATGGCCAATATTGGAAGCCATCAGAACCT
CCCAATCCTACCAATGAAAGATACACACTTCACCAGAATTGGGCTCGATTTTCCTATTT
CTACAAGGAGCAGCCTTTAGACTTGATTAAGAATTATTATGGAtaactcgagCCCTTCAGAT
TAAAAATAACTGAGGTAAGGGCCTGGGTAGGGGAGGTGGTGTGAGACGCTCCTGTCTC
TCCTCTATCTGCCCATCGGCCCTTTGGGGAGGAGGAATGTGCCCAAGGACTAAAAAAA
GGCCATGGAGCCAGAGGGGCGAGGGCAACAGACCTTTCATGGGCAAACCTTGGGGCC
CTGCTGactgtaGATGAGAGCAGCCACTACGGGTCTAGGCTGCCCATGTAAGGAGGCAAG
GCCTGGGGACACCCGAGATGCCTGGTTATAATTAACCCAGACATGTGGCTGCCCCCCC
CCCCCCAACACCTGCTGCCTGCTAAAAATAACCCTGTCCCTGGTGGccctgcatgcccTTCGA
ACAAGGCTGTGGGGGACTGAGGGCAGGCTGTAACAGGCTTGGGGGCCAGGGCTTATAC
GTGCCTGGGACTCCCAAAGTATTACTGTTCCATGTTCCCGGCGAAGGGCCAGCTGTCCC
CCGCCAGCTAGACTCAGCACTTAGTTTAGGAACCAGTGAGCAAGTCAGCCCTTGGGGC
AGCCCATACAAGGCCATGGGGCTGGGCAAGCTGCACGCCTGGGTCCGGGGTGGGCACG
GTGCCCGGGCAACGAGCTGAAAGCTCATCTACTCTCAGGGGCCCCTCCCTGGGGACAG
CCCCTCCTGGCTAGTCACACCCTGTAGGCTCCTCTATATAACCCAGGGGCACAGGGGCT
GCCCtcattctACCACCACCTCCACAGCACAGACAGACACTCAGGAGCCAGCTAGCCTCGA
GcCACCATGAATTATACAGAGTCCAGCCCATTGAGAGAATCAACTGCCATAGGTTTTAC
ACCTGAGTTAGAAAGTATCATACCTGTGCCTTCCAATAAGACCACTTGTGAAAACTGG
AGAGAGATACATCATCTGGTTTTTCATGTAGCAAATATTTGTTTTGCAGTTGGGTTGGT
TATTCCAACTACTCTTCACCTTCATATGATATTTCTTAGGGGAATGTTAACTCTAGGATG
TACCCTTTATATCGTCTGGGCCACTCTCTACCGATGTGCCTTGGATATAATGATCTGGA
ACTCTGTGTTCTTGGGTGTCAACATTTTGCATCTGTCGTATCTTTTATACAAGAAGAGAC
CGGTAAAGATTGAAAAGGAACTCAGTGGCATGTACCGGCGATTGTTTGAACCACTCCG
TGTGCCTCCAGATTTGTTCAGAAGACTAACTGGACAGTTTTGCATGATCCAAACCTTGA
AAAAGGGCCAAACTTATGCTGCAGAGGATAAAACCTCAGTTGATGACCGTCTGAGTAT
TCTCTTGAAGGGAAAAATGAAGGTCTCCTATCGAGGACATTTTCTGCATAACATTTACC
CCTGTGCCTTTATAGATTCTCCTGAATTTAGATCAACTCAGATGCACAAAGGTGAAAAA
TTCCAGGTCACCATTATTGCAGATGATAACTGCAGATTTTTATGCTGGTCAAGAGAAAG
ATTAACATACTTTCTGGAATCAGAACCTTTCTTGTATGAAATCTTTAGGTATCTTATTGG
AAAAGACATCACAAATAAGCTCTACTCATTGAATGATCCCACCTTAAATGATAAAAAA
GCCAAAAAGCTGGAACATCAGCTCAGCCTCTGCACACAGATCTCCATGTTGGAAATGA
GGAACAGTATAGCCAGCTCCAGTGACAGTGACGACGGCTTGCACCAGTTTCTTCGGGG
TACCTCCAGCATGTCCTCTCTTCATGTGTCATCCCCACACCAGCGAGCCTCTGCCAAGA
TGAAACCGATAGAAGAAGGAGCAGAAGATGATGATGACGTTTTTGAACCGGCATCTCC
AAATACATTGAAAGTCCATCAGCTGCCTggatcctacccatacgatgttccagattacgcttac
ccatacgatgttccagattacgcttacccatacgatgttccagattacgcttaagaattcctag
agctcgctgatcagcctcgactgtgccttctagttgccagccatctgttgtttgcccctccccc
gtgccttccttgaccctggaaggtgccactcccactgtcctttcctaataaaatgaggaaattg
catcgcattgtctgagtaggtgtcattctattctggggggtggggggggcaggacagcaagggg
gaggattgggaagagaatagcaggcatgctggggacgtacgaagaaGCggccgcaggaacccct
agtgatggagttggccactccctctctgcgcgctcgctcgctcactgaggccgggcgaccaaag
gtcgcccgacgcccgggctttgcccgggcggcctcagtgagcgagcgagcgcgcagctgcctgc
aggggcgcctgatgcggtattttctccttacgcatctgtgcggtatttcacaccgcatacgtca
aagcaaccatagtacgcgccctgtagcggcgcattaagcgcggcgggtgtggtggttacgcgca
gcgtgaccgctacacttgccagcgccctagcgcccgctcctttcgctttcttcccttcctttct
cgccacgttcgccggctttccccgtcaagctctaaatcgggggctccctttagggttccgattt
agtgctttacggcacctcgaccccaaaaaacttgatttgggtgatggttcacgtagtgggccat
cgccctgatagacggtttttcgccctttgacgttggagtccacgttctttaatagtggactctt
gttccaaactggaacaacactcaaccctatctcgggctattcttttgatttataagggattttg
ccgatttcggcctattggttaaaaaatgagctgatttaacaaaaatttaacgcgaattttaaca
aaatattaacgtttacaattttatggtgcactctcagtacaatctgctctgatgccgcatagtt
aagccagccccgacacccgccaacacccgctgacgcgccctgacgggcttgtctgctcccggca
tccgcttacagacaagctgtgaccgtctccgggagctgcatgtgtcagaggttttcaccgtcat
caccgaaacgcgcgagacgaaagggcctcgtgatacgcctatttttataggttaatgtcatgat
aataatggtttcttagacgtcaggtggcacttttcggggaaatgtgcgcggaacccctatttgt
ttatttttctaaatacattcaaatatgtatccgctcatgagacaataaccctgataaatgcttc
aataatattgaaaaaggaagagtatgagtattcaacatttccgtgtcgcccttattcccttttt
tgcggcattttgccttcctgtttttgctcacccagaaacgctggtgaaagtaaaagatgctgaa
gatcagttgggtgcacgagtgggttacatcgaactggatctcaacagcggtaagatccttgaga
gttttcgccccgaagaacgttttccaatgatgagcacttttaaagttctgctatgtggcgcggt
attatcccgtattgacgccgggcaagagcaactcggtcgccgcatacactattctcagaatgac
ttggttgagtactcaccagtcacagaaaagcatcttacggatggcatgacagtaagagaattat
gcagtgctgccataaccatgagtgataacactgcggccaacttacttctgacaacgatcggagg
accgaaggagctaaccgcttttttgcacaacatgggggatcatgtaactcgccttgatcgttgg
gaaccggagctgaatgaagccataccaaacgacgagcgtgacaccacgatgcctgtagcaatgg
caacaacgttgcgcaaactattaactggcgaactacttactctagcttcccggcaacaattaat
agactggatggaggcggataaagttgcaggaccacttctgcgctcggcccttccggctggctgg
tttattgctgataaatctggagccggtgagcgtggaagccgcggtatcattgcagcactggggc
cagatggtaagccctcccgtatcgtagttatctacacgacggggagtcaggcaactatggatga
acgaaatagacagatcgctgagataggtgcctcactgattaagcattggtaactgtcagaccaa
gtttactcatatatactttagattgatttaaaacttcatttttaatttaaaaggatctaggtga
agatcctttttgataatctcatgaccaaaatcccttaacgtgagttttcgttccactgagcgtc
agaccccgtagaaaagatcaaaggatcttcttgagatcctttttttctgcgcgtaatctgctgc
ttgcaaacaaaaaaaccaccgctaccagcggtggtttgtttgccggatcaagagctaccaactc
tttttccgaaggtaactggcttcagcagagcgcagataccaaatactgtccttctagtgtagcc
gtagttaggccaccacttcaagaactctgtagcaccgcctacatacctcgctctgctaatcctg
ttaccagtggctgctgccagtggcgataagtcgtgtcttaccgggttggactcaagacgatagt
taccggataaggcgcagcggtcgggctgaacggggggttcgtgcacacagcccagcttggagcg
aacgacctacaccgaactgagatacctacagcgtgagctatgagaaagcgccacgcttcccgaa
gggagaaaggcggacaggtatccggtaagcggcagggtcggaacaggagagcgcacgagggagc
ttccagggggaaacgcctggtatctttatagtcctgtcgggtttcgccacctctgacttgagcg
tcgatttttgtgatgctcgtcaggggggggagcctatggaaaaacgccagcaacgcggcctttt
tacggttcctggccttttgctggccttttgctcacatgt
  9. SEQ ID NO: 9 - Bves KO Forward Primer
ACTTGCTTTAAAAAACCTCCCACA
10. SEQ ID NO: 10 - Bves KO Reverse Primer
AGTCACTAGCAAGAGATCTGCACCC
11. SEQ ID NO: 11 - Bves WT Forward Primer
AAGTGCTGGGATTAAAGGTGTGTGC
12. SEQ ID NO: 12 - Bves WT Reverse Primer
AAGGACACATCACAGCTTCAGG
13. SEQ ID NO: 13 - Astrogin1 Forward Primer
TAGTAAGGCTGTTGGAGCTGATAG
14. SEQ ID NO: 14 - Astrogin1 Reverse Primer
CTGCACCAGTGTGCATAAGG
15. SEQ ID NO: 15 - MuRF1 Forward Primer
CATCTTCCAGGCTGCGAATC
16. SEQ ID NO: 16 - MuRF1 Reverse Primer
ACTGGAGCACTCCTGCTTGT
17. SEQ ID NO: 17 - MUSA1 Forward Primer
CTTCAGTCTCGTGGAATGGTAATCTT
18. SEQ ID NO: 18 - MUSA1 Reverse Primer
TGCAGTACTGAATCGCCATAC
19. SEQ ID NO: 19 - Bnip3 Forward Primer
TCCTGGGTAGAACTGCACTTC
20. SEQ ID NO: 20 - Bnip3 Reverse Primer
GCTGGGCATCCAACAGTATTT
21. SEQ ID NO: 21 - p62 Forward Primer
AGGATGGGGACTTGGTTGC
22. SEQ ID NO: 22 - p62 Reverse Primer
TCACAGATCACATTGGGGTGC
23. SEQ ID NO: 23 - Beclin1 Forward Primer
TGGAAGGGTCTAAGACGT
24. SEQ ID NO: 24 - Beclin1 Reverse Primer
GGCTGTGGTAAGTAATGGA
25. SEQ ID NO: 25 - Cts1 Forward Primer
GTGGACTGTTCTCACGCTCAAG
26. SEQ ID NO: 26 - Cts Reverse Primer
TCCGTCCTTCGCTTCATAGG
27. SEQ ID NO: 27 - GAPDH Forward Primer
AGGTCGGTGTGAACGGATTTG
28. SEQ ID NO: 28 - GAPDH Reverse Primer
TGTAGACCATGTAGTTGAGGTCA
29. SEQ ID NO: 29 - BVES Forward Primer
CCGTGCCTTCTAATGAGACCA
30. SEQ ID NO: 30 - BVES Reverse Primer
AGACCACATAAAGGGTACATCCT
31. SEQ ID NO: 31 - POPDC2 Forward Primer
AGGAGCTGGAAACCGGATGTA
32. SEQ ID NO: 32 - POPDC2 Reverse Primer
GACGATGTCTAGTCCACAAGC
33. SEQ ID NO: 33 - POPDC3 Forward Primer
TGACTGAACACCCACTCTGC
34. SEQ ID NO: 34 - POPDC3 Reverse Primer
ACTGCCACCCATAAAACCTACT
35. SEQ ID NO: 35 - ATG7 Forward Primer
TCTGGGAAGCCATAAAGTCAGG
36. SEQ ID NO: 36 - ATG7 Reverse Primer
GCGAAGGTCAGGAGCAGAA
37. SEQ ID NO: 37 - PARK2 Forward Primer
TCTTCCAGTGTAACCACCGTC
38. SEQ ID NO: 38 - PARK2 Reverse Primer
GGCAGGGAGTAGCCAAGTT
39. SEQ ID NO: 39 - hADCY3-E4gRNA Forward Primer
ACCGCGTACCTGACGTTCTCGTGA
40. SEQ ID NO: 40 - hADCY3-E4gRNA Reverse Primer
AAACTCACGAGAACGTCAGGTACG
41. SEQ ID NO: 41 - hADCY3 Forward Primer
ACCTCATGCTTTCCATCCTG
42. SEQ ID NO: 42 - hADCY3 Reverse Primer
CCGCTCCAGGTGATATCTGT
43. SEQ ID NO: 43 - hADCY6-Q205gRNA Forward Primer
ACCGCCGCCAGGACTCCATGTGGG
44. SEQ ID NO: 44 - hADCY6-Q205gRNA Reverse Primer
AAACCCCACATGGAGTCCTGGCGG
45. SEQ ID NO: 45 - hADCY6 Forward Primer
AGGATACCGAGGTGACAACG
46. SEQ ID NO: 46 - hADCY6 Reverse Primer
GCCAAGATCAAATGCAAGGT
```

The invention claimed is:

1. A method of treating muscular dystrophy in a subject in need thereof, comprising administering to the subject a therapeutically effective amount of an expression vector comprising a nucleic acid encoding a blood vessel epicardial substance (BVES) protein.

2. The method of claim 1, wherein the nucleic acid is an RNA or a DNA.

3. The method of claim 1, wherein the nucleic acid encoding the BVES protein comprises a sequence at least 70% identical to SEQ ID NO: 1, or a fragment thereof.

4. (canceled)

5. (canceled)

6. The method of claim 1, wherein the nucleic acid encoding the BVES protein comprises SEQ ID NO:1, or a fragment thereof.

7. The method of claim 1, wherein the nucleic acid encoding the BVES protein is operably linked to a muscle-specific promoter.

8. The method of claim 7, wherein the muscle-specific promoter is MHCK7.

9. The method of claim 8, wherein the MHCK7 promoter comprises a sequence at least 70% identical to SEQ ID NO: 3, or a fragment thereof.

10. (canceled)

11. (canceled)

12. The method of claim 8, wherein the MHCK7 promoter comprises SEQ ID NO: 3, or a fragment thereof.

13. The method of claim 1, wherein the nucleic acid encoding the BVES protein is operatively linked to an inverted terminal repeat (ITR) sequence.

14. The method of claim 13, wherein the ITR sequence is at least 70% identical to SEQ ID NO: 4, or a fragment thereof.

15. (canceled)

16. (canceled)

17. The method of claim 13, wherein the ITR sequence comprises SEQ ID NO: 4, or a fragment thereof.

18. The method of claim 1, wherein the expression vector comprises a sequence at least 70% identical SEQ ID NO: 8, or a fragment thereof.

19. (canceled)

20. (canceled)

21. The method of claim 1, wherein the expression vector comprises SEQ ID NO: 8, or a fragment thereof.

22. The method of claim 1, wherein the expression vector is a viral vector.

23. The method of claim 22, wherein the viral vector is an adeno-associated virus (AAV) vector.

24. The method of claim 23, wherein the AAV vector is AAV9.

25. The method of claim 1, wherein the subject has limb-girdle muscular dystrophy type R25 (LGMDR25).

26. The method of claim 1, wherein the subject has a mutated BVES gene.

27. The method of claim 1, further comprising administering to the subject a therapeutically effective amount of a proteasome inhibitor.

28. The method of claim 27, wherein the proteasome inhibitor is bortezomib.
